(19) United States
(12) Patent Application Publication
(10) Pub. No.: US 2023/0111910 A1
(43) Pub. Date: Apr. 13, 2023

(54) PD-1/PD-L1 INHIBITOR, PREPARATION METHOD THEREFOR, AND USE THEREOF

(71) Applicants: SHANGHAI SYNERGY PHARMACEUTICAL SCIENCES CO., LTD., Shanghai (CN); ZHEJIANG HUAHAI PHARMACEUTICAL CO., LTD., Zhejiang (CN)

(72) Inventors: Xin XU, Shanghai (CN); Jia CHEN, Shanghai (CN); Liming ZHANG, Shanghai (CN); Xuqin YANG, Shanghai (CN); Maozhi YANG, Shanghai (CN); Xiaojuan ZHANG, Shanghai (CN); Yanxiao XU, Shanghai (CN); Xiaobo ZHOU, Shanghai (CN); Hao YU, Shanghai (CN); Yuyun ZHANG, Shanghai (CN); Ying WANG, Shanghai (CN); Xiaoer XIA, Shanghai (CN)

(21) Appl. No.: 17/794,390

(22) PCT Filed: Jan. 21, 2021
(86) PCT No.: PCT/CN2021/073025

§ 371 Date: Jul. 21, 2022

(30) Foreign Application Priority Data
Jan. 21, 2020 (CN) 202010070965.3

(51) Int. Cl. C07D 519/00; C07D 401/12; C07D 417/12; C07D 513/04; C07D 471/04; A61P 35/00

(52) U.S. Cl. CPC C07D 519/00; C07D 401/12; C07D 417/12; C07D 513/04; C07D 471/04; A61P 35/00

(57) ABSTRACT

Provided are a compound as a PD-L1 inhibitor, a preparation method therefor, and use thereof. Further provided is use of the compound or a pharmaceutical composition thereof in the preparation of a medicine, wherein the medicine is used for preventing and treating a PD-L1-related disease.

The present application claims the priority of the Chinese patent application No. 202010070965.3, titled “PD-1/PD-L1 INHIBITOR, PREPARATION METHOD THEREFOR, AND USE THEREOF”, filed before CNIPA on Jan. 21, 2020, which is hereby incorporated by reference in its entirety.

### FIELD OF THE INVENTION

The invention belongs to the field of medicine, and relates to a small molecule compound with PD-1/PD-L1 inhibitory activity, a preparation method thereof, a pharmaceutical composition containing the compound, and use thereof in medicine.

### BACKGROUND OF THE INVENTION

Cancer is one of the diseases that seriously threaten human health. The prevention and treatment of cancer has become a very important public health issue. Along with surgery, radiotherapy and chemotherapy, tumor immunotherapy is one of the four major tumor treatments, and is currently considered as a tumor treatment method with great potential. Immune checkpoint inhibitor, a hot research direction in the field, has been made significant clinical research progress, which provides a new weapon for fighting against cancer. PD-1/PD-L1 is one of the most popular immune checkpoints nowadays.

PD-1 (CD279), programmed death receptor 1, is an important immunosuppressive molecule. PD-1 consists of 288 amino acids and belongs to immunoglobulin superfamily CD28. It is expressed on the surface of various immune cells such as activated T cells, B cells, natural killer (NK) cells, and antigen-presenting cells, and is up-regulated on the surface of activated T cells in tumor patients. The corresponding receptor PD-L1 (B7-H1, CD274) is mainly expressed in a variety of tumor cells, T cells, APCs and non-hematopoietic cells. The interaction between PD-1 and PD-L1 inhibits T cell activation, which is essential for maintaining immunologic tolerance in normal organisms. The tumor microenvironment in organism will induce infiltrating T cells to highly express PD-1 molecules, and tumor cells will highly express PD-L1 and PD-L2, resulting in the continuous activation of the PD-1/PD-L1 signaling pathway in the tumor microenvironment. The functions of T cells are inhibited thereby, resulting in immune escape of tumor cells.

Several antibodies blocking this signaling pathway are currently approved. Multiple studies have demonstrated that these antibodies are effective in a variety of tumors, such as melanoma, non-small cell lung cancer, renal cell carcinoma, gastric cancer, bladder cancer, Hodgkin lymphoma, and the like.

In conclusion, blockers of PD-1/PD-L1/PD-L2 signaling pathway have achieved encouraging therapeutic effects in clinical tumor immunotherapy. Although monoclonal antibodies against certain cancers have been successfully developed, the research and development costs are high, and the monoclonal antibodies are unstable and have potential immunogenic side effects. In contrast, small molecule inhibitors targeting PD-1/PD-L1 have attracted more attention.

The present invention provides a compound of general formula (I) which inhibits PD-1/PD-L1 interaction, and a stereoisomer and a pharmaceutically acceptable salt thereof, a preparation method and pharmaceutical composition thereof, and use thereof in a medicament for preventing and treating diseases related to PD-1/PD-L1 signaling pathway.

### SUMMARY OF THE INVENTION

The present invention aims to provide a compound represented by general formula (I), and a tautomer, enantiomer, diastereomer, racemate and pharmaceutically acceptable salt thereof.

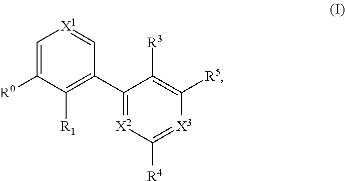

wherein

X¹ is C—R², X² is C, and X³ is C;

X¹ is N, X² is C, and X³ is C;

X¹ is C—R², X² is C, and X³ is N;

X¹ is C—R², X² is N, and X³ is C; or

X¹ is C—R², X² is N, and X³ is N; and

R⁰ is NR^{a}R⁶ or —NR^{a}C(O)R⁶;

R¹, R³ and R⁴ are each independently hydrogen, halogen or C₁₋₆ alkyl;

R² is hydrogen or C₁₋₆ alkyl;

R⁵ is C₅₋₁₂ fused heterobicyclic group, —NR^{b}R⁷ or —NR^{b}C(O)R⁷, wherein optionally one or more hydrogen atom of the C₅₋₁₂ fused heterobicyclic group is independently substituted by R^{d};

R⁶ is C₃₋₇ heterocycloalkyl, C₃₋₉ heteroaryl, C₆₋₁₂ fused bicyclic group or C₅₋₁₂ fused heterobicyclic group, wherein optionally one or more hydrogen atom on R⁶ is independently substituted by R^{e};

R⁷ is C₃₋₇ heterocycloalkyl, C₃₋₉ heteroaryl, C₆₋₁₂ fused bicyclic group, C₅₋₁₂ fused heterobicyclic group, wherein one or more hydrogen atom on R⁷ can be independently substituted by R^{f};

R^{a} and R^{b} are each independently hydrogen, —CH₃ or —CH(CH₃)₂;

R^{d} is independently halogen, cyano, C₁₋₆ alkyl, C₃₋₇ heterocycloalkyl or -methylene-C₃₋₇ heterocycloalkyl, wherein said C₁₋₆ alkyl, C₃₋₇ heterocycloalkyl, or -methylene-C₃₋₇ heterocycloalkyl can be substituted by —CH₃, —OH, —COOH, or —COOCH₃;

R^{e} and R^{f} are each independently hydrogen, C₁₋₆ alkyl, —(CH₂)ₙ—OH, —(CH₂)ₙ—NH₂, —(CH₂)ₙ—NH—(CH₂)ₙ—CH₃, —O—CH₃, C₃₋₆ cycloalkyl, C₃₋₇ heterocycloalkyl, -methylene-C₃₋₇ heterocycloalkyl or C₆₋₁₀ aryl, wherein said C₁₋₆ alkyl, —(CH₂)ₙ—OH, —(CH₂)ₙ—NH₂, —(CH₂)ₙ—NH—(CH₂)ₙ—CH₃, C₃₋₆ cycloalkyl, C₃₋₇ heterocycloalkyl, -methylene-C₃₋₇ heterocycloalkyl, or C₆₋₁₀ aryl can be substituted by IV;

R^{j} is halogen, hydroxyl, —NH₂, C₁₋₆ alkyl, —COOH, —COOCH₃, —(CH₂)ₙ—OH, —(CH₂)ₙ—NH—CH₃, —(CH₂)ₙ—O—CH₃ or —(CH₂)ₙ—NH—C(O)—CH₃;

n is 0, 1 or 2.

Preferably, in the compound represented by general formula (I),

R⁰ is —NR^{a}R⁶ or —NR^{a}—C(O)—R⁶,

R¹, R³ and R⁴ are each independently —H, —F, —Cl or —CH₃,

R⁵ is —NR^{b}R⁷, —NR^{b}—C(O)—R⁷,

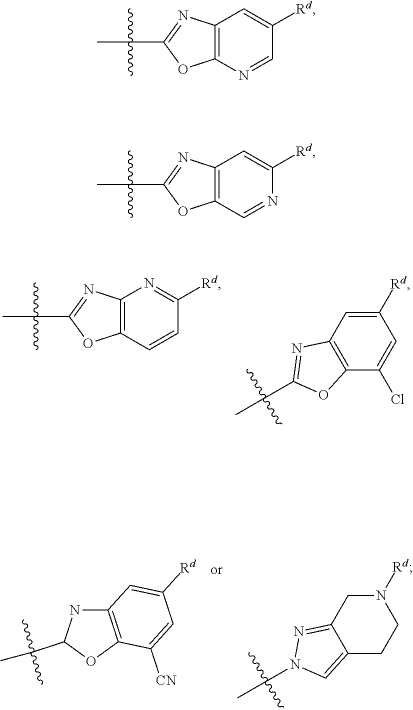

R⁶ is

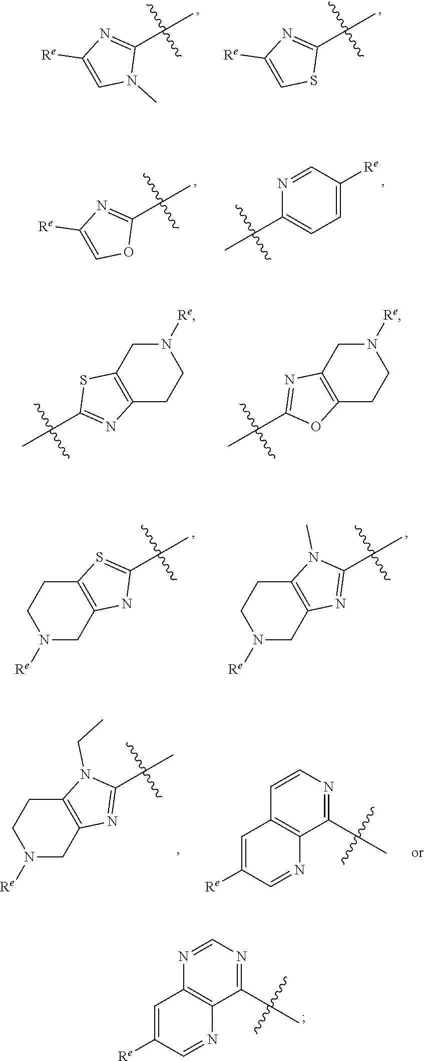

R⁷ is

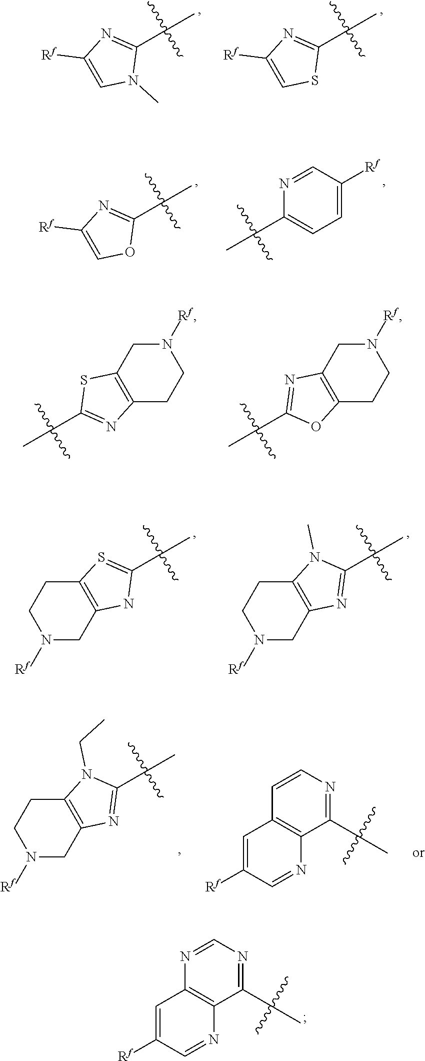

R^{a} and R^{b} are each independently —H, —CH₃ or —CH(CH₃)₂;

R^{d} is independently —CH(CH₃)₂,

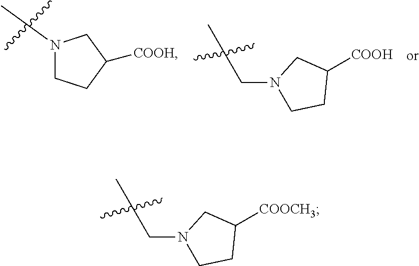 [SMILES: *N1CCC(C(=O)O)C1 ; *CN1CCC(C(=O)O)C1 ; *CN1CCC(C(=O)OC)C1]

R^{e} and R^{f} are each independently —H, —CH₃, —CH₂CH₃, —CH(CH₃)₂, —CH₂CH(CH₃)OH, —CH₂CH(OH)CH₃,

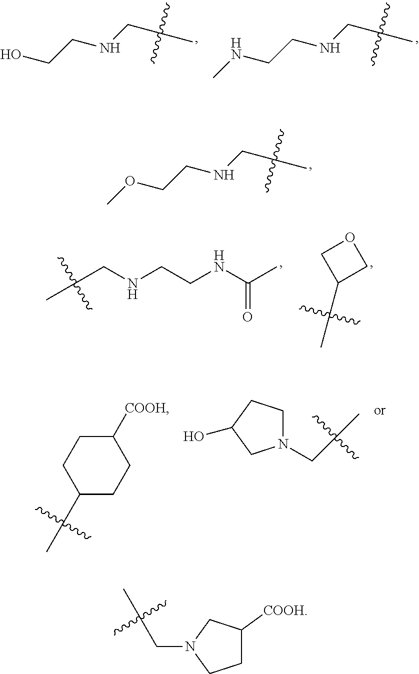

Further preferably, in the compound represented by general formula (I),

R⁰ is —NH—C(O)—R⁶, —NH—R⁶, —N(CH₃)—C(O)—R⁶ or —N(CH(CH₃)₂)—C(O)—R⁶;

R⁵ is —NH—C(O)—R⁷, —NH—R⁷, —N(CH₃)—C(O)—R⁷, —N(CH(CH₃)₂)—C(O)—R⁷,

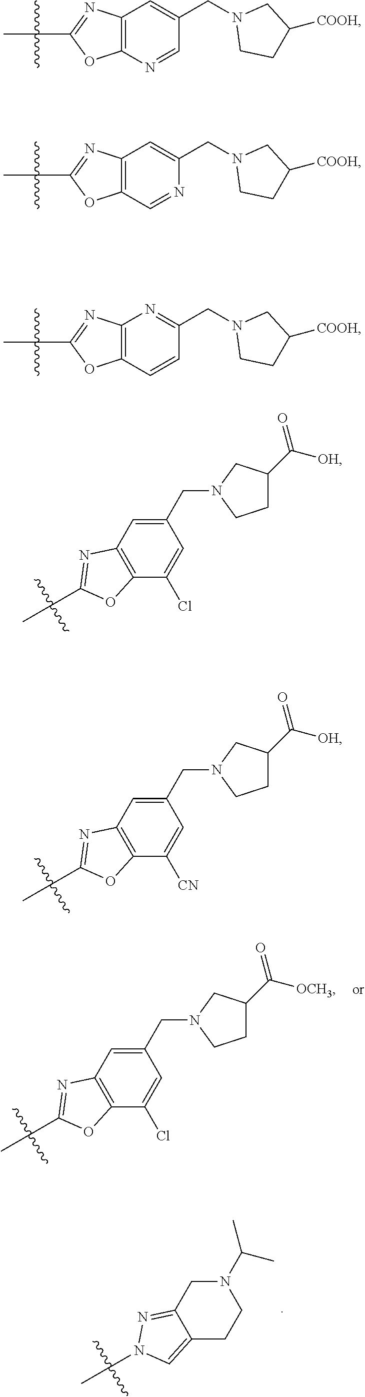

Preferably, in the compound represented by general formula (I),

X¹ is C—R², X² is C, and X³ is C;

R⁰ is —NH—C(O)—R⁶ or —NH—R⁶;

R¹ is —Cl or —CH₃;

R² is —H;

R³ is —Cl or —CH₃;

R⁴ is —H;

R⁵ is —NH—C(O)—R⁷,

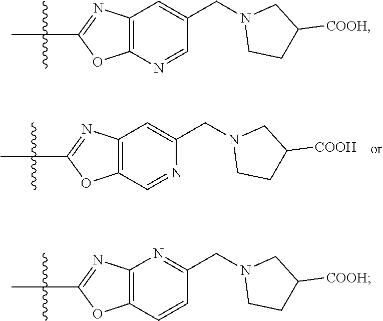 [SMILES: *c1nc2cc(CN3CCC(C(=O)O)C3)cnc2o1 ; *c1nc2cc(CN3CCC(C(=O)O)C3)ncc2o1 ; *c1nc2nc(CN3CCC(C(=O)O)C3)ccc2o1]

R⁶ is

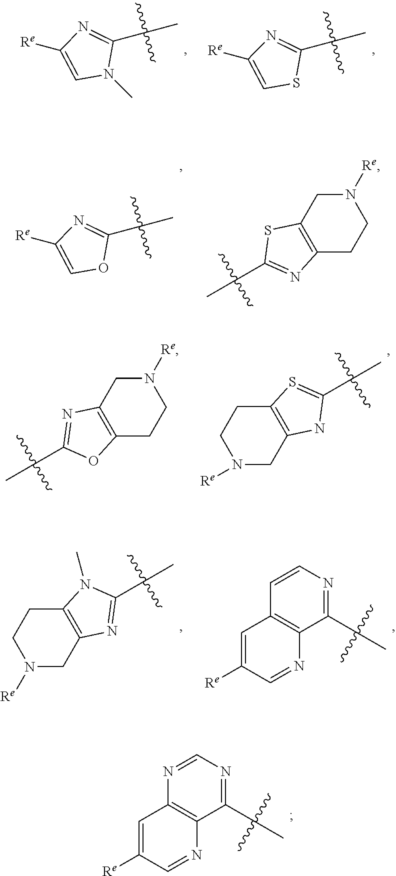

R⁷ is

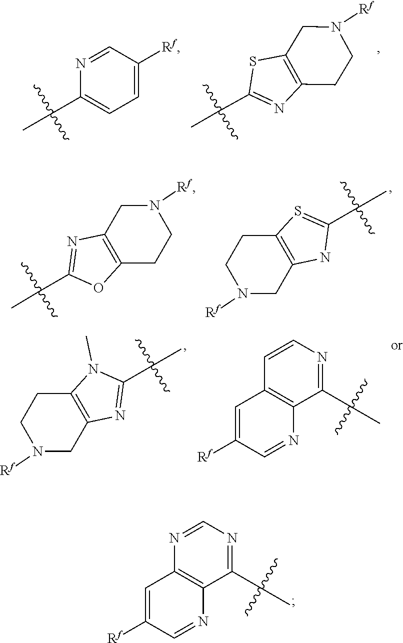

R^{e} is —H, —CH₃, —CH₂CH(CH₃)OH,

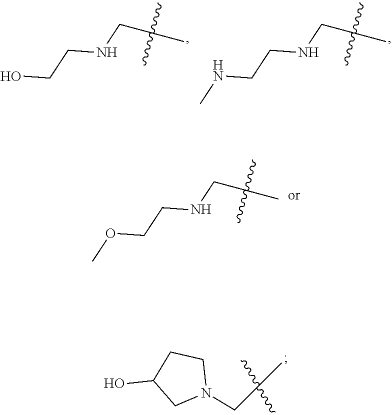

R^{f} is —CH₃,

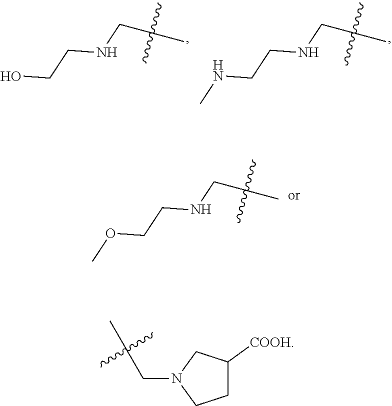

Preferably, in the compound represented by general formula (I),

X¹ is N, X² is C, X³ is C;

R⁰ is —NH—C(O)—R⁶ or —NH—R⁶;

R¹ is —CH₃;

R³ is —Cl or —CH₃;

R⁴ is —H or —CH₃;

R⁵ is —NH—C(O)—R⁷,

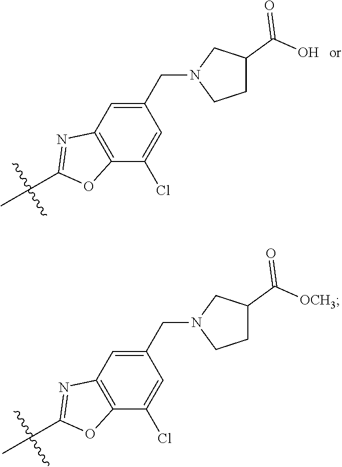 [SMILES: *c1nc2cc(CN3CCC(C(=O)O)C3)cc(Cl)c2o1 ; *c1nc2cc(CN3CCC(C(=O)OC)C3)cc(Cl)c2o1]

R⁶ is

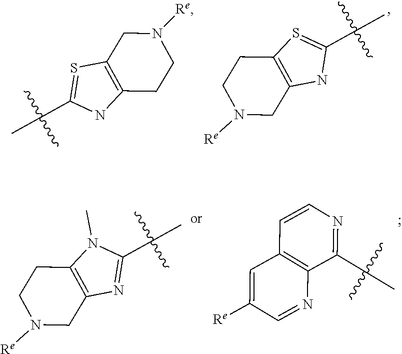

R⁷ is

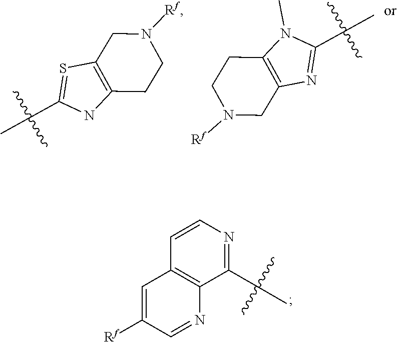

R^{e} is —H, —CH₃, —CH₂CH₃, —CH(CH₃)₂, —CH₂CH(CH₃)OH or

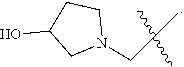 [SMILES: *CN1CCC(O)C1]

R^{f} is —CH₃, —CH₂CH₃, —CH(CH₃)₂, —CH₂CH(CH₃)OH, —CH₂CH(OH)CH₃,

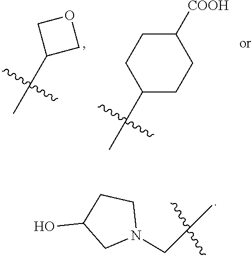 [SMILES: *C1COC1 ; *C1CCC(C(=O)O)CC1 ; *CN1CCC(O)C1]

Preferably, in the compound represented by general formula (I),

X¹ is C—R², X² is C, X³ is N;

R⁰ is —NH—C(O)—R⁶;

R¹ is —Cl or —CH₃;

R² is —H;

R³ is —H, —Cl or —CH₃;

R⁴ is —H;

R⁵ is —NH—C(O)—R⁷;

R⁶ is

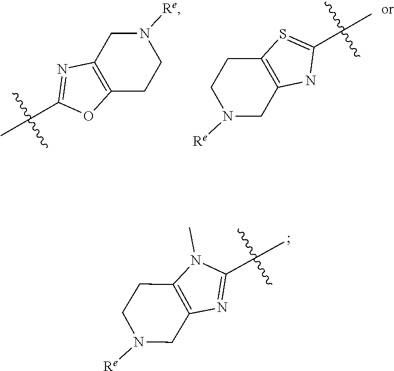

R⁷ is

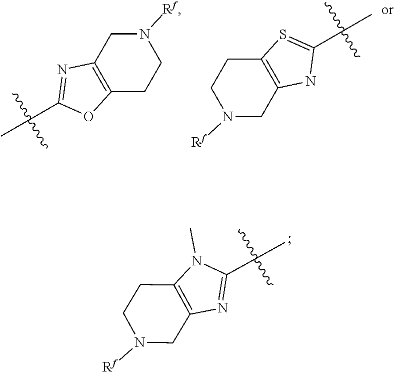

R^{e} is —CH₃ or —CH₂CH(CH₃)OH;

R^{f} is —CH₃ or —CH₂CH(CH₃)OH.

Preferably, in the compound represented by general formula (I),

X¹ is C—R², X² is N, X³ is C;

R⁰ is —NH—C(O)—R⁶, —N(CH₃)—C(O)—R⁶ or —N(CH(CH₃)₂)—C(O)—R⁶;

R¹ is —Cl or —CH₃;

R² is —H, —F or —Cl;

R³ is —Cl or —CH₃;

R⁴ is —H;

R⁵ is —NH—C(O)—R⁷, —N(CH₃)—C(O)—R⁷ or —N(CH(CH₃)₂)—C(O)—R⁷;

R⁶ is

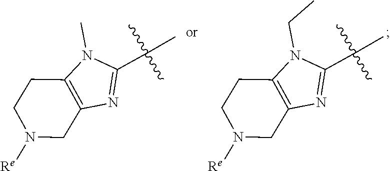

R⁷ is

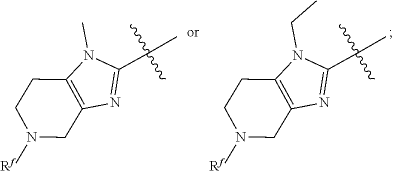

R^{e} is —CH₃ or —CH₂CH(CH₃)OH;

R^{f} is —CH₃.

Preferably, in the compound represented by general formula (I),

X¹ is C—R², X² is N, X³ is N;

R⁰ is —NH—C(O)—R⁶;

R¹ is —Cl or —CH₃;

R² is —H;

R³ is —Cl or —CH₃;

R⁴ is —H;

R⁵ is —NH—C(O)—R⁷;

R⁶ is

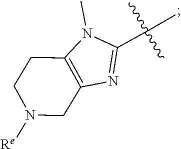

R⁷ is

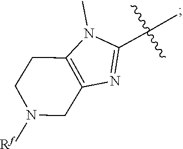

R^{e} is —CH₃ or —CH₂CH(CH₃)OH;

R^{f} is —CH₃ or —CH₂CH(CH₃)OH.

Further preferably, the compound represented by general formula (I) is selected from the group consisting of:
N-(2-chloro-3′-(4-(((2-hydroxyethyl)amino)methyl)-1-methyl-1H-imidazole-2-carboxamido)-2′-methyl-[1,1′-biphenyl]-3-yl)-5-(((2-hydroxyethyl)amino)methyl)picolinamide;
N-(2′-chloro-3′-(5-((((2-hydroxyethyl)amino)methyl)picolinamido)-2-methyl-[1,1′-biphenyl]-3-yl)-4-(((2-hydroxyethyl)amino)methyl)thiazole-2-carboxamide;
(S)—N-(5-(3-(1,5-dimethyl-4,5,6,7-tetrahydro-1H-imidazo[4,5-c]pyridine-2-carboxamido)-2-methylphenyl)-4-methylpyridin-3-yl)-5-(2-hydroxypropyl)-1-methyl-4,5,6,7-tetrahydro-1H-imidazo[4,5-c]pyridine-2-carboxamide;
(R)-1-(((2-(2,2′-dimethyl-3′-(5-methyl-4,5,6,7-tetrahydrothiazolo[5,4-c]pyridine-2-carboxamido))-[1,1′-biphenyl]-3-yl)oxazolo[5,4-b]pyridin-6-yl)methyl)pyrrolidine-3-carboxylic acid;
N-(5-(3-(1,5-dimethyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide;
N-(5-(3-(5-(2-hydroxypropyl)-1-methyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide;
(R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[5,4-b]pyridin-6-yl)methyl)pyrrolidine-3-carboxylic acid;
(R)-1-((7-chloro-2-(2-methyl-3-(4-methyl-5-(5-methyl-4,5,6,7-tetrahydrothiazolo[5,4-c]pyridine-2-carboxamido)pyridin-3-yl)phenyl)benzo[d]oxazol-5-yl)methyl)pyrrolidine-3-carboxylic acid;
(R)-1-(((7-chloro-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid;
(R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridin-6-yl)methyl)pyrrolidine-3-carboxylic acid;
(R)-1-(((7-chloro-2-(5-(3-((3-((((R)-3-hydroxypyrrolidin-1-yl)methyl)-1,7-naphthyridin-8-yl)amino)-2-methylphenyl)-4-methylpyridin-3-yl)benzo[d]oxazol-5-yl)methyl)pyrrolidine-3-carboxylic acid trifluoroacetate;
(R)-1-(((7-chloro-2-(5-(3-(5-((S)-2-hydroxypropyl))-1-methyl-4,5,6,7-tetrahydro-1H-imidazo[4,5-c]pyridine-2-carboxamide)-2-methylphenyl)-4-methylpyridin-3-yl)benzo[d]oxazol-5-yl)methyl)pyrrolidine-3-carboxylic acid;
(R)-1-(((7-chloro-2-(5-(3-(1,5-dimethyl-4,5,6,7-tetrahydro-1H-imidazo[4,5-c]pyridine-2-carboxamido)-2-methylphenyl)-4-methylpyridin-3-yl)benzo[d]oxazol-5-yl)methyl)pyrrolidine-3-carboxylic acid;
(R)-1-((2-(3′-(5-((S)-2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridine-6-yl)methyl)pyrrolidine-3-carboxylic acid;
N-(3-chloro-2-(3-(5-(2-hydroxypropyl)-1-methyl-4,5,6,7-tetrahydro-1H-imidazo[4,5-c]pyridine-2-carboxamido))-2-methylphenyl)pyridin-4-yl)-1,5-dimethyl-4,5,6,7-tetrahydro-1H-imidazo[4,5-c]pyridine-2-carboxamide;
N-(3-(3-chloro-2-(1,5-dimethyl-4,5,6,7-tetrahydro-1H-imidazo[4,5-c]pyridine-2-carboxamido)pyridin-4-yl)-2-methylphenyl)-5-(2-hydroxypropyl)-1-methyl-4,5,6,7-tetrahydro-1H-imidazo[4,5-c]pyridine-2-carboxamide;
(R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[4,5-b]pyridin-5-yl)methyl)pyrrolidine-3-carboxylic acid trifluoroacetate;
(R)-1-((2-(2,2′-dimethyl-3′-(4,5,6,7-tetrahydrothiazolo[5,4-c]pyridine-2-carboxamido)-[1,1′-biphenyl]-3-yl)oxazolo[5,4-b]pyridin-6-yl)methyl)pyrrolidine-3-carboxylic acid trifluoroacetate;
(R)-1-(((7-chloro-2-(4-methyl-5-(2-methyl-3-(1-methyl-4,5,6,7-tetrahydro-1H-imidazole)[4,5-c]pyridine-2-carboxamido)phenyl)pyridin-3-yl)benzo[d]oxazol-5-yl)methyl)pyrrolidine-3-carboxylic acid;
methyl (R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylate;
(R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylic acid;
(R)-1-((2-(3′-(5-((S)-2-hydroxypropyl)-4,5,6,7-tetrahydrothiazolo[5,4-c]pyridine-2-carboxamido)-2,2′-dimethyl-[1,1′-biphenyl]-3-yl)oxazolo[5,4-b]pyridin-6-yl)methyl)pyrrolidine-3-carboxylic acid;
(S)—N-(5-(2-chloro-3-(1-methyl-5-(oxetan-3-yl)-4,5,6,7-tetrahydro-1H-imidazo[4,5-c]pyridine-2-carboxamido)phenyl)-4-methylpyridin-3-yl)-5-(2-hydroxypropyl)-1-methyl-4,5,6,7-tetrahydro-1H-imidazo[4,5-c]pyridine-2-carboxamide;
(S)—N-(2-chloro-3-(5-(5-(2-hydroxypropyl)-1-methyl-4,5,6,7-tetrahydro-1H-imidazo[4,5-c]pyridine-2-carboxamido)-4-methylpyridin-3-yl)phenyl)-5-isopropyl-1-methyl-4,5,6,7-tetrahydro-1H-imidazo[4,5-c]pyridine-2-carboxamide;
(R)-1-(((7-cyano-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid;
N-(5-(2-chloro-3-(5-((S)-2-hydroxypropyl)-1-methyl-4,5,6,7-tetrahydro-1H-imidazo[4,5-c]pyridine-2-carboxamido)phenyl)-4-methylpyridin-3-yl)-5-((S)-2-hydroxypropyl)-1-methyl-4,5,6,7-tetrahydro-1H-imidazo[4,5-c]pyridine-2-carboxamide;
N-(2-chloro-3-(5-(5-ethyl-1-methyl-4,5,6,7-tetrahydro-1H-imidazo[4,5-c]pyridine-2-carboxamido)-4-methylpyridin-3-yl)phenyl)-5-ethyl-1-methyl-4,5,6,7-tetrahydro-1H-imidazo[4,5-c]pyridine-2-carboxamide;
(R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid;
(R)-1-((2-(3′-(5-(2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid;
(R)-1-(((7-chloro-2-(3-(5-(5-((S)-2-hydroxypropyl))-1-methyl-4,5,6,7-tetrahydro-1H-imidazo[4,5-c]pyridine-2-carboxamido)-4-methylpyridin-3-yl)-2-methylphenyl)benzo[d]oxazole-5-yl)methyl)pyrrolidine-3-carboxylic acid.

The compound represented by general formula I can be prepared by the following schemes.

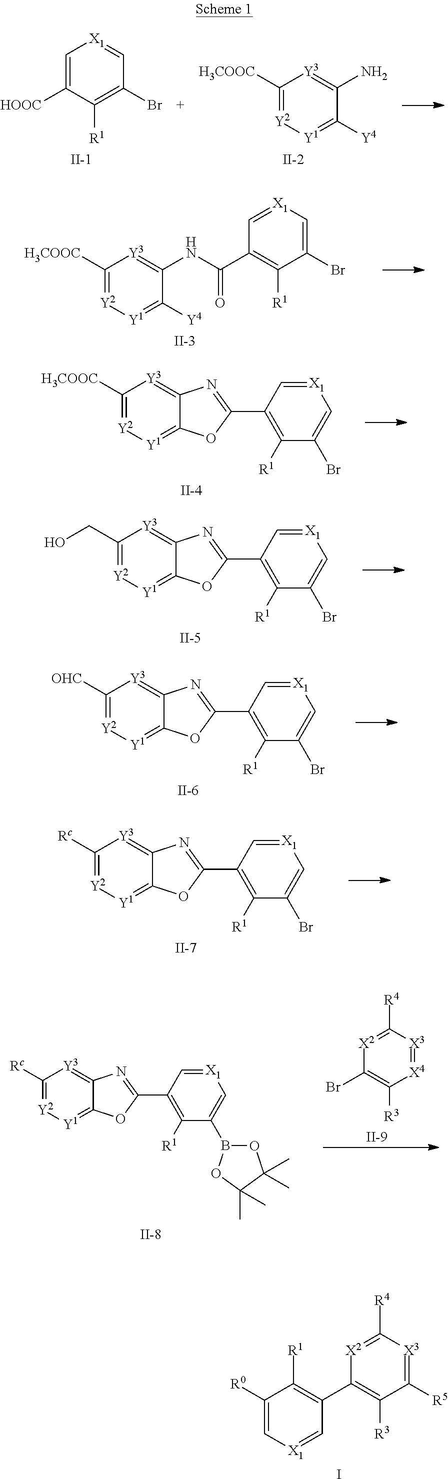

Y₁, Y₂ and Y₃ are N or C, wherein at least two of Y₁, Y₂ and Y₃ are C atoms, or wherein Y₁ is CR, in which R is halogen or —CN, preferably R is-Cl or —CN;

Y₄ is fluoro, chloro, bromo or iodo.

Compound (II-1) is reacted with a corresponding arylamine (II-2) in the presence of a condensing agent under an alkaline condition to obtain a compound (II-3). Compound (II-4) is obtained from compound (II-3) through a ring-closing reaction under the catalysis of a copper salt. The copper salt used is preferably cuprous iodide, cuprous bromide, cuprous chloride and the like. Compound (II-4) is subjected to a three-step reaction including reduction, oxidation and reductive amination to obtain compound (II-7). Compound (II-7) is reacted with diboron pinacol ester in the presence of a catalyst under heating and alkaline conditions to obtain compound (II-8). Compound (II-8) is reacted with compound (II-9) in the presence of a catalyst under heating and alkaline conditions to obtain compound (I).

compound (II-4) can also be synthesized by the following process:

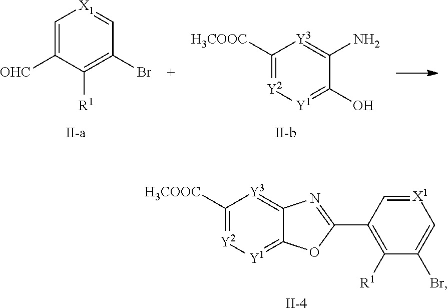

wherein compound (II-4) is obtained through a ring-closing reaction by directly heating compounds (II-a) and (II-b).

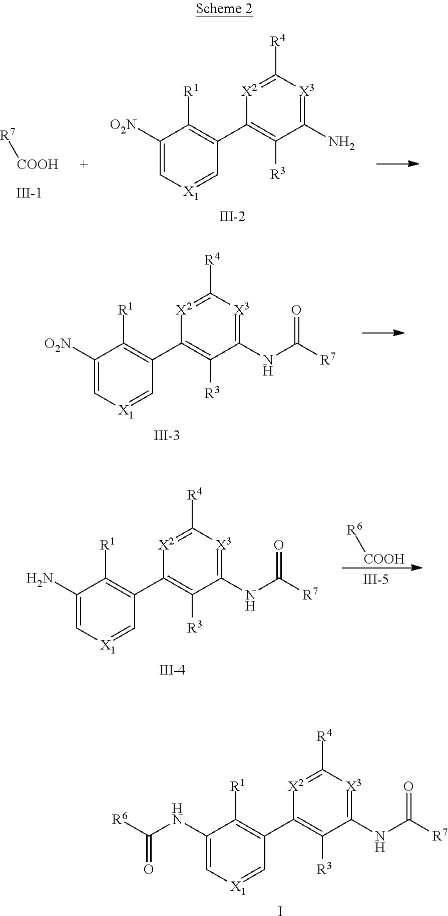

Compound (III-1) is reacted with a corresponding arylamine (III-2) in the presence of a condensing agent under an alkaline condition to obtain compound (III-3). Compound (III-3) is reduced by a reducing agent, and then reacts with a corresponding acid (III-5) under an alkaline condition in the presence of a condensing agent to obtain compound (I).

Unless otherwise defined, all technical and scientific terms used herein have the same meaning as commonly understood by one of ordinary skill in the art.

Unless otherwise stated, conventional methods in the technical field such as mass spectrometry, NMR, IR and UV/VIS spectroscopy and pharmacological methods are used. Unless specific definitions are provided, the terms used herein in the specification related to analytical chemistry, synthetic organic chemistry, and pharmaceutical and medicinal chemistry are known in the art. Standard techniques can be used in chemical synthesis, chemical analysis, drug preparation, formulation and delivery, and treatment of patients. For example, the reaction and purification can be carried out using the manufacturer's instructions for use of the kit, or in a manner well known in the art or as described in the present invention. Generally, the techniques and methods described above can be carried out according to conventional methods well known in the art from the specification in the various general and more specific references cited and discussed in this specification.

When a substituent is described by conventional chemical formulae written from left to right, the substituent also include the chemically equivalent substituent obtained when the structural formula is written from right to left. For example, —CH₂O— is equivalent to —OCH₂—.

The section headings used herein are solely for the purpose of organizing the article and should not be interpreted as limiting the subject matter described. All or part of references cited in this application, including but not limited to patents, patent applications, articles, books, manuals, and papers, are hereby incorporated by reference in their entirety.

In the present invention, “optional” or “optionally” means that the subsequently described event or circumstance may or may not occur, and means that this description includes instances where the event or circumstance occurs or not. For example, “optionally substituted aryl” means that the aryl group is substituted or unsubstituted, and this description includes both the substituted aryl group and unsubstituted aryl group.

When used in reference to a specifically recited value, the term “about” means that the value may vary by no more than 1% from the recited value. For example, as used herein, the expression “about 100” includes all values between 99 and 101 (for example, 99.1, 99.2, 99.3, 99.4, etc.).

The terms “containing” or “including (comprising)” can be open, semi-closed and closed. In other words, the terms also include “consisting essentially of” or “consisting of”.

The term “alkyl” as used herein includes saturated aliphatic groups, including linear alkyl groups (e.g. methyl, ethyl, propyl, butyl, pentyl, hexyl, heptyl, octyl, nonyl, decyl, etc.), branched alkyl groups (isopropyl, tert-butyl, isobutyl, etc.), cycloalkyl groups (e.g. cyclopropyl, cyclobutyl, cyclopentyl, cyclohexyl, cycloheptyl, cyclooctyl, cyclopentenyl, cyclohexenyl, cycloheptenyl, and adamantyl), alkyl-substituted cycloalkyl groups, and cycloalkyl-substituted alkyl groups.

In certain embodiments, preferred cycloalkyl groups have 3 to 8 carbon atoms in their ring structure, more preferably 5 or 6 carbon atoms in their ring structure.

The term “C₁₋₆ alkyl” includes alkyl groups containing 1 to 6 carbon atoms, for example, methyl, ethyl, n-propyl, isopropyl, n-butyl, sec-butyl, tert-butyl, n-pentyl, sec-pentyl or n-hexyl. The term “C₁₋₃ alkyl” includes alkyl groups containing 1 to 3 carbon atoms, and specifically refers to methyl, ethyl, n-propyl and isopropyl.

In addition, the term “alkyl” also includes “unsubstituted alkyl” and “substituted alkyl”, the latter of which refers to an alkyl group in which hydrogen on one or more carbons of the hydrocarbon backbone is replaced by a substituent. Said substituent may include: alkenyl, alkynyl, halogen, hydroxyl, alkylcarbonyloxy, arylcarbonyloxy, alkoxycarbonyloxy, aryloxycarbonyloxy, hydroxycarbonyl, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonate, cyano, amino (including alkylamino, dialkylamino, arylamino, diarylamino and alkylarylamino), acylamino (including alkylcarbonylamino, arylcarbonylamino, carbamoyl and ureido), amidino, imino, mercapto, alkylthio, arylthio, hydroxythiocarbonyl, sulfate, alkylsulfinyl, sulfonic acid, sulfamoyl, sulfonamido, nitro, trifluoromethyl, cyano, azido, heterocyclic group, alkylaryl or aromatic group or heteroaromatic group.

As used herein, “heterocyclic ring” or “heterocyclic” includes any (saturated, unsaturated or aromatic) ring structures that contain at least one ring heteroatom (e.g., nitrogen, oxygen, or sulfur atom). Heterocyclic rings include heterocycloalkyl and heteroaryl. Examples of heterocyclic groups include, but are not limited to, furanyl, pyridazine, imidazolidinyl, imidazolinyl, imidazolyl, isoquinolinyl, thiazolyl, isothiazolyl, isoxazolyl, methylenedioxyphenyl, morpholinyl, oxazolidinyl, oxazolyl, oxadiazolyl, 1,2,3-oxadiazolyl, 1,2,4-oxadiazolyl, 1,2,5-oxadiazolyl, 1,3,4-oxadiazolyl, 1,2,4-oxadiazol-5(4H)-one, piperazinyl, piperidinyl, piperidonyl, 4-piperidonyl, pyranyl, tetrahydropyran, pyrazinyl, pyrazolidinyl, pyrazolinyl, pyrazolyl, pyridazinyl, pyridyl, pyrimidinyl, pyrrolidinyl, pyrrolinyl, 2H-pyrrolyl, pyrrolyl, tetrahydrofuranyl, tetrazolyl, thiazolyl, thienyl, tetrahydrothiophene, 1,2,3-triazolyl, 1,2,4-triazolyl, 1,2,5-triazolyl, 1,3,4-triazolyl, oxetane, and azetidine.

The term “aryl” or “aromatic ring” includes 5 and 6 membered monocyclic aromatic groups which may contain 0-4 heteroatoms, such as benzene, phenyl, pyrrole, furan, thiophene, thiazole, isothiazole, imidazole, triazole, tetrazole, pyrazole, oxazole, isoxazole, pyridine, pyrazine, pyridazine and pyrimidine, and the like. In addition, the term “aryl” also includes polycyclic aryl groups, such as tricyclic aryl groups and bicyclic aryl groups, such as naphthalene, benzoxazole, benzodiazole, benzothiazole, benzimidazole, benzothiophene, methylenedioxyphenyl, quinoline, isoquinoline, naphthyridine, indole, benzofuran, purine, benzofuran, deazapurine or indolizine.

Aryl groups having heteroatoms are also referred to as “aryl heterocyclic ring,” “heterocyclic ring,” “heteroaryl,” or “heteroaromatics”, wherein the heteroatoms are independently selected from nitrogen, oxygen, and sulfur, in which the nitrogen atoms may or may not be substituted, and the nitrogen and sulfur heteroatoms may be optionally oxidized (i.e., N→O and S(O)ₚ, wherein p=1 or 2), and the total number of sulfur and oxygen atoms in the aromatic heterocyclic ring does not exceed 1.

Typical heteroaryl groups include 2-thienyl or 3-thienyl; 2-furyl or 3-furyl; 2-pyrrolyl or 3-pyrrolyl; 2-imidazolyl, 4-imidazolyl or 5-imidazolyl; 3-pyrazolyl, 4-pyrazolyl or 5-pyrazolyl; 2-thiazolyl, 4-thiazolyl or 5-thiazolyl; 3-isothiazolyl, 4-isothiazolyl or 5-isothiazolyl; 2-oxazolyl, 4-oxazolyl or 5-oxazolyl; 3-isoxazolyl, 4-isoxazolyl or 5-isoxazolyl; 3-1,2,4-triazolyl or 5-1,2,4-triazolyl; 4-1,2,3-triazolyl or 5-1,2,3-triazolyl; tetrazolyl; 2-pyridyl, 3-pyridyl or 4-pyridyl; 3-pyridazinyl or 4-pyridazinyl; 3-pyrazinyl, 4-pyrazinyl or 5-pyrazinyl; 2-pyrazinyl; 2-pyrimidinyl, 4-pyrimidinyl or 5-pyrimidinyl.

The aromatic ring of an “aryl” or “heteroaryl” group may be substituted at one or more ring positions with substituents described above, such as halogen, hydroxy, alkoxy, alkylcarbonyloxy, arylcarbonyloxy, alkoxycarbonyloxy, aryloxycarbonyloxy, hydroxycarbonyl, alkylcarbonyl, alkylaminocarbonyl, arylalkylaminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonate, cyano, amino (including alkylamino, dialkylamino, arylamino, diarylamino and alkylarylamino), acylamino (including alkylcarbonylamino, arylcarbonylamino, carbamoyl and ureido), amidino, imino, mercapto, alkylthio, arylthio, hydroxylthiocarbonyl, sulfate, alkylsulfinyl, sulfonate, sulfamoyl, sulfonamido, nitro, trifluoromethyl, cyano, azido, heterocyclyl, alkylaryl, or an aromatic group or a heteroaromatic group, wherein the aryl group can also be fused or bridged with a non-aromatic alicyclic or heterocyclic ring to form a polycyclic ring (e.g. tetrahydronaphthalene).

The terms “fused bicyclic ring” and “fused bicyclyl” are used interchangeably herein to refer to a monovalent or polyvalent saturated or partially unsaturated bridged ring system. Said bridged ring system refers to a bicyclic ring system. The term “bridged ring” refers to any two rings that share two atoms that may or may not be directly connected.

The term “fused heterobicyclic group” means a saturated or partially unsaturated fused ring system, in which at least one ring system contains one or more heteroatoms, and wherein each ring system contains a 3-7 membered ring, i.e., contains 1-6 carbon atoms and 1-3 heteroatoms selected from N, O, P, and S. In this context, S or P is optionally substituted with one or more oxygen atom(s) to obtain groups such as SO, SO₂, PO, and PO₂, and said fused heterobicyclic group may be substituted or unsubstituted.

The term “C₆₋₁₀ aryl” includes aryl groups containing 6 to 10 carbon atoms.

The term “C₁₋₉ heteroaryl” includes heteroaryl groups containing 1 to 9 carbon atoms.

The term “C₆₋₁₂ fused bicyclic group” includes fused bicyclic groups containing 6 to 12 carbon atoms.

The term “C₅₋₁₂ fused heterobicyclic group” includes heterobicyclic groups containing 5 to 12 carbon atoms.

The term “alkenyl” includes unsaturated aliphatic groups similar to the alkyl groups described above in length and possible substitutions, but it contains at least one double bond.

For example, the term “alkenyl” includes linear alkenyl groups (e.g., vinyl, propenyl, butenyl, pentenyl, hexenyl, heptenyl, octenyl, nonenyl, decenyl, etc.), branched alkenyl, cycloalkenyl (e.g., cyclopropenyl, cyclopentenyl, cyclohexenyl, cycloheptenyl, cyclooctenyl), alkyl- or alkenyl-substituted cycloalkenyl, and cycloalkyl- or cycloalkenyl-substituted alkenyl. The term “alkenyl” also includes alkenyl groups containing oxygen, nitrogen, sulfur or phosphorus atoms that substitute one or more carbons of the hydrocarbon backbone. In certain embodiments, a linear or branched alkenyl group has 6 or less carbon atoms in its backbone (e.g.: C₂₋₆ linear alkenyl, C₃₋₆ branched alkenyl). The term C₂₋₆ alkenyl includes alkenyl groups containing 2 to 6 carbon atoms.

In addition, the term “alkenyl” includes “unsubstituted alkenyl” and “substituted alkenyl,” the latter of which refers to an alkenyl group in which hydrogen on one or more carbons of the hydrocarbon backbone has been replaced by a substituent. Said substituent may include, e.g., alkyl, alkynyl, halogen, hydroxyl, alkylcarbonyloxy, arylcarbonyloxy, alkoxycarbonyloxy, aryloxycarbonyloxy, hydroxycarbonyl, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonate, cyano, amino (including alkylamino, dialkylamino, arylamino, diarylamino and alkylarylamino), acylamino (including alkylcarbonylamino, arylcarbonylamino, carbamoyl and ureido), amidino, imino, mercapto, alkylthio, arylthio, hydroxythiocarbonyl, sulfate, alkylsulfinyl, sulfonic group, sulfamoyl, sulfonamido, nitro, trifluoromethyl, cyano, azido, heterocyclic group, alkylaryl or aromatic group.

The term “alkynyl” includes unsaturated aliphatic groups similar to the alkyl groups described above in length and possible substitution, but it contains at least one triple bond.

For example, the term “alkynyl” includes linear alkynyl groups (e.g. ethynyl, propynyl, butynyl, pentynyl, hexynyl, heptynyl, octynyl, nonynyl, decynyl, etc.), branched alkynyl and cycloalkyl- or cycloalkenyl-substituted alkynyl groups. The term “alkynyl” also includes alkynyl groups containing oxygen, nitrogen, sulfur or phosphorus atoms that replace one or more carbons of the hydrocarbon backbone. In certain embodiments, a linear or branched alkynyl group have 6 or less carbon atoms in the backbone (e.g., C₂-C₆ linear alkynyl group, C₃-C₆ branched alkynyl group). The term “C₂-C₆ alkynyl” refers to alkynyl groups containing 2-6 carbon atoms.

Additionally, the term alkynyl includes “unsubstituted alkynyl” and “substituted alkynyl,” the latter of which refers to an alkynyl group having a substituent that replaces hydrogen on one or more carbons in the hydrocarbon backbone. Said substituents may include, for example, alkyl, alkynyl, halogen, hydroxy, alkylcarbonyloxy, arylcarbonyloxy, alkoxycarbonyloxy, aryloxycarbonyloxy, hydroxycarbonyl, alkylcarbonyl, arylcarbonyl, alkoxycarbonyl, aminocarbonyl, alkylaminocarbonyl, dialkylaminocarbonyl, alkylthiocarbonyl, alkoxy, phosphate, —P(O)(O—)₂ (phosphonato), —PH(O)(O—) (phosphinato), cyano, amino (including alkylamino, dialkylamino, arylamino, diarylamino and alkylarylamino), acylamino (including alkylcarbonylamino, arylcarbonylamino, carbamoyl and ureido), amidino, imino, mercapto, alkylthio, arylthio, hydroxythiocarbonyl, sulfate, alkylsulfinyl, —SO₂O— (sulfonato), sulfamoyl, sulfonamido, nitro, trifluoromethyl, cyano, azido, heterocyclic group, alkylaryl, or aromatic group or heteroaromatic group.

The term “C₂₋₆ alkynyl” includes alkynyl groups containing 2 to 6 carbon atoms.

The term “alkoxy” includes substituted and unsubstituted alkyl groups covalently attached to an oxygen atom. Examples of alkoxy groups include methoxy, ethoxy, isopropoxy, propoxy, butoxy and pentoxy. Examples of substituted alkoxy groups include haloalkoxy groups. Alkoxy can be substituted with the following substituents: alkenyl, alkynyl, halogen, hydroxy, alkylcarbonyloxy, arylcarbonyloxy, alkoxycarbonyloxy, aryl oxycarbonyloxy, hydroxycarbonyl, alkylcarbonyl, arylcarbonyl, alkoxycarbonyl, aminocarbonyl, alkylaminocarbonyl, dialkylaminocarbonyl, alkylthiocarbonyl, phosphate, cyano, amino (including alkylamino, dialkylamino, arylamino, diarylamino and alkylarylamino), acylamino (including alkylcarbonylamino, arylcarbonylamino, carbamoyl and ureido), amidino, imino, mercapto, alkylthio, arylthio, hydroxythiocarbonyl, alkylsulfinyl, sulfonic group, sulfamoyl, sulfonamido, nitro, trifluoromethyl, cyano, azido, heterocyclic group, alkylaryl or aromatic group.

The term “cycloalkyl” refers to a monovalent or polyvalent saturated monocyclic, bicyclic or tricyclic ring system containing 3 to 12 carbon atoms, wherein the monocyclic, bicyclic or tricyclic rings do not contain aromatic rings. The cycloalkyl group may independently be unsubstituted or substituted with one or more substituents described herein.

The term “hydroxy” includes groups having —OH or —O—.

The term “halogen” includes fluorine, chlorine, bromine, iodine, and the like.

As used herein, the term “substituted” means that any one or more hydrogen atoms on the designated atom are replaced by a substituent selected from a designated group, resulting in a stable compound as the result of the substitution. When the substituent is an oxo group or keto group (i.e., ═O), 2 hydrogen atoms on the atom are substituted. A ketone substituent does not exist on an aromatic ring.

The pharmaceutically acceptable salt of the present invention include pharmaceutically acceptable acid addition salt and pharmaceutically acceptable base addition salt.

“Pharmaceutically acceptable acid addition salt” refers to a salt formed with inorganic or organic acid, which retains the biological effectiveness of the free base without other side effects. Inorganic acid salts include, but are not limited to, hydrochloride, hydrobromide, sulfate, phosphate, nitrate, and the like. Organic acid salts include, but are not limited to, formate, acetate, propionate, 2,2-dichloroacetate, butyrate, caproate, caprylate, caprate, undecenoate, glycolate, gluconate, lactate, sebacate, adipate, glutarate, malonate, oxalate, maleate, succinate, fumarate, tartrate, citrate, palmitate, stearate, oleate, cinnamate, malate, laurate, glutamate, pyroglutamate, aspartate, benzoate, mesylate, besylate, p-toluenesulfonate, alginate, ascorbate, salicylate, 4-aminosalicylate, naphthalene disulfonate, and the like.

“Pharmaceutically acceptable base addition salts” refers to a salt formed with inorganic or organic base, which retains the biological effectiveness of the free acid without other adverse effects. Salts derived from inorganic base include, but are not limited to, potassium salt, sodium salt, lithium salt, ammonium salt, calcium salt, magnesium salt, iron salt, zinc salt, copper salt, manganese salt, aluminum salt, and the like. Preferred inorganic salts are ammonium salt, sodium salt, potassium salt, calcium salt and magnesium salt. Salts derived from organic base include, but are not limited to, the following salts: primary, secondary and tertiary amines, substituted amines, including natural substituted amines, cyclic amines, and basic ion exchange resins, such as ammonia, trimethylamine, isopropylamine, diethylamine, triethylamine, tripropylamine, ethanolamine, diethanolamine, triethanolamine, dimethylethanolamine, 2-dimethylaminoethanol, 2-diethylaminoethanol, bicyclic hexylamine, lysine, arginine, histidine, caffeine, procaine, betaine, choline, ethylenediamine, glucosamine, methylglucamine, theobromine, purine, piperidine, piperazine, N-ethylpiperidine, polyamine resin, etc. Preferred organic bases include isopropylamine, trimethylamine, diethylamine, ethanolamine, dicyclohexylamine, choline and caffeine.

When the compound of the present invention contains at least one salt-forming nitrogen atom in the molecule, it can be converted into a corresponding salt by reacting with a corresponding organic or inorganic acid in an organic solvent such as acetonitrile and tetrahydrofuran. Typical organic acids are oxalic acid, tartaric acid, maleic acid, succinic acid, methanesulfonic acid, benzoic acid, benzenesulfonic acid, toluenesulfonic acid, sulfamic acid, citric acid, glutamic acid, pyroglutamic acid, aspartic acid, glucuronic acid, naphthalenesulfonic acid, glutaric acid, acetic acid, trifluoroacetic acid, malic acid, fumaric acid, salicylic acid, 4-aminosalicylic acid, lactic acid, palmitic acid, stearic acid, lauric acid, cinnamic acid, alginic acid, and ascorbic acid. Typical inorganic acids are nitric acid, hydrochloric acid, sulfuric acid, and phosphoric acid.

When the compounds of the present invention have one or more asymmetric carbon atoms, they can exist in the following forms: optically pure enantiomer, pure diastereomer, mixture of enantiomers, mixture of diastereoisomers, racemic mixture of enantiomers, racemate or mixture of racemates. All possible isomers, stereoisomers and mixtures of compounds of formula (II) are also within the scope of the present invention.

The present invention also provides a pharmaceutical composition comprising at least one compound described above and optionally one or more pharmaceutically acceptable carriers and/or diluents.

The pharmaceutical compositions provided by the present invention can be prepared in any forms, such as granules, powders, tablets, coated tablets, capsules, pills, syrups, drops, solutions, suspensions and emulsions, or sustained release formulations of active ingredients, wherein examples of capsules include hard or soft gelatin capsules, and the granules and powders may be in non-effervescent or effervescent form.

The pharmaceutical compositions of the present invention may further comprise one or more pharmaceutically or physiologically acceptable carriers which will be suitable for formulating, and so as to be suitable for administration. For example, a pharmaceutically or physiologically acceptable carrier can be saline, hot-compressed water, Ringer's solution, buffered saline, glucose, maltodextrin, glycerol, ethanol, and mixtures thereof. The pharmaceutical composition of the present invention may also include a pharmaceutically or physiologically acceptable additive, such as diluent, lubricant, binder, glidant, disintegrant, sweetening agent, flavoring agent, wetting agent, dispersing agent, surfactant, solvent, coating agent, foaming agent, or fragrance.

Examples of diluents that can be used include, but are not limited to, lactose, sucrose, starch, kaolin, salt, mannitol, and dicalcium phosphate. Examples of lubricants include, but are not limited to, talc, starch, magnesium stearate or calcium stearate, lycopodium and stearic acid. Examples of binders include, but are not limited to, microcrystalline cellulose, gum tragacanth, glucose solution, acacia mucilage, gelatin solution, sucrose, and starch paste. Examples of glidants include, but are not limited to, colloidal silica. Examples of disintegrants include, but are not limited to, croscarmellose sodium, starch sodium glycollate, alginic acid, corn starch, potato starch, bentonite, methylcellulose, agar, and carboxymethylcellulose. Examples of sweetening agents include, but are not limited to, sucrose, lactose, mannitol, and artificial sweetening agents, such as sodium cyclamate and saccharin, and any amount of spray-dried flavoring agents. Examples of flavoring agents include, but are not limited to, natural flavoring agents extracted from plants such as fruits, and compounds with good-tastings, such as, but not limited to, peppermint and methyl salicylate. Examples of wetting agents include, but are not limited to, propylene glycol mono-stearates, sorbitan monooleate, diethylene glycol monolaurate and polyoxyethylene lauryl ether.

The pharmaceutical compositions of the present invention can be administered according to conventional methods by various routes, including oral, intravenous, intraarterial, intraperitoneal, intrathoracic, transdermal, nasal, inhalation, rectal, ocular and subcutaneous administration.

Pharmaceutically acceptable carriers that optionally added to the pharmaceutical composition of the present invention are one or more of water, alcohol, honey, mannitol, sorbitol, dextrin, lactose, caramel, gelatin, calcium sulfate, magnesium stearate, talc, kaolin, glycerin, tween, agar, calcium carbonate, calcium bicarbonate, surfactant, cyclodextrin and derivatives thereof, phospholipids, phosphates, starch and derivatives thereof, silicon derivatives, celluloses and derivatives thereof, pyrrolidones, polyethylene glycols, acrylic resins, phthalates, acrylic copolymers, and trimellitates.

It has been verified by pharmacological experiments that the compound or pharmaceutical composition provided by the present invention can treat cancer, myeloproliferative diseases or autoimmune diseases through PD-1. The said cancer is nasopharyngeal cancer, lung cancer, breast cancer, prostate cancer, ovarian cancer, cervical cancer, oesophageal carcinoma, esophageal cancer, stomach cancer, liver cancer, colorectal cancer, rectal cancer, skin cancer, epithelial cell cancer, bladder cancer, nasopharyngeal cancer, blood cancer or bone cancer. The said myeloproliferative disease is leukemia. The said autoimmune disease is inflammatory bowel disease, autoimmune encephalomyelitis or multiple sclerosis.

The general dosage range of the compound provided by the present invention is about 0.001 mg/kg to 1000 mg/kg per day, preferably about 0.01 mg/kg to 100 mg/kg per day, more preferably about 0.1 mg/kg to 20 mg/kg per day. The dosage range of the pharmaceutical composition is calculated based on the amount of the above-mentioned compounds contained therein.

### DETAILED DESCRIPTION OF THE INVENTION

### Example 1

### N-(2-chloro-3′-(4-(((2-hydroxyethyl)amino)methyl)-1-methyl-1H-imidazole-2-carboxamido)-2′-methyl-[1,1′-biphenyl]-3-yl)-5-(((2-hydroxyethyl)amino)methyl)picolinamide

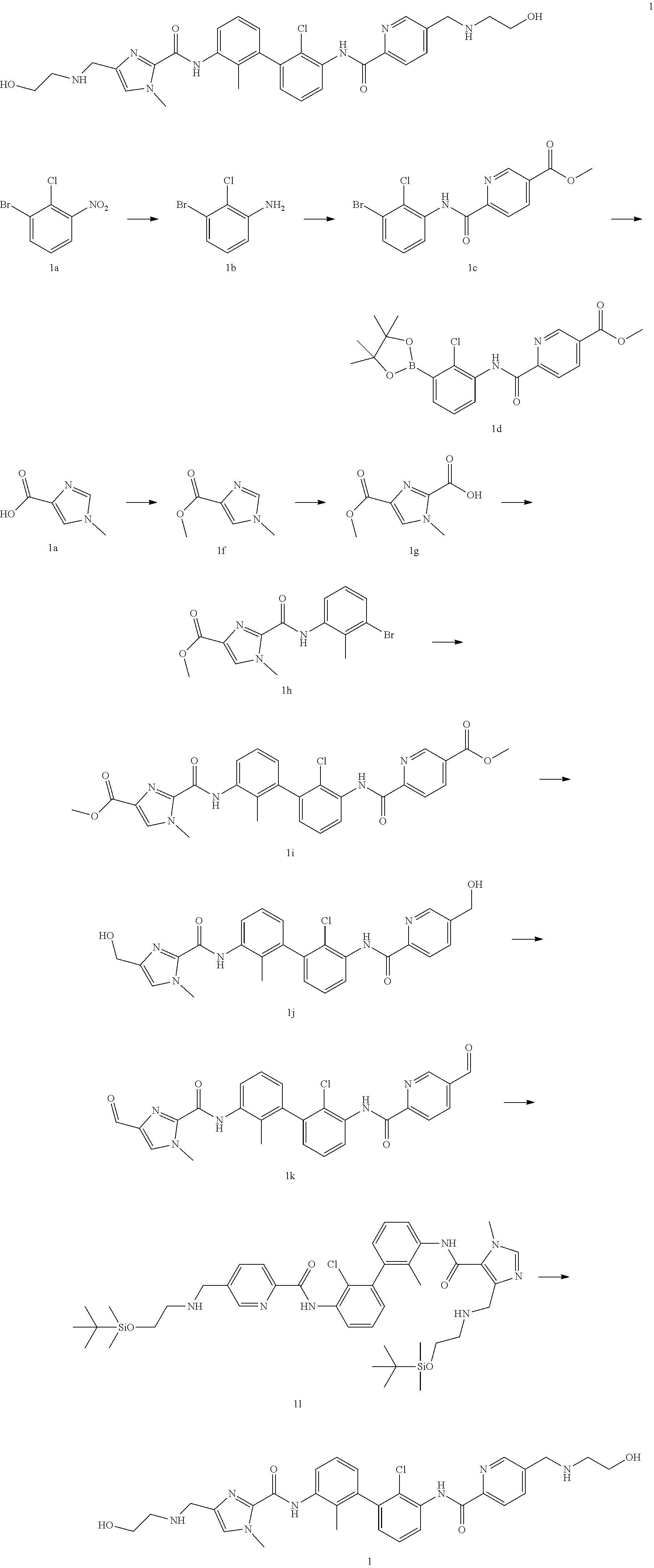

### Step 1 3-bromo-2-chloroaniline 1b

Compound 1a (10 g, 42.372 mol), zinc powder (22 g, 338.98 mmol), and ammonium chloride (17.96 g, 338.98 mmol) were dissolved in ethanol (50 mL), THF (50 mL) and H₂O (25 mL), heated to 60° C. and stirred for 6 h until the reaction was finished. The reaction solution was filtered, dried by rotary evaporation, and extracted with water and ethyl acetate. The organic phase was washed with saturated brine, dried over anhydrous sodium sulfate, filtered, concentrated, and purified by column chromatography to obtain the title compound 1b (7 g, 33.98 mmol) with a yield of 80%. MS m/z (ESI): 208[M+1]⁺.

### Step 2 methyl 6-((3-bromo-2-chlorophenyl)carbamoyl)nicotinate 1c

Compound 1b (2.06 g, 10 mmol) and 5-(methoxycarbonyl)picolinic acid (2.715 g, 15 mmol) were dissolved in N,N-dimethylformamide (50 mL). HATU (7.6 g, 20 mmol) and DIPEA (5.16 g, 40 mmol) were added, and reacted at room temperature for 24 h until the reaction was finished. The reaction solution was washed with water, extracted three times with dichloromethane, washed with saturated brine, dried over anhydrous sodium sulfate, filtered, concentrated and purified by column chromatography to obtain the title compound 1c (2 g, 5.42 mmol) with a yield of 54%. MS m/z (ESI): 371 [M+H]⁺.

### Step 3 methyl 6-((2-chloro-3-(4,4,5,5-tetramethyl-1,3,2-dioxaboran-2-yl)phenyl)carbamoyl)nicotinate 1 d

Compound 1c (1.8 g, 4.878 mmol) and diboron pinacol ester (2.478 g, 9.756 mmol) were dissolved in dioxane (50 mL). [1,1′-Bis(diphenylphosphino)ferrocene]palladium dichloride (0.356 g, 0.4878 mmol) and potassium acetate (1.434 g, 14.634 mmol) were added, and the mixture was heated to 100° C. under argon protection and stirred for 4 h until the reaction was finished. The reaction solution was added with water and ethyl acetate for extraction. The organic phase was washed with saturated brine, dried over anhydrous sodium sulfate, filtered, concentrated and purified by column chromatography to obtain the title compound 1d (1.6 g, 3.846 mmol) with a yield of 78.8%. MS m/z (ESI) 417 [M+1]⁺.

### Step 4 methyl 1-methyl-1H-imidazole-4-carboxylate 1 f

Compound 1e (5 g, 39.68 mmol) was dissolved in methanol (100 mL), and then thionyl chloride (9.44 g, 79.36 mmol) was slowly added dropwise. The mixture was heated under reflux for 6 h until the reaction was finished. The reaction solution was dried by rotary evaporation, added with saturated sodium bicarbonate solution, extracted three times with ethyl acetate, washed with saturated brine, dried over anhydrous sodium sulfate, filtered, concentrated and purified by column chromatography to obtain the title compound 1 f (3.6 g, 0.4 mmol) with a yield of 63.7%. MS m/z (ESI): 141.2 [M+H]⁺.

### Step 5 4-(methoxycarbonyl)-1-methyl-1H-imidazole-2-carboxylic acid 1 g

Compound 1f (1 g, 7.14 mmol) was dissolved in tetrahydrofuran (40 mL). Under argon protection, LDA (14.3 mL, 14.3 mmol) was slowly added dropwise in a dry ice acetone bath, and was stirred for 1 h. Then, the protection was replaced with a CO₂ balloon. The reaction was carried out at −78° C. for 1 h, slowly warmed to room temperature, and stirred overnight. The reaction was quenched by adding water, adjusted to pH=4-5 with dilute hydrochloric acid, extracted three times with ethyl acetate, washed with saturated brine, dried over anhydrous sodium sulfate, filtered, and concentrated to obtain the title compound 1 g (560 mg, 3.04 mmol) with a yield of 42.6%. MS m/z (ESI): 185.1[M+1]⁺.

### Step 6 methyl 2-((3-bromo-2-methylphenyl)carbamoyl)-1-methyl-1H-imidazole-4-carboxylate 1h

Compound 1 g (560 mg, 3.043 mmol) and 3-bromo-2-methylaniline (849 mg, 4.565 mmol) were dissolved in N,N-dimethylformamide (20 mL). HATU (2.312 g, 6.086 mmol) and DIPEA (1.57 g, 12.172 mmol) were added, and reacted at room temperature for 24 h until the reaction was finished. The reaction solution was washed with water, extracted three times with dichloromethane, washed with saturated brine, dried over anhydrous sodium sulfate, filtered, concentrated and purified by column chromatography to obtain the title compound 1 h (500 mg, 1.42 mmol) with a yield of 46%. MS m/z (ESI): 352.1 [M+H]⁺.

### Step 7 methyl 6-((2-chloro-3′-(4-(methoxycarbonyl)-1-methyl-1H-imidazole-2-carboxamido)-2′-methyl-[1,1′-biphenyl]-3-yl)carbamate)nicotinate 1i

Compound 1 h (500 mg, 1.42 mmol) and compound 1 d (886 mg, 2.13 mmol) were dissolved in 1,4-dioxane (50 mL) and water (15 mL). [1,1′-Bis(diphenylphosphino)ferrocene]palladium dichloride (104 mg, 0.142 mmol) and potassium phosphate (903 mg, 4.26 mmol) were added. The mixture was heated to 100° C. under argon protection and stirred for 5 h until the reaction was finished. The reaction solution was added with water and ethyl acetate for extraction. The organic phase was washed with saturated brine, dried over anhydrous sodium sulfate, filtered, concentrated and purified by column chromatography to obtain the title compound 1i (300 mg, 0.534 mmol) with a yield of 37%. MS m/z (ESI): 562.3 [M+H]⁺.

### Step 8 N-(2-chloro-3′-(4-(hydroxymethyl)-1-methyl-1H-imidazole-2-carboxamido)-2′-methyl-[1,1′-biphenyl]-3-yl)-5-(hydroxymethyl)picolinamide 1j

Compound 1 i (200 mg, 0.3558 mmol) was dissolved in ethanol (20 mL). Sodium borohydride (134.5 mg, 3.558 mmol) was added under argon protection in an ice bath. The mixture was slowly warmed to room temperature and stirred overnight until the reaction was finished. The reaction solution was quenched with dilute hydrochloric acid, added with saturated sodium bicarbonate, extracted three times with ethyl acetate, washed with saturated brine, dried over anhydrous sodium sulfate, filtered, concentrated and purified by column chromatography to obtain the title compound 1j (100 mg, 0.198 mmol) with a yield of 55%. MS m/z (ESI): 506.3 [M+H]⁺

### Step 9 N-(2-chloro-3′-(4-formyl-1-methyl-1H-imidazole-2-carboxamido)-2′-methyl-[1,1′-biphenyl]-3-yl)-5-formylpyridinoline 1k

Compound 1j (100 mg, 0.198 mmol) was dissolved in dichloromethane (20 mL). Dess Martin reagent (252 mg, 0.594 mmol) was added, and was reacted for 2 h at room temperature until the reaction was finished. The reaction solution was quenched with sodium sulfite, extracted three times with dichloromethane, washed with saturated brine, dried over anhydrous sodium sulfate, filtered, concentrated and purified by plate chromatography to obtain the title compound 1 k (65 mg, 0.1297 mmol) with a yield of 65%. MS m/z (ESI): 502.3 [M+H]⁺.

### Step 10 5-((((2-((tert-butyldimethylsilyl)oxy)ethyl)amino)methyl)-N-(3 ‘-(4-(((((2-((tert-butyldimethylsilylmethoxy)ethyl)amino]methyl)-1-methyl-1H-imidazole-5-carboxamido)-2-chloro-2’-methyl-[1,1′-biphenyl]-3-yl)picolinamide 1l

Compound 1k (65 mg, 0.1297 mmol) was dissolved in dichloromethane (20 mL), and acetic acid (16 mg, 0.2594 mmol) and 2-((tert-butyldimethylsilyl)oxy)ethan-1-amine (89 mg, 0.5189 mmol) were added. The reaction solution was stirred at room temperature for 30 min, then added with sodium borohydride acetate (165 mg, 0.7782 mmol), and stirred at room temperature for 13 h until the reaction was finished. The reaction solution was quenched with water, extracted three times with dichloromethane, washed with saturated brine, dried over anhydrous sodium sulfate, filtered, concentrated and purified by plate chromatography to obtain the title compound 11 l mmol) with a yield of 70%. MS m/z (ESI): 820.7[M+1]⁺.

### Step 11 N-(2-chloro-3′-(4-(((2-hydroxyethyl)amino)methyl)-1-methyl-1H-imidazole-2-carboxamido)-2′-methyl-[1,1′-biphenyl]-3-yl)-5-(((2-hydroxyethyl)amino)methyl)picolinamide 1

Compound 1l (75 mg, 0.091 mmol) was dissolved in tetrahydrofuran (10 mL). TBAF/THF (0.73 mL, 0.73 mmol) was added. The reaction solution was stirred at room temperature for 3 h until the reaction was finished. The resultant was concentrated and purified by Prep-HPLC to obtain the title compound 1 (23 mg, 0.03885 mmol) with a yield of 42%. MS m/z (ESI): 592.4[M+1]⁺

¹H NMR (400 MHz, DMSO-d₆) δ 10.70 (s, 1H), 8.74 (s, 1H), 8.42 (d, J=8.3 Hz, 1H), 8.21 (d, J=8.1 Hz, 1H), 8.12 (d, J=7.8 Hz, 2H), 7.76 (s, 1H), 7.49 (q, J=7.8 Hz, 2H), 7.28 (t, J=7.8 Hz, 1H), 7.10 (d, J=7.5 Hz, 1H), 7.01 (d, J=7.5 Hz, 1H), 4.73 (s, 2H), 4.08 (s, 2H), 3.97 (s, 2H), 3.81 (s, 3H), 3.55 (t, J=5.6 Hz, 2H), 3.47 (s, 2H), 2.79 (t, J=5.2 Hz, 2H), 2.68 (s, 2H), 1.97 (s, 3H).

### Example 2

### N-(2′-chloro-3′-(5-((((2-hydroxyethyl)amino)methyl)picolinamido)-2-methyl-[1,1′-biphenyl]-3-yl)-4-(((2-hydroxyethyl)amino)methyl)thiazole-2-carboxamide

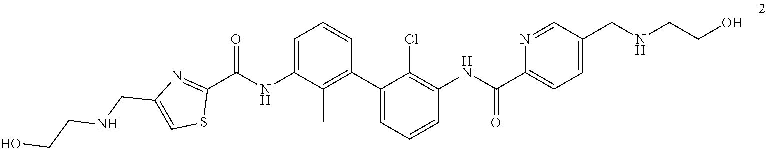 [SMILES: Cc1c(NC(=O)c2nc(CNCCO)cs2)cccc1-c1cccc(NC(=O)c2ccc(CNCCO)cn2)c1Cl]

A synthetic method similar to that of Example 1 was used to prepare the title product 2.

MS m/z (ESI): 595.4[M+1]⁺

¹H NMR (400 MHz, DMSO-d₆) δ 10.70 (s, 1H), 10.29 (s, 1H), 8.66 (d, J=2.0 Hz, 1H), 8.46 (dd, J=8.2, 1.6 Hz, 1H), 8.15 (d, J=8.0 Hz, 1H), 8.02 (dd, J=8.0, 2.0 Hz, 1H), 7.79 (s, 1H), 7.55-7.44 (m, 2H), 7.30 (t, J=7.8 Hz, 1H), 7.08 (ddd, J=6.5, 5.0, 1.4 Hz, 2H), 4.47 (q, J=5.8 Hz, 2H), 3.88 (s, 2H), 3.82 (s, 2H), 3.44 (dt, J=8.8, 5.4 Hz, 4H), 2.62 (t, J=5.7 Hz, 2H), 2.54 (t, J=5.8 Hz, 2H), 1.97 (s, 3H).

### Example 3

### (S)—N-(5-(3-(1,5-dimethyl-4,5,6,7-tetrahydro-1H-imidazo[4,5-c]pyridine-2-carboxamido)-2-methylphenyl)-4-methylpyridin-3-yl)-5-(2-hydroxypropyl)-1-methyl-4,5,6,7-tetrahydro-1H-imidazo[4,5-c]pyridine-2-carboxamide

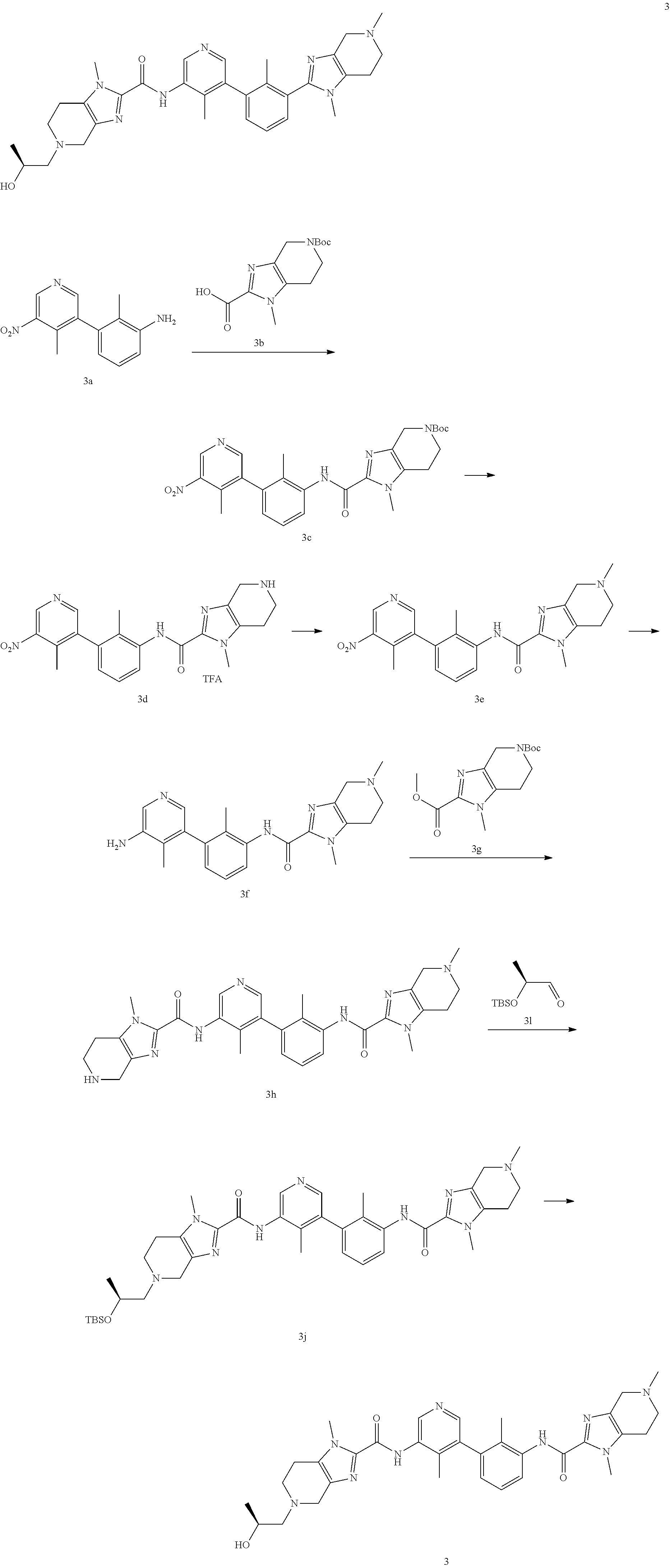

### Step 1 tert-butyl 1-methyl-2-((2-methyl-3-(4-methyl-5-nitropyridin-3-yl)phenyl)carbamoyl)-1,4,6,7-tetrahydro-5H-imidazo[4,5-c]pyridine-5-carboxylate 3c

Compound 3a (1.02 g, 4.21 mmol) and compound 3b (1.18 g, 4.21 mmol) were dissolved in DMSO (20 mL). HATU (1.75 g, 4.63 mmol) and TEA (1.27 g, 12.6 mmol) were added. The atmosphere was replaced with nitrogen for three times. The reaction was stirred at room temperature for 20 h. The reaction solution was added with water, extracted three times with EtOAc, washed with saturated brine for several times, dried over anhydrous sodium sulfate, filtered, concentrated and purified by column chromatography to obtain the title compound 3c (1.03 g, yield 48%). MS m/z (ESI): 507.4[M+1]+.

### Step 2 1-methyl-N-(2-methyl-3-(4-methyl-5-nitropyridin-3-yl)phenyl)-4,5,6,7-tetrahydro-1H-imidazo[4,5-c]pyridine-2-carboxamide hydrochloride 3d

Compound 3c (1.00 g, 1.97 mmol) was dissolved in EtOAc (10 mL). HCl/EtOAc (2 N, 10 mL) was added. The reaction solution was stirred at room temperature for 2 h, and evaporated to dryness to obtain a crude product of the title compound 3d (730 mg), which was used in the next reaction step without purification. MS m/z (ESI): 407.3[M+1]⁺.

### Step 3 1,5-dimethyl-N-(2-methyl-3-(4-methyl-5-nitropyridin-3-yl)phenyl)-4,5,6,7-tetrahydro-1H-imidazo[4,5-c]pyridine-2-carboxamide 3e

Compound 3d (730 mg, 1.00 mmol) was dissolved in dichloromethane (40 mL) and methanol (4.0 mL), added with paraformaldehyde (1.80 g, 20.0 mmol) and subsequently sodium borohydride acetate (1.05 g, 5.00 mmol), which were stirred at room temperature for 20 h until the reaction was finished. The reaction solution was quenched with saturated aqueous NaHCO₃ solution, extracted three times with dichloromethane, dried over anhydrous sodium sulfate, filtered, concentrated and purified by column chromatography to obtain the title compound 3e (384 mg, yield 82%). MS m/z (ESI): 421.3[M+1]⁺.

### Step 4 N-(3-(5-amino-4-methylpyridin-3-yl)-2-methylphenyl)-1,5-dimethyl-4,5,6,7-tetrahydro-1H-imidazo[4,5-C]pyridine-2-carboxamide 3f

Compound 3e (350 mg, 0.83 mmol) was dissolved in EtOH (10 mL), and added with Pd/C (50 mg). Under the blowing of hydrogen, the reaction solution was stirred at 52° C. for 4 h, and filtered. The filtrate was then concentrated to obtain compound 3f (211 mg, 64% yield) as a white solid, which was used in the next reaction step without purification.

MS m/z (ESI): 391.3[M+1]⁺.

### Step 5 1,5-dimethyl-N-(2-methyl-3-(4-methyl-5-(1-methyl-4,5,6,7-tetrahydro-1H-imidazo[4,5-c]pyridine-2-carboxamido)pyridin-3-yl)phenyl)-4,5,6,7-tetrahydro-1H-imidazo[4,5-c]pyridine-2-formamide hydrochloride 3h

Compound 3f (100 mg, 0.25 mmol) and compound 3g (150 mg, 0.50 mmol) were dissolved in toluene (10 mL) and cooled to −78° C. LiHDMS (0.5 mL, 0.50 mmol) was slowly added, and reacted at room temperature overnight. The reaction solution was added with water, extracted with EtOAc, dried over anhydrous sodium sulfate, filtered, concentrated and purified by column chromatography to obtain a BOC-protected title compound (50 mg, yield 30%) as a light-colored oil. HCl/EtOAc (5.0 mL) was added. The reaction solution was stirred at room temperature for 2 h, and concentrated to obtain a crude of the title compound 3h, which was directly used in the next step.

MS m/z (ESI): 554[M+1]⁺.

### Step 6 (S)-5-(2-((tert-butyldimethylsilyl)oxy)propyl)-N-(5-(3-(1,5-dimethyl-4,5,6,7-tetrahydro-1H-imidazo[4,5-c]pyridine-2-carboxamido)-2-methylphenyl)-4-methylpyridin-3-yl)-1-methyl-4,5,6,7-tetrahydro-1H-imidazo[4,5-c]pyridine-2-carboxamide 3j

Compound 3h (50 mg, 0.07 mmol) and compound 3i (39 mg, 0.21 mmol) were dissolved in dichloromethane (20 mL) and methanol (2 mL), and were added with sodium borohydride acetate (74 mg, 0.35 mmol), which were stirred overnight until the reaction was finished. The reaction solution was quenched with NaHCO₃ aqueous solution, and extracted three times with dichloromethane. The organic phases were combined and dried over anhydrous sodium sulfate, filtered, concentrated and purified by column chromatography to obtain the title compound 3j (60 mg, crude product), which was directly used in the next step.

MS m/z (ESI): 726.7[M+1]⁺.

### Step 8 (S)—N-(5-(3-(1,5-dimethyl-4,5,6,7-tetrahydro-1H-imidazo[4,5-c]pyridine-2-carboxamido)-2-methylphenyl)-4-methylpyridin-3-yl)-5-(2-hydroxypropyl)-1-methyl-4,5,6,7-tetrahydro-1H-imidazo[4,5-c]pyridine-2-carboxamide 3

Compound 3j (60 mg, 0.07 mmol) was dissolved in EtOAc (5 mL) and added with HCl/EtOAc (2N, 5 mL), which were stirred at room temperature for 5 h, adjusted to neutral pH with 15% aqueous sodium bicarbonate solution, and extracted with EA. The organic phases were combined and dried over anhydrous sodium sulfate, filtered, concentrated and purified by column chromatography to obtain the title compound 3 (8.0 mg, yield 18%).

MS M/z (ESI): 612.5[M+1]⁺

¹H NMR (400 MHz, DMSO-d₆) δ 10.00 (s, 11H), 9.77 (s, 1H), 8.61 (s, 1H), 8.11 (s, 1H), 7.65 (dd, J=8.1, 1.3 Hz, 1H), 7.28 (t, J=7.8 Hz, 1H), 6.98 (dd, J=7.6, 1.3 Hz, 1H), 5.29 (s, 1H), 4.34 (d, J=4.1 Hz, 1H), 3.83 (d, J=2.7 Hz, 5H), 3.47 (d, J=3.5 Hz, 3H), 2.79 (t, J=5.8 Hz, 2H), 2.64 (q, J=4.2 Hz, 6H), 2.40-2.33 (m, 3H), 1.94 (t, J=6.5 Hz, 7H), 1.43 (d, J=7.7 Hz, 1H), 1.04 (d, J=6.1 Hz. 3H).

### Example 4

### (R)-1-(((2-(2,2′-dimethyl-3′-(5-methyl-4,5,6,7-tetrahydrothiazolo[5,4-c]pyridine-2-carboxamido))-[1,1′-biphenyl]-3-yl)oxazolo[5,4-b]pyridin-6-yl)methyl)pyrrolidine-3-carboxylic acid

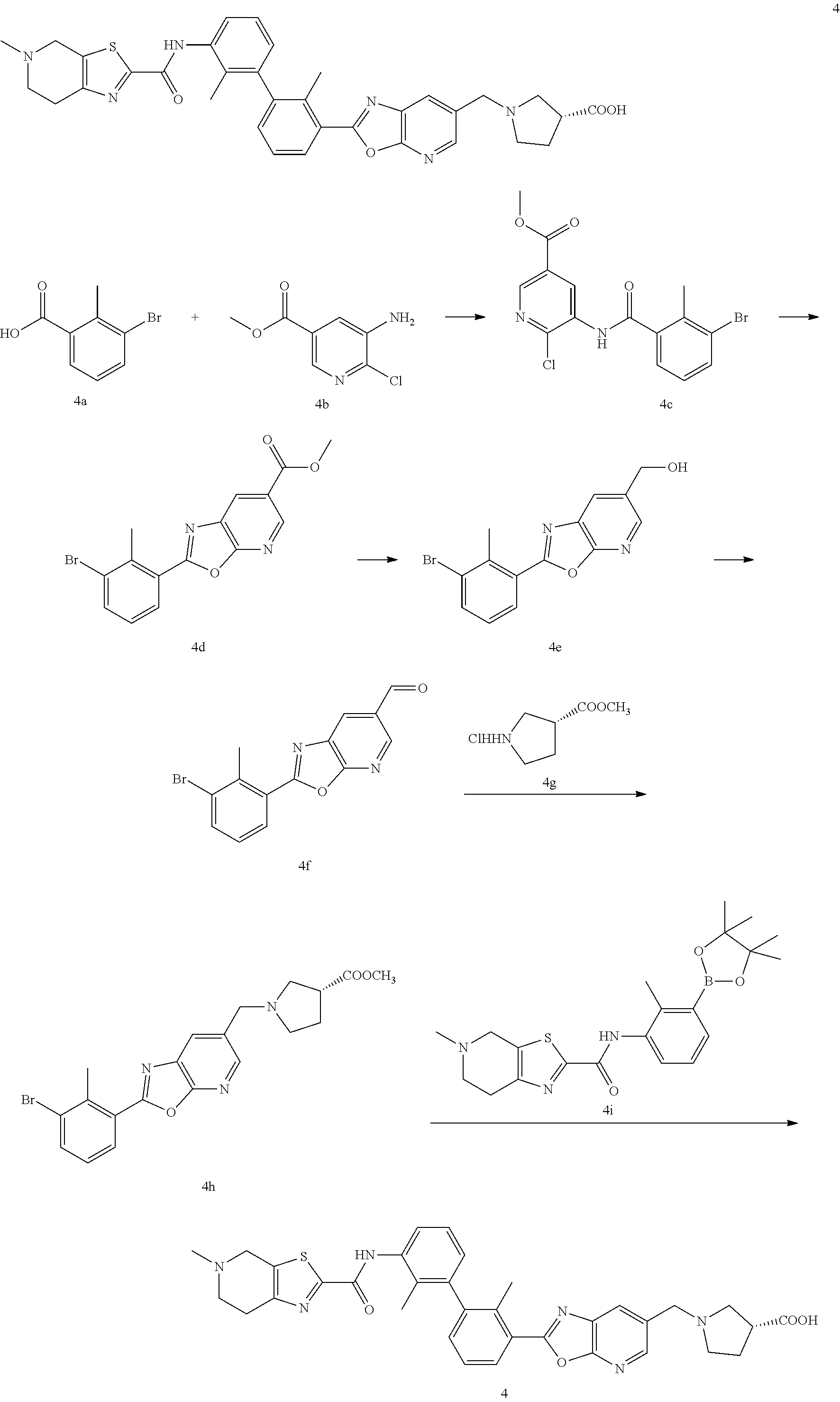

### Step 1 methyl 5-(3-bromo-2-methylbenzamido)-6-chloronicotinate 4c

Compound 4a (6.45 g, 30 mmol) and compound 4b (5.58 g, 30 mmol) were dissolved in pyridine (30 mL), and phosphorus oxychloride (9.18 g, 60 mmol) was slowly added dropwise in an ice bath, which were stirred at room temperature for 3 h until the reaction was finished. Ice water was added, and subsequently the resultant was extracted with ethyl acetate. The organic phase was washed with saturated copper sulfate solution and saturated brine, dried over anhydrous sodium sulfate, filtered, concentrated and purified by column chromatography to obtain the title compound 4c (4 g, 10.44 mmol) with a yield of 35%.

MS m/z (ESI): 385.1[M+1]⁺.

### Step 2 methyl 2-(3-bromo-2-methylphenyl)oxazolo[5,4-b]pyridine-6-carboxylate 4d

Compound 4c (3 g, 7.832 mmol) and N,N′-dimethylethylenediamine (69 mg, 0.7832 mmol) were dissolved in toluene (50 mL), added with CuI (149 mg, 0.7832 mmol) and potassium carbonate (2.161 g, 15.664 mmol), heated to 120° C. under argon protection and stirred for 24 h until the reaction was finished. The reaction solution was added with water, extracted three times with ethyl acetate, washed with saturated brine, dried over anhydrous sodium sulfate, filtered, concentrated and purified by column chromatography to obtain the title compound 4d (1.5 g, 4.322 mmol) with a yield of 55%.

MS m/z (ESI): 347 [M+H]⁺.

### Step 3 (2-(3-bromo-2-methylphenyl)oxazolo[5,4-b]pyridin-6-yl)methanol 4e

Compound 4d (3 g, 8.645 mmol) was dissolved in dichloromethane (60 mL), cooled to −78° C. under argon protection. DIBAL-H (11.5 mL, 17.29 mmol) was then slowly added dropwise. When the addition was completed, the reaction was stirred at this temperature for 1 h, then warmed to room temperature, and quenched by adding water. After that, saturated sodium potassium tartrate was added. The resultant was then extracted with ethyl acetate. The organic phase was washed with saturated sodium carbonate and brine, dried over anhydrous sodium sulfate, filtered, and concentrated to obtain the title compound 4e (2.2 g, 6.89 mmol) with a yield of 79%.

MS m/z (ESI): 319 [M+1]⁺.

### Step 4 2-(3-bromo-2-methylphenyl)oxazolo[5,4-b]pyridine-6-formaldehyde 4f

Compound 4e (1.5 g, 4.702 mmol) was dissolved in dichloromethane (50 mL), and then Dess Martin oxidant (3.987 g, 9.404 mmol) was added, which were reacted at room temperature for two hours until the reaction was finished. Sodium thiosulfate solution was added and stirred for 1 h. The resultant was extracted three times with ethyl acetate, washed with saturated brine, dried over anhydrous sodium sulfate, filtered, concentrated and purified by column chromatography to obtain the title compound 4f (1.2 g, 3.785 mmol) with a yield of 80%.

MS m/z (ESI): 317.1 [M+H]⁺.

### Step 5 methyl (R)-1-((2-(3-bromo-2-methylphenyl)oxazolo[5,4-b]pyridin-6-yl)methyl)pyrrolidine-3-carboxylate 4h

Compound 4f (1.2 g, 3.785 mmol) and methyl (R)-pyrrolidine-3-carboxylate hydrochloride 4g (1.249 g, 7.57 mmol) were dissolved in dichloromethane (60 mL), stirred for 1 h and added with sodium triacetoxyborohydride (2.407 g, 11.356 mmol), which were stirred overnight at room temperature until the reaction was finished. The reaction solution was added with water and ethyl acetate for extraction. The organic phase was washed with saturated brine, dried over anhydrous sodium sulfate, filtered, concentrated and purified by column chromatography to obtain the title compound 4f (0.7 g, 1.627 mmol) with a yield of 43%.

MS m/z (ESI): 430.2[M+1]⁺.

### Step 6 (R)-1-(((2-(2,2′-dimethyl-3′-(5-methyl-4,5,6,7-tetrahydrothiazolo[5,4-c]pyridine-2-carboxamido))-[1,1′-biphenyl]-3-yl)oxazolo[5,4-b]pyridin-6-yl)methyl)pyrrolidine-3-carboxylic acid 4

Compound 4h (100 mg, 0.2325 mmol) and compound 4i (144 mg, 0.3488 mmol) were dissolved in tert-butanol/water (v/v=2/1) (15 mL), and 1,1′-bis(di-cyclohexylphosphine)ferrocene palladium dichloride (18 mg, 0.02325 mmol) and cesium carbonate (227 mg, 0.6975 mmol) were added, which were reacted at 100° C. for 4 h under argon protection until the reaction was finished. The resultant was filtered, dried by rotary evaporation and washed with methanol. The organic phase was filtered, concentrated and purified by Prep-HPLC to obtain the title compound 4 (30 mg, 0.04823 mmol) with a yield of 20%.

MS m/z (ESI):623.5[M+1]⁺

¹H NMR (400 MHz, Methanol-d₄) δ 8.39 (d, J=2.0 Hz, 1H), 8.25 (d, J=2.0 Hz, 1H), 8.17 (dd, J=7.9, 1.5 Hz, 1H), 7.71-7.65 (m, 1H), 7.52-7.45 (m, 1H), 7.40-7.31 (m, 2H), 7.11-7.06 (m, 1H), 4.12-3.95 (m, 2H), 3.80 (d, J=1.6 Hz, 2H), 3.14-2.75 (m, 9H), 2.50 (d, J=13.8 Hz, 6H), 2.14 (q, J=7.2 Hz, 2H), 2.04 (s, 3H).

### Example 5

### N-(5-(3-(1,5-dimethyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide

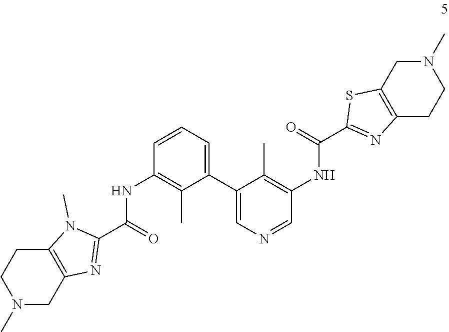 [SMILES: Cc1c(NC(=O)c2nc3c(s2)CN(C)CC3)cncc1-c1cccc(NC(=O)c2nc3c(n2C)CCN(C)C3)c1C]

A synthetic method similar to that of Example 3 was used to prepare the title product 5.

MS m/z (ESI): 571.4[M+1]⁺

¹H NMR (400 MHz, DMSO-d₆) δ 10.54 (s, 2H), 9.78 (s, 1H), 8.53 (s, 1H), 8.16 (s, 1H), 7.65 (dd, J=8.0, 1.2 Hz, 1H), 7.29 (t, J=7.8 Hz, 1H), 6.99 (dd, J=7.6, 1.4 Hz, 1H), 3.83 (s, 3H), 3.68 (t, J=1.6 Hz, 2H), 2.89 (t, J=5.8 Hz, 2H), 2.75 (t, J=5.8 Hz, 2H), 2.65 (t, J=3.6 Hz, 6H), 2.37 (d, J=7.9 Hz, 5H), 1.93 (d, J=9.1 Hz, 6H).

### Example 6

### N-(5-(3-(5-(2-hydroxypropyl)-1-methyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide

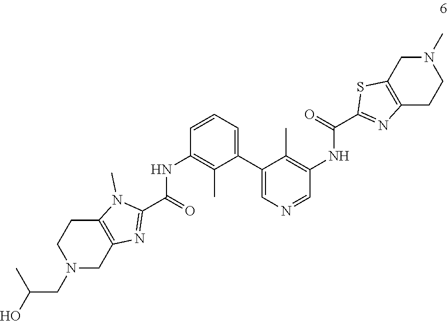 [SMILES: Cc1c(NC(=O)c2nc3c(s2)CN(C)CC3)cncc1-c1cccc(NC(=O)c2nc3c(n2C)CCN(CC(C)O)C3)c1C]

A synthetic method similar to that of Example 3 was used to prepare the title product 6.

MS m/z (ESI): 615.5[M+1]⁺

¹H NMR (400 MHz, DMSO-d₆) δ 10.57 (s, 1H), 9.78 (s, 1H), 8.56 (s, 1H), 8.19 (s, 1H), 7.73-7.67 (m, 1H), 7.32 (t, J=7.8 Hz, 1H), 7.01 (dd, J=7.6, 1.3 Hz, 1H), 4.37 (d, J=4.0 Hz, J H), 3.86 (s, 4H), 3.71 (d, J=1.8 Hz, 2H), 3.49 (s, 2H), 2.91 (d, J=6.0 Hz, 2H), 2.85-2.76 (m, 4H), 2.65 (s, 2H), 2.46 (d, J=6.9 Hz, 1H), 2.41 (s, 4H), 1.96 (d, J=8.6 Hz, 6H), 1.06 (d, J=6.1 Hz, 3H).

### Example 7

### (R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[5,4-b]pyridin-6-yl)methyl)pyrrolidine-3-carboxyl acid

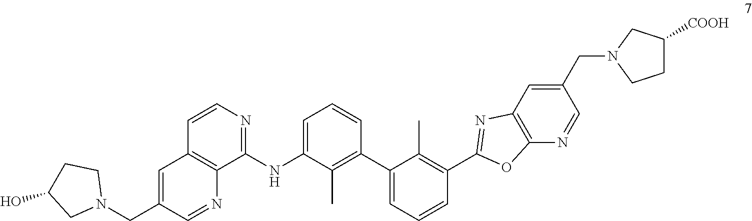 [SMILES: Cc1c(Nc2nccc3cc(CN4CC[C@@H](O)C4)cnc23)cccc1-c1cccc(-c2nc3cc(CN4CC[C@@H](C(=O)O)C4)cnc3o2)c1C]

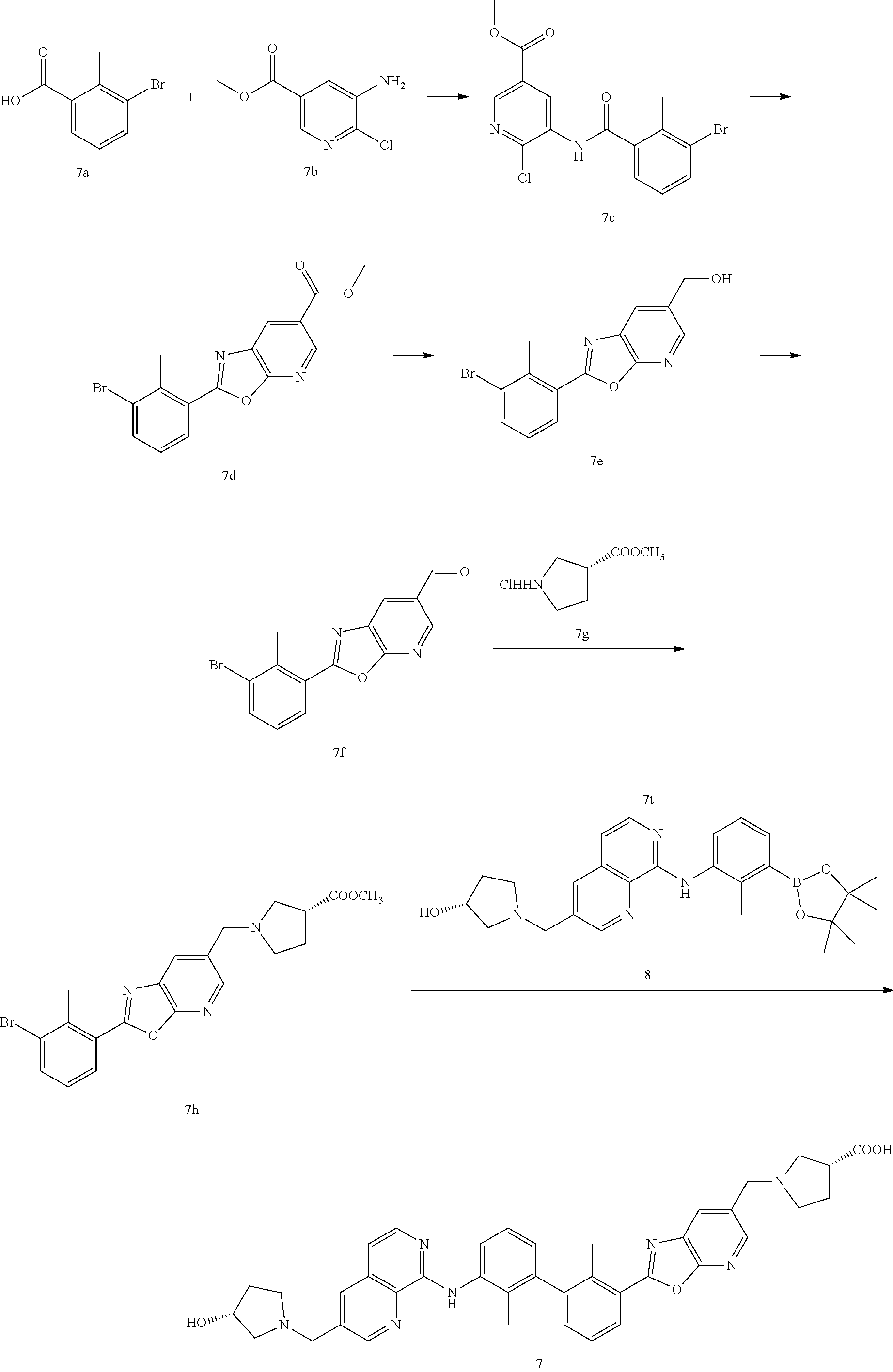

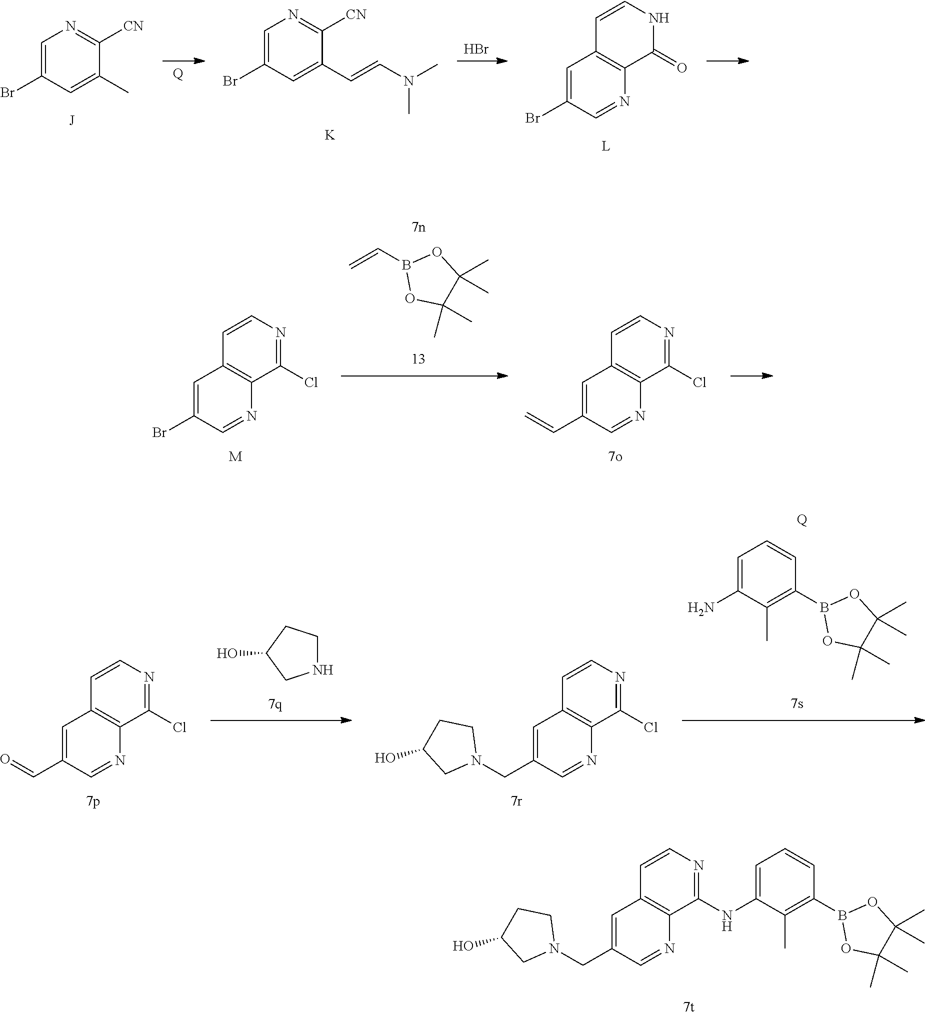

### Step 1 methyl 5-(3-bromo-2-methylbenzamido)-6-chloronicotinate 7c

Compound 7a (6.45 g, 30 mmol) and compound 7b (5.58 g, 30 mmol) were dissolved in pyridine (30 mL), and phosphorus oxychloride (9.18 g, 60 mmol) was slowly added dropwise in an ice bath, which were stirred at room temperature for 3 h until the reaction was finished. Ice water was added. After that, the resultant was extracted with ethyl acetate. The organic phase was washed with saturated copper sulfate solution and saturated brine, dried over anhydrous sodium sulfate, filtered, concentrated and purified by column chromatography to obtain the title compound 7c (4 g, 10.44 mmol) with a yield of 35%.

MS m/z (ESI): 385.1[M+1]⁺.

Step 2 methyl 2-(3-bromo-2-methylphenyl)oxazolo[5,4-b]pyridine-6-carboxylate 7d

Compound 7c (3 g, 7.832 mmol) and N,N′-dimethylethylenediamine (69 mg, 0.7832 mmol) were dissolved in toluene (50 mL), added with CuI (149 mg, 0.7832 mmol) and potassium carbonate (2.161 g, 15.664 mmol), heated to 120° C. under argon protection and stirred for 24 h until the reaction was finished. The reaction solution was added with water, extracted three times with ethyl acetate, washed with saturated brine, dried over anhydrous sodium sulfate, filtered, concentrated and purified by column chromatography to obtain the title compound 7d (1.5 g, 4.322 mmol) with a yield of 55%.

MS m/z (ESI): 347 [M+H]⁺.

### Step 3 (2-(3-bromo-2-methylphenyl)oxazolo[5,4-b]pyridin-6-yl)methanol 7e

Compound 7d (3 g, 8.645 mmol) was dissolved in dichloromethane (60 mL), cooled to −78° C. under argon protection. DIBAL-H (11.5 mL, 17.29 mmol) was then slowly added. The reaction solution was stirred at this temperature for 1 h, and then warmed to room temperature, and quenched by adding water. After that, saturated sodium potassium tartrate was added. The resultant was then extracted with ethyl acetate. The organic phase was washed with saturated sodium carbonate solution and brine, dried over anhydrous sodium sulfate, filtered, and concentrated to obtain the title compound 7e (2.2 g, 6.89 mmol) with a yield of 79%.

MS m/z (ESI) 319 [M+1]⁺.

### Step 4 2-(3-bromo-2-methylphenyl)oxazolo[5,4-b]pyridine-6-formaldehyde 7f

Compound 7e (1.5 g, 4.702 mmol) was dissolved in dichloromethane (50 mL), and then Dess Martin oxidant (3.987 g, 9.404 mmol) was added, which were reacted at room temperature for two hours until the reaction was finished. Sodium thiosulfate solution was added and stirred for 1 h. The resultant was extracted three times with ethyl acetate, washed with saturated brine, dried over anhydrous sodium sulfate, filtered, concentrated and purified by column chromatography to obtain the title compound 7f (1.2 g, 3.785 mmol) with a yield of 80%.

MS m/z (ESI): 317.1 [M+H]⁺.

### Step 5 methyl (R)-1-((2-(3-bromo-2-methylphenyl)oxazolo[5,4-b]pyridin-6-yl)methyl)pyrrolidine-3-carboxylate 7h

Compound 7f (1.2 g, 3.785 mmol) and methyl (R)-pyrrolidine-3-carboxylate hydrochloride 7g (1.249 g, 7.57 mmol) were dissolved in dichloromethane (60 mL), stirred for 1 h and added with sodium triacetoxyborohydride (2.407 g, 11.356 mmol), which were stirred overnight at room temperature until the reaction was finished. The reaction solution was added with water and ethyl acetate for extraction. The organic phase was washed with saturated brine, dried over anhydrous sodium sulfate, filtered, concentrated and purified by column chromatography to obtain the title compound 7h (0.7 g, 1.627 mmol) with a yield of 43%.

MS m/z (ESI): 430.2[M+1]⁺.

### Step 6 (E)-5-bromo-3-(2-(dimethylamino)vinyl)-2-cyanopyridine 7k

Compound 7j (5-bromo-3-methyl-2-cyanopyridine) (30 g, 152 mmol) and compound N,N-dimethylformamide dimethylacetal (36 g, 304 mmol) were dissolved in N,N-dimethylformamide (100 mL), heated to 140° C. and reacted for 20 h. The solvent was then dried by rotary evaporation. Ethyl acetate was added to the sample mixture for purification by column chromatography to obtain the title compound 7k (18 g, 91.3 mmol) with a yield of 60%.

### Step 7 3-bromo-1,7-naphthyridin-8-(7H)-one 71

Compound (E)-5-bromo-3-(2-(dimethylamino)vinyl)-2-cyanopyridine 7k (18 g, 91.3 mmol) was dissolved in ethanol (80 mL), and hydrobromic acid (23 g, 285 mmol) was added, which were heated to 90° C., reacted for 6 h, and filtered to obtain a yellow solid. Hydrobromic acid was washed off with sodium bicarbonate solution and the title compound 71 (6.8 g, 30.2 mmol) was obtained with a yield of 33.1%.

MS m/z (ESI): 227[M+1]⁺.

### Step 8 3-bromo-8-chloro-1,7-naphthyridine 7m

Compound 3-bromo-1,7-naphthyridin-8-(7H)-one 71 (6.8 g, 30.2 mmol) was dissolved in phosphorus oxychloride (50 mL), heated to 110° C. and reacted for 3 h. The reaction solution was dropped into ice water, extracted three times with ethyl acetate. The organic phase was dried over anhydrous sodium sulfate, and purified by column chromatography to obtain the title compound 7m (4.1 g, 16.8 mmol) with a yield of 55.6%.

MS m/z (ESI): 244.9[M+1]⁺.

### Step 9 8-chloro-3-vinyl-1,7-diazanaphthalene 7o

Compound 7m (2 g, 8.23 mmol) and compound 7n (1.394 g, 9.053 mmol) were dissolved in 1,4-dioxane/water (v/v=4/1) (50 mL), and [1,1′-bis(diphenylphosphino)ferrocene]palladium dichloride (601 mg, 0.823 mmol) and sodium carbonate (1.744 g, 16.46 mmol) were added, which were reacted at 110° C. for 4 h under argon protection until the reaction was finished. The reaction solution was added with water, extracted three times with ethyl acetate, washed with saturated brine, dried over anhydrous sodium sulfate, filtered, concentrated and purified by column chromatography to obtain the title compound 7o (800 mg, 4.21 mmol) with a yield of 51%.

MS m/z (ESI): 191[M+1]⁺.

### Step 10 8-chloro-1,7-diazanaphthalene-3-formaldehyde 7p

Potassium osmate (31 mg, 0.0842 mmol) was added to compound 7o (800 mg, 4.21 mmol) in 1,4-dioxane/water (v/v=4/1) (50 mL) in ice bath, and stirred for 1 h. After that, sodium periodate (1.8 g, 8.42 mmol) was added, and the mixture was heated to room temperature and stirred overnight until the reaction was finished. The reaction solution was added with water, extracted three times with ethyl acetate, washed with saturated brine, dried over anhydrous sodium sulfate, filtered, concentrated and purified by column chromatography to obtain the title compound 7p (600 mg, 3.125 mmol) with a yield of 74%.

MS m/z (ESI): 193[M+1]⁺.

### Step 11 (R)-1-((8-chloro-1,7-diazanaphthalen-3-yl)methyl)pyrrolidin-3-ol 7r

Compound 7p (800 mg, 4.1666 mmol) and (R)-pyrrolidin-3-ol 7q (1.0879 g, 12.513 mmol) were dissolved in dichloromethane (40 mL), added with acetic acid (250 mg, 0.41666 mmol), and stirred for 1 h. Sodium triacetoxyborohydride (2.65 g, 12.513 mmol) was then added, and the mixture was stirred at room temperature overnight until the reaction was finished. The reaction solution was added with saturated sodium bicarbonate solution and extracted with ethyl acetate. The organic phase was washed with saturated brine, dried over anhydrous sodium sulfate, filtered, concentrated and purified by column chromatography to obtain the title compound 7r (0.72 g, 2.737 mmol) with a yield of 65%.

MS m/z (ESI): 264.1[M+1]⁺.

### Step 12 (R)-1-((8-((2-methyl-3-(4,4,5,5-tetramethyl-1,3,2-dioxaborolan-2-yl)phenyl)amino)-1,7-diazanaphthalen-3-yl)methyl)pyrrolidin-3-ol 7t

Compound 7r (700 mg, 2.661 mmol) and compound 7s (682 mg, 2.927 mmol) were dissolved in isopropanol (40 mL), and HCl/dioxane (1.33 mL, 5.322 mmol) was added, which were heated to 95° C. and stirred for 4 h. The reaction solution was dried by rotary evaporation, added with saturated sodium bicarbonate solution and extracted with ethyl acetate. The organic phase was washed with saturated brine, dried over anhydrous sodium sulfate, filtered, concentrated and purified by column chromatography to obtain the title compound 7t (0.65 g, 1.41 mmol) with a yield of 53%.

MS m/z (ESI): 461.3[M+1]⁺.

### Step 13 (R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[5,4-b]pyridin-6-yl)methyl)pyrrolidine-3-carboxylic acid 7

Compound 7h (120 mg, 0.279 mmol) and compound 7t (192 mg, 0.4186 mol) were dissolved in tert-butanol/water (v/v=2/1) (15 mL), and 1,1′-bis(di-cyclohexylphosphine)ferrocene palladium dichloride (21 mg, 0.02791 mmol) and cesium carbonate (240 mg, 0.837 mmol) were added, which were reacted under argon protection at 100° C. for 4 h until the reaction was finished. The reaction solution was filtered, dried by rotary evaporation and washed with methanol. The organic phase was filtered, concentrated and purified by Prep-HPLC to obtain the title compound 7 (34 mg, 0.0508 mmol) with a yield of 18%.

MS m/z (ESI): 670.5[M+1]⁺.

¹H NMR (400 MHz, DMSO-d₆) δ 9.30 (s, 1H), 8.83 (d, J=2.1 Hz, 1H), 8.44 (dd, J=8.2, 1.3 Hz, 1H), 8.31 (d, J=1.9 Hz, 1H), 8.14 (ddd, J=14.7, 7.3, 1.7 Hz, 3H), 8.03 (d, J=5.8 Hz, 1H), 7.51 (t, J=7.7 Hz, J H), 7.40 (dd, J=7.6, 1.5 Hz, 1H), 7.32 (t, J=7.8 Hz, 1H), 7.15 (d, J=5.8 Hz, 1H), 6.89 (dd, J=7.5, 1.3 Hz, 1H), 4.19 (dt, J=6.8, 3.4 Hz, 1H), 3.75 (dq, J=21.2, 13.4 Hz, 5H), 3.00-2.85 (m, 2H), 2.70 (ddd, J=9.9, 7.2, 3.1 Hz, 2H), 2.62 (q, J=7.4, 6.7 Hz, 2H), 2.58-2.49 (m, 2H), 2.44 (s, 3H), 2.35 (dd, J=9.7, 3.7 Hz, 1H), 2.06 (s, 3H), 1.96 (dq, J=21.1, 6.9 Hz, 3H), 1.61-1.51 (m, 1H).

### Example 8

### (R)-1-((7-chloro-2-(2-methyl-3-(4-methyl-5-(5-methyl-4,5,6,7-tetrahydrothiazolo[5,4-c]pyridine-2-carboxamido)pyridin-3-yl)phenyl)benzo[d]oxazol-5-yl)methyl)pyrrolidine-3-carboxylic acid

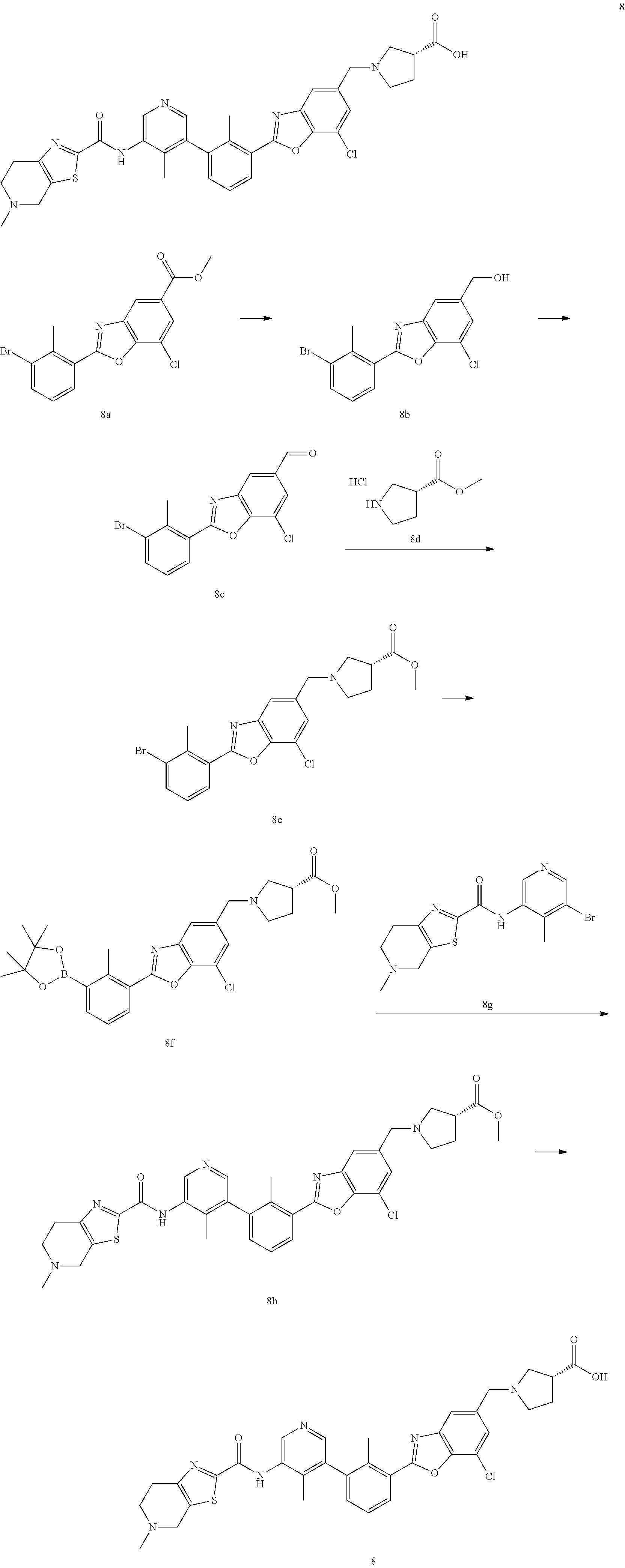

### Step 1 (2-(3-bromo-2-methylphenyl)-7-chlorobenzo[d]oxazol-5-yl)methanol

Compound 8a (1.90 g, 5.00 mmol) was dissolved in THF (20 mL). The atmosphere was replaced with nitrogen for three times at room temperature. DIBAL-H (10 mL, 10 mmol) was added at −78° C. The system was stirred at room temperature for 20 h. The reaction solution was added with water, extracted three times with DCM (30 mL×3), washed with saturated brine for several times, dried over anhydrous sodium sulfate, filtered, concentrated to obtain the target compound 8b (crude product, white solid), which was directly used in the next step without further purification.

MS m/z (ESI): 354[M+1]⁺.

### Step 2 2-(3-bromo-2-methylphenyl)-7-chlorobenzo[d]oxazole-5-formaldehyde

The crude compound 8b obtained from the last step was dissolved in DCM/1,4-dioxane (10/5 mL). DMP (10.6 g, 25 mmol) was added at room temperature. The reaction solution was stirred at room temperature for 20 h. The reaction solution was adjusted to neutral with saturated NaHCO₃, extracted three times with DCM (30 mL×3), washed with saturated brine for several times, dried over anhydrous sodium sulfate, filtered, concentrated and purified by column chromatography (SiO₂, PE/EA=5:1) to obtain the title compound 8c (780 mg) as crude product, which was used in the next reaction step without further purification.

MS m/z (ESI): 352[M+1]⁺.

### Step 3 methyl (R)-1-(((2-(3-bromo-2-methylphenyl)-7)-chlorobenzo[d]oxazol-5-yl)methyl)pyrrolidine-3-carboxylate

Sodium borohydride acetate (1.40 g, 6.66 mmol) was added to a mixture of compound 8c (780 mg, 2.22 mmol) and compound 8d (431 mg, 3.34 mmol) in DCM (10 mL), and stirred at room temperature for 20 h. The reaction solution was adjusted to neutral with saturated NaHCO₃ aqueous solution, extracted three times with dichloromethane, dried over anhydrous sodium sulfate, filtered, concentrated and purified by column chromatography (SiO₂, PE/EA=20:1 to 2:1) to obtain the target compound 8e (463 mg, white solid), which was used directly in the next step without further purification.

MS m/z (ESI): 463[M+1]⁺.

### Step 4 methyl (R)-1-(((7-chloro-2-(2-methyl-3-(4,4,5,5-tetramethyl-1,3,2-dioxaboran-2-yl)phenyl)benzo[d]oxazol-5-yl)methyl)pyrrolidine-3-carboxylate

Compound 8e (463 mg, 1.00 mmol), BP (506 mg, 2.00 mmol), KOAc (300 mg, 3.00 mmol), Pd(dppf)Cl₂ (463 mg, 1.00 mmol) and 1,4-dioxane (20 mL) were successively introduced into a single-necked bottle. The atmosphere was replaced with N₂ for three times at room temperature. The reaction was carried out at 110° C. with stirring for 5 h. The reaction system was concentrated, and then purified by column chromatography (SiO₂, PE/EA=5:1 to 1:1) to obtain the target compound 8f (312 mg, yield 77%) as a white solid.

MS m/z (ESI): 511[M+1]⁺.

### Step 5 methyl (R)-1-(((7-chloro-2-(2-methyl-3-(4-methyl-5-(5-methyl-4,5,6,7-tetrahydrothiazolo[5,4-c]pyridine-2-carboxamido)pyridin-3-yl)phenyl)benzo[d]oxazol-5-yl)methyl)pyrrolidine-3-carboxylate

Compound 8g (346 mg, 0.94 mmol) and compound 8f (401 mg, 0.78 mmol), 1,4-dioxane/H₂O (4:1, 25 mL), K₂CO₃ (322 mg, 2.34 mmol) and Pd(PPh₃)₄ (180 mg, 0.15 mmol) were successively introduced into a single-necked bottle. The atmosphere was replaced with N₂ for three times at room temperature. The reaction was carried out at 110° C. for 3 h. The reaction solution was added with water, and extracted with EtOAc (50 mL×3). The organic phases were combined and dried over anhydrous sodium sulfate, filtered, concentrated and purified by column chromatography (SiO₂, DCM/MeOH=20:1) to obtain the target compound 8h (190 mg, not pure) as a brown oil, which was used directly in the next step without further purification. MS m/z (ESI): 671[M+1]⁺.

### Step 6 (R)-1-((7-chloro-2-(2-methyl-3-(4-methyl-5-(5-methyl-4,5,6,7-tetrahydrothiazolo[5,4-c]pyridine-2-carboxamido)pyridin-3-yl)phenyl)benzo[d]oxazol-5-yl)methyl)pyrrolidine-3-carboxylic acid

Compound 8h (190 mg, 0.28 mmol) was dissolved in methanol (5.0 mL), and added with LiOH aqueous solution (1 M, 1.4 mL). The reaction was stirred at 50° C. for 3 h until the reaction was finished. The pH of the reaction system was adjusted to weakly acidic with acetic acid, which was concentrated and then purified by column chromatography (C18, MeCN/H₂O) to obtain the title compound 8 (10.0 mg, white solid).

MS m/z (ESI): 657[M+1]⁺

¹H NMR (400 MHz, DMSO-d₆) δ 10.76 (s, 11H), 8.59 (s, 11-H), 8.28 (s, 1H), 8.18 (d, J=7.8 Hz, 1H), 8.00 (s, 1H), 7.76 (s, 1H), 7.57 (d, J=7.7 Hz, 1H), 7.46 (d, J=7.4 Hz, 1H), 4.51 (s, 2H), 3.65 (s, 8H), 3.19 (d, J=6.2 Hz, 3H), 2.98 (s, 3H), 2.40 (s, 2H), 2.30-2.10 (m, 2H), 1.98 (s, 3H), 1.74-1.64 (m, 1H).

### Example 9

### (R)-1-(((7-chloro-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid

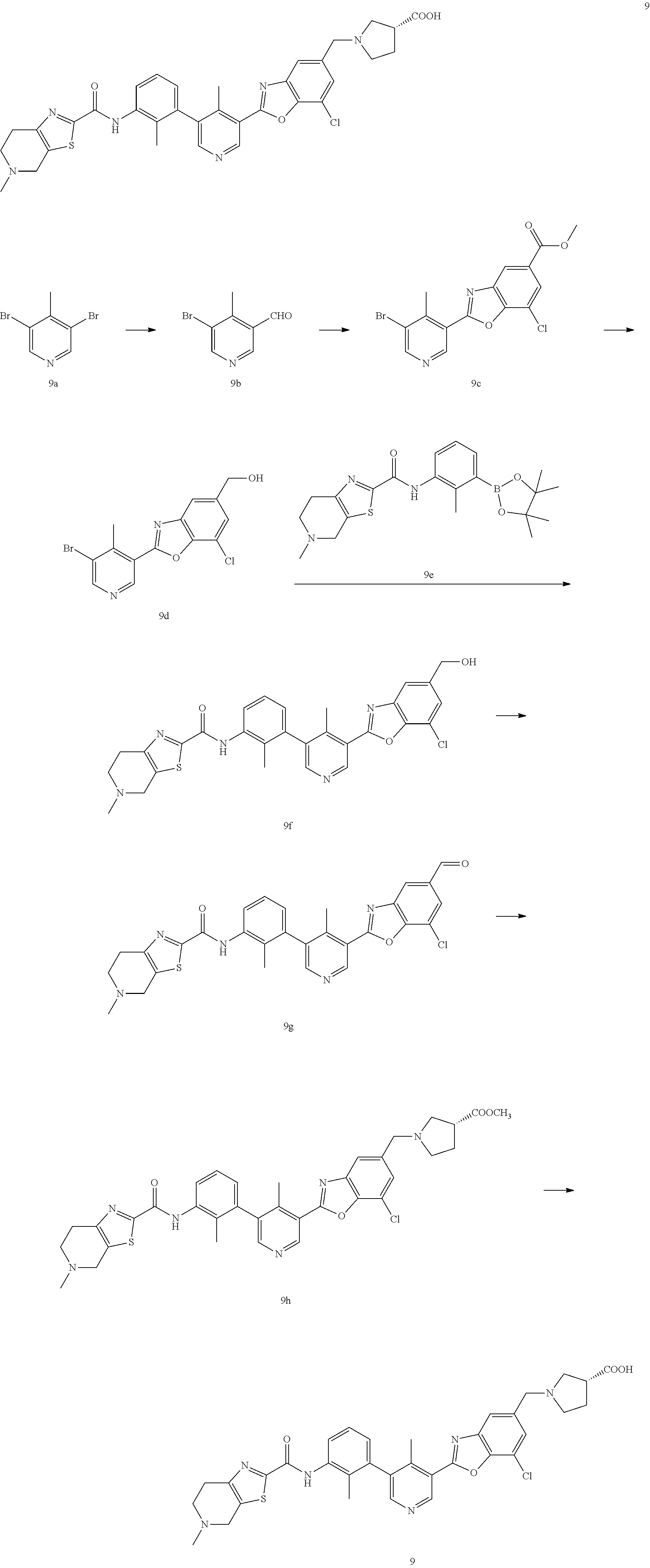

### Step 1 5-bromo-4-methylnicotine 9b

Compound 9a (20 g, 79.7 mmol) was dissolved in dry THF (500 mL), and isopropylmagnesium bromide (87.7 mmol, 1M/THF) was slowly added dropwise under an ice-salt bath condition. After the addition was completed, stirring was continued for 2 h, and then, DMF (11.6 g, 159.4 mmol) was added at one time followed by continued stirring for 1 h. The reaction was then quenched by adding water dropwise, and extracted with ethyl acetate. The organic phases were combined and washed once with saturated brine, dried over anhydrous sodium sulfate, filtered, concentrated and purified by column chromatography (PE:EA=1:5) to obtain the title compound 9b (11 g, 55 mmol) with a yield of 69%.

MS m/z (ESI): 202[M+1]⁺.

### Step 2 methyl 2-(5-bromo-4-methylpyridin-3-yl)-7-chlorobenzo[d]oxazole-5-carboxylate 9c

Compound 9b (10 g, 50 mmol) and compound methyl 3-amino-5-chloro-4-hydroxybenzoate (10 g, 50 mmol) were dissolved in a mixed solvent of EA/EtOH (100 mL/30 mL) and heated to 70° C. with stirring for 2h. After cooling to room temperature, the solvent was concentrated and removed. DCM (150 mL) was added for dissolving the residue. DDQ (12.5 g, 55 mmol) was added under stirring. After stirring for 1 h, DCM (100 mL) was added to the reaction solution for dilution. The reaction was quenched by adding sodium thiosulfate aqueous solution and sodium bicarbonate aqueous solution. The layers were separated, and then the aqueous phase was extracted with DCM. The organic phases were combined, dried over anhydrous sodium sulfate, filtered, concentrated and purified by column chromatography (DCM:EA=2:1) to obtain the title compound 9c (6.9 g, 18 mmol) with a yield of 36%.

MS m/z (ESI): 383[M+1]⁺.

### Step 3 (2-(5-bromo-4-methylpyridin-3-yl)-7-chlorobenzo[d]oxazol-5-yl)methanol 9d

Compound 9c (10 g, 26 mmol) was dissolved in dry THF (500 mL), cooled to −78° C. in a dry ice acetone bath, and LiAH₄ (105 mmol, 1M/THF) was added slowly dropwise. After the addition was complete, and the temperature was slowly raised to −40° C. with stirring was continued for 3 h. The end of the reaction was determined by TLC detection. The resultant was cooled to −78° C., slowly added with water to quench the reaction, and extracted with ethyl acetate. The combined organic phases were washed once with saturated brine, dried over anhydrous sodium sulfate, filtered, concentrated, and then subjected to column chromatography purification (DCM:MeOH=15:1) to obtain the title compound 9d (5.6 g, 15.8 mmol) with a yield of 60.7%.

MS m/z (ESI): 355[M+1]⁺.

### Step 4 N-(3-(5-(7-chloro-5-(hydroxymethyl)benzo[d]oxazol-2-yl]-4-methylpyridin-3-yl)-2-methylphenyl)-5-methyl-4,5,6,7-tetrahydrothiazolo[5,4-c]pyridine-2-carboxamide 9f

Compound 9d (854 mg, 2.42 mmol), compound 9e (1 g, 2.42 mmol), Pd(dppf)Cl₂ (177 mg, 0.242 mmol) and K₂CO₃ (1 g, 7.26 mmol) were dissolved in dioxane (30 mL) and H₂O (6 mL), heated to 90° C. under argon protection and stirred for 3 h. After cooling to room temperature, the layers were separated. The aqueous phase was extracted with ethyl acetate. The organic phases were combined, washed once with saturated brine, dried over anhydrous sodium sulfate, filtered, concentrated and purified by column chromatography to obtain the title compound 9f (1 g, 1.79 mmol) with a yield of 73.8%.

MS m/z (ESI): 560.3[M+1]⁺.

### Step 5 N-(3-(5-(7-chloro-5-formylbenzo[d]oxazol-2-yl)-4-methylpyridin-3-yl)-2-methylphenyl)-5-methyl-4,5,6,7-tetrahydrothiazolo[5,4-c]pyridine-2-carboxamide 9g

Compound 9f (240 mg, 0.429 mmol) was dissolved in DCM (20 mL), and added with DMP (273 mg, 0.643 mmol) at room temperature, and stirring was continued for 1 h. The reaction was quenched by adding sodium thiosulfate aqueous solution and sodium bicarbonate aqueous solution. The layers were separated. The aqueous phase was extracted with DCM. The organic phases were combined, dried over anhydrous sodium sulfate, filtered, concentrated and purified by column chromatography to obtain the title compound 9g (200 mg, 0.358 mmol) with a yield of 83.7%.

MS m/z (ESI): 558.2[M+1]⁺.

### Step 6 methyl (R)-1-(((7-chloro-2-(4-methyl-5-(2-methyl-3-(5-methyl-4,5,6,7-tetrahydrothiazole)[5,4-c]pyridine-2-carboxamido)phenyl)pyridin-3-yl)benzo[d]oxazol-5-yl)methyl)pyrrolidine-3-carboxylate 9h

Compound 9g (200 mg, 0.358 mmol) and methyl (R)-pyrrolidine-3-carboxylate hydrochloride (92.6 mg, 0.559 mmol) were dissolved in dichloromethane (20 mL). The reaction was stirred at room temperature for 30 minutes. After that, sodium borohydride acetate (379 mg, 1.79 mmol) was added, and stirred at room temperature overnight until the reaction was finished. The reaction solution was quenched with water, and extracted three times with dichloromethane. The organic phases were combined and washed with saturated brine, dried over anhydrous sodium sulfate, filtered, concentrated and purified by column chromatography to obtain the title compound 24h (100 mg, 0.149 mmol) as a white solid with a yield of 41.6%.

MS m/z (ESI): 671.4[M+1]⁺.

### Step 7 (R)-1-(((7-chloro-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid 9

Compound 24h (100 mg, 0.149 mmol) was dissolved in a mixed solvent of acetonitrile/H₂O (5 mL/5 mL), added with LiOH (7.1 mg, 0.298 mmol), and stirred at room temperature for 2 h. The end of the reaction was determined by LCMS detection. The pH value was adjusted to weakly acidic with acetic acid. The resultant was purified by pre-HPLC to obtain the title compound 9 (40 mg, 0.061 mmol) as a white solid with a yield of 40.9%.

MS m/z (ESI): 657.4[M+1]⁺

¹H NMR (400 MHz, Methanol-d₄) δ 9.25 (s, 1H), 8.47 (s, 1H), 7.88 (s, 1H), 7.73 (d, J=8.0 Hz, 1H), 7.66 (d, J=1.4 Hz, 1H), 7.39 (t, J=7.8 Hz, 1H), 7.15 (dd, J=7.6, 1.3 Hz, 1H), 4.37 (d, J=13.0 Hz, 1H), 4.26 (d, J=13.1 Hz, 1H), 3.81 (d, J=1.6 Hz, 2H), 3.42-3.33 (m, 1H), 3.24 (m, 2H), 3.18-3.03 (m, 2H), 3.00 (t, J=5.7 Hz, 2H), 2.91 (t, J=5.8 Hz, 2H), 2.57 (s, 3H), 2.52 (s, 3H), 2.26 (m, 2H), 2.06 (d, J=3.4 Hz, 3H).

### Example 10

### (R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridin-6-yl)methyl)pyrrolidine-3-carboxylic acid

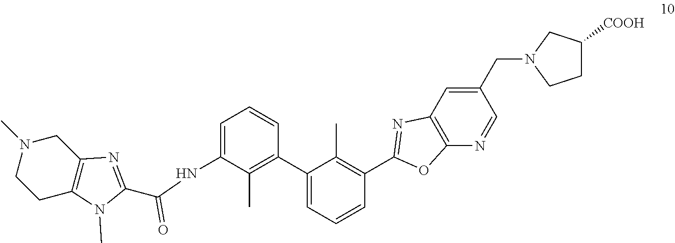 [SMILES: Cc1c(NC(=O)c2nc3c(n2C)CCN(C)C3)cccc1-c1cccc(-c2nc3cc(CN4CC[C@@H](C(=O)O)C4)cnc3o2)c1C]

A synthetic method similar to that of Example 4 was used to prepare the title product 10.

MS m/z (ESI): 620.4[M+1]⁺

¹H NMR (400 MHz, DMSO-d₆) δ 10.66 (s, 1H), 9.93 (s, 1H), 8.53 (d, J=2.0 Hz, 1H), 8.46 (d, J=2.0 Hz, 1H), 8.14 (dd, J=8.0, 1.4 Hz, 1H), 7.58 (dd, J=8.0, 1.3 Hz, 1H), 7.51 (d, J=7.8 Hz, 1H), 7.38 (dd, J=7.6, 1.5 Hz, 1H), 7.30 (s, 1H), 7.02 (dd, J=7.7, 1.3 Hz, 1H), 4.60 (s, 2H), 4.30 (d, J=85.3 Hz, 5H), 3.89 (s, 3H), 3.85-3.56 (m, 3H), 3.24 (s, 2H), 2.97 (d, J=15.1 Hz, 5H), 2.41 (s, 3H), 2.21 (s, 1H), 1.93 (s, 3H).

### Example 11

### (R)-1-(((7-chloro-2-(5-(3-((3-((((R)-3-hydroxypyrrolidin-1-yl)methyl)-1,7-naphthyridin-8-yl)amino)-2-methylphenyl)-4-methylpyridin-3-yl)benzo[d]oxazol-5-yl)methyl)pyrrolidine-3-carboxylic acid trifluoroacetate

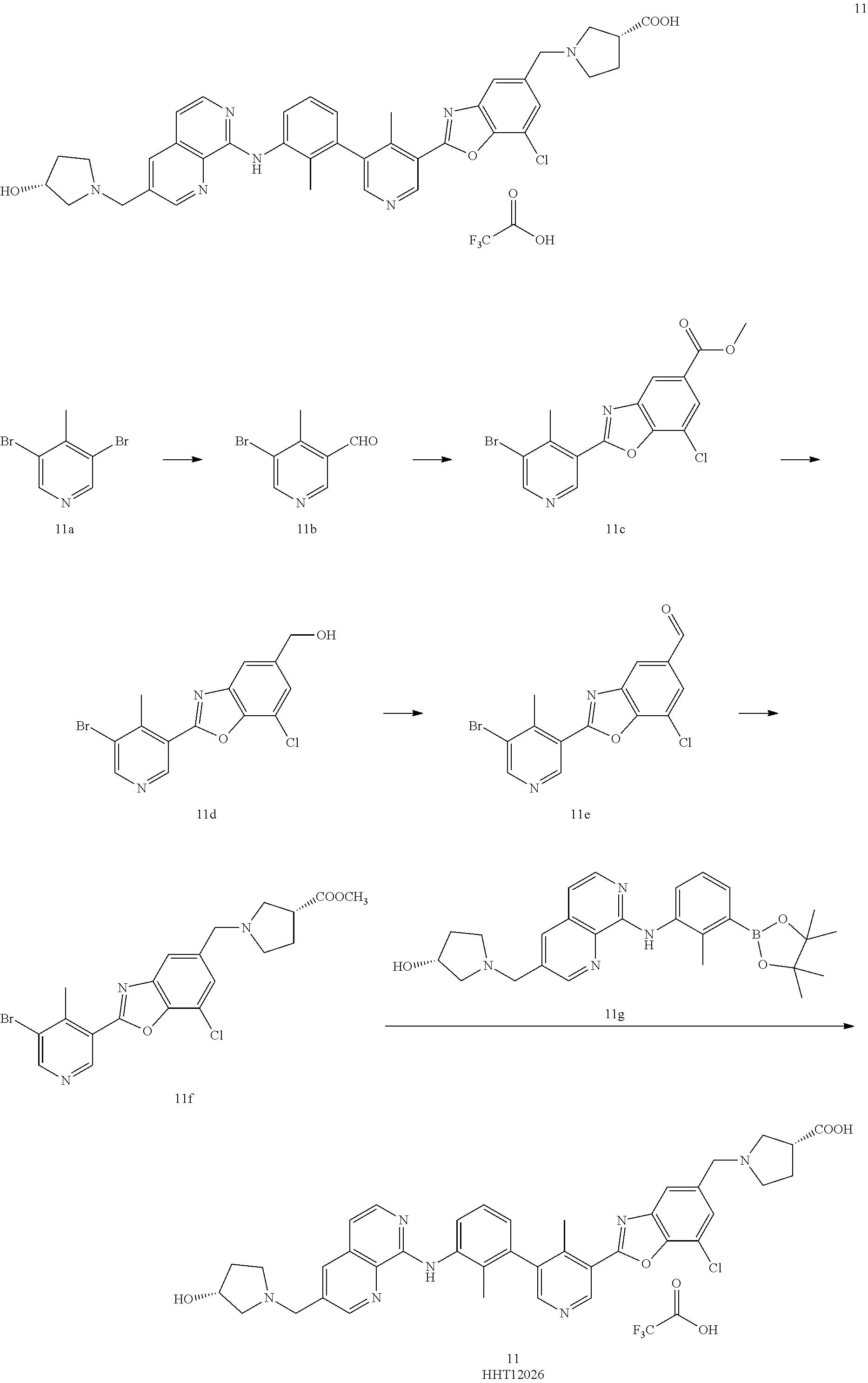

### Step 1 5-bromo-4-methylnicotine 11b

Compound 11a (20 g, 79.7 mmol) was dissolved in dry THF (500 mL), and isopropylmagnesium bromide (87.7 mmol, 1M/THF) was slowly added dropwise under ice-salt bath condition. After the addition was completed, stirring was continued for 2 h, and then, DMF (11.6 g, 159.4 mmol) was added at one time with continued stirring for 1 h. The reaction was then quenched by adding water dropwise. The resultant was extracted with ethyl acetate. The organic phases were combined, washed once with saturated brine, dried over anhydrous sodium sulfate, filtered, concentrated and purified by column chromatography (PE:EA=1:5) to obtain the title compound 11b (11 g, 55 mmol) with a yield of 69%.

MS m/z (ESI): 202[M+1]⁺.

### Step 2 methyl 2-(5-bromo-4-methylpyridin-3-yl)-7-chlorobenzo[d]oxazole-5-carboxylate 11c

Compound 11b (10 g, 50 mmol) and compound methyl 3-amino-5-chloro-4-hydroxybenzoate (10 g, 50 mmol) were dissolved in a mixed solvent of EA/EtOH (100 mL/30 mL) and heated to 70° C. with stirring for 2 h. After cooling to room temperature, the reaction solution was concentrated to remove solvent. The resultant was dissolved by adding DCM (150 mL), added with DDQ (12.5 g, 55 mmol) and stirred for 1 h, DCM (100 mL) was added to the reaction solution for dilution. The reaction was quenched by adding sodium thiosulfate aqueous solution and sodium bicarbonate aqueous solution. The layers were separated, and then the aqueous phase was extracted with DCM. The organic phases were combined, dried over anhydrous sodium sulfate, filtered, concentrated and purified by column chromatography (DCM:EA=2:1) to obtain the title compound 11c (6.9 g, 18 mmol) with a yield of 36%.

MS m/z (ESI): 383[M+1]⁺.

### Step 3 (2-(5-bromo-4-methylpyridin-3-yl)-7-chlorobenzo[d]oxazol-5-yl)methanol 11d

Compound 11c (10 g, 26 mmol) was dissolved in dry THF (500 mL), cooled to −78° C. in a dry ice acetone bath, and LiAH₄ (105 mmol, 1M/THF) was slowly added dropwise. After the addition was complete, the temperature was slowly raised to −40° C. with continued stirring for 3 h. The end of the reaction was determined by TLC detection. The resultant was cooled to −78° C., slowly added with water to quench the reaction, and extracted with ethyl acetate. The organic phases were combined, washed once with saturated brine, dried over anhydrous sodium sulfate, filtered, concentrated, and then subjected to column chromatography purification (DCM:MeOH=15:1) to obtain the title compound 11d (5.6 g, 15.8 mmol) with a yield of 60.7%.

MS m/z (ESI): 355[M+1]⁺.

### Step 4 2-(5-bromo-4-methylpyridin-3-yl)-7-chlorobenzo[d]oxazole-5-formaldehyde 11e

Compound 26d (1 g, 2.83 mmol) was dissolved in DCM (20 mL), and added with DMP (1.8 g, 4.24 mmol) at room temperature, with continued stirring for 1 h. The reaction was quenched by adding sodium thiosulfate aqueous solution and sodium bicarbonate aqueous solution. The layers were separated. The aqueous phase was extracted with DCM. The organic phases were combined, dried over anhydrous sodium sulfate, filtered, concentrated and purified by column chromatography to obtain the title compound 11e (900 mg, 2.56 mmol) with a yield of 90.4%.

MS m/z (ESI): 353[M+1]⁺.

### Step 5 methyl (R)-1-(((2-(5-bromo-4-methylpyridin-3-yl)-7-chlorobenzo[d]oxazol-5-yl)methyl)pyrrolidine-3-carboxylate 11f

Compound 11e (200 mg, 0.569 mmol) and methyl (R)-pyrrolidine-3-carboxylate hydrochloride (188 mg, 1.138 mmol) were dissolved in dichloromethane (20 mL). The reaction was stirred at room temperature for 30 minutes. Then, sodium borohydride acetate (603 mg, 2.845 mmol) was added. The reaction was stirred at room temperature overnight until the reaction was finished. The reaction solution was quenched with water, and extracted three times with dichloromethane. The organic phases were combined, washed with saturated brine, dried over anhydrous sodium sulfate, filtered, concentrated and purified by column chromatography to obtain the title compound 11f (150 mg, 0.323 mmol) as a white solid with a yield of 56.7%.

MS m/z (ESI): 466.1[M+1]⁺.

### Step 6 (R)-1-((7-chloro-2-(5-(3-((3-(((R)-3-hydroxypyrrolidin-1-yl)methyl)-1,7-diazanaphthalen-8-yl)amino)-2-methylphenyl)-4-methylpyridin-3-yl)benzo[d]oxazol-5-yl)methyl)pyrrolidine-3-carboxylic acid 11

Compound 11f (150 mg, 0.323 mmol), compound 11g (148 mg, 0.323 mmol), Pd(dcydppf)Cl₂ (24.4 mg, 0.0323 mmol) and K₂CO₃ (134 mg, 0.969 mmol) were dissolved in dioxane (15 mL) and H₂O (3 mL), heated to 90° C. under argon protection and stirred for 3 h. After cooling to room temperature, the layers were separated. The aqueous phase was extracted with ethyl acetate. The organic phases were combined, washed once with saturated brine, dried over anhydrous sodium sulfate, filtered, concentrated and purified by column chromatography to obtain the title compound 11 (50 mg, 0.071 mmol) as a white solid with a yield of 22%.

MS m/z (ESI):704.4[M+1]⁺

¹H NMR (400 MHz, DMSO-d₆) δ 10.77-10.04 (m, 4H), 9.45-9.02 (m, 2H), 8.75-8.35 (m, 2H), 8.16-7.96 (m, 2H), 7.94 (d, J=6.2 Hz, 1H), 7.83-7.73 (m, 1H), 7.46 (t, J=7.8 Hz, 1H), 7.20 (dd, J=27.7, 6.9 Hz, 2H), 4.82-4.48 (m, 5H), 3.59-3.29 (m, 9H), 2.50 (s, 3H), 2.35-2.25 (m, 3H), 2.03 (s, 3H), 1.90-1.80 (m, 1H).

### Example 12

### (R)-1-(((7-chloro-2-(5-(3-(5-((S)-2-hydroxypropyl))-1-methyl-4,5,6,7-tetrahydro-1H-imidazo[4,5-c]pyridine-2-carboxamide)-2-methylphenyl)-4-methylpyridin-3-yl)benzo[d]oxazol-5-yl) methyl)pyrrolidine-3-carboxylic acid

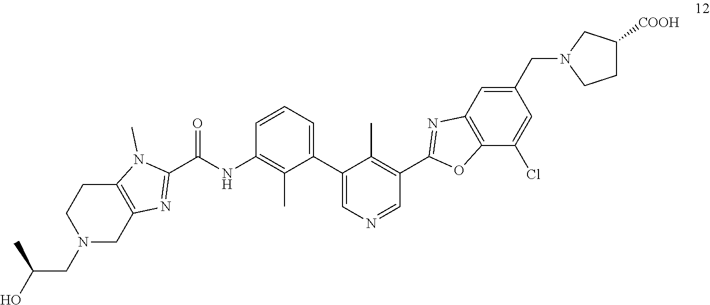 [SMILES: Cc1c(NC(=O)c2nc3c(n2C)CCN(C[C@H](C)O)C3)cccc1-c1cncc(-c2nc3cc(CN4CC[C@@H](C(=O)O)C4)cc(Cl)c3o2)c1C]

A synthetic method similar to that of Example 9 was used to prepare the title product 12.

MS m/z (ESI):698.4[M+1]⁺

¹H NMR (400 MHz, DMSO-d₆) δ 10.4-10.25 (m, 1H), 10.15-10.0 (m, 1H), 9.94 (s, 1H), 9.23 (s, 1H), 8.51 (s, 1H), 8.03 (d, J=1.5 Hz, 1H), 7.79 (d, J=1.4 Hz, 1H), 7.68-7.60 (m, 1H), 7.35 (t, J=7.8 Hz, 1H), 7.23 (s, 0.5H), 7.13-7.07 (m, 1H), 6.97 (s, 0.5H), 4.60-4.35 (m, 4H), 4.30-4.0 (m, 4H), 3.89 (s, 3H), 3.37-2.89 (m, 8H), 2.45 (s, 3H), 2.45-2.11 (m, 2H), 1.95 (s, 3H), 1.11 (d, J=6.1 Hz, 3H).

### Example 13

### (R)-1-(((7-chloro-2-(5-(3-(1,5-dimethyl-4,5,6,7-tetrahydro-1H-imidazo[4,5-c]pyridine-2-carboxamido)-2-methylphenyl)-4-methylpyridin-3-yl)benzo[d]oxazol-5-yl)methyl)pyrrolidine-3-carboxylic acid

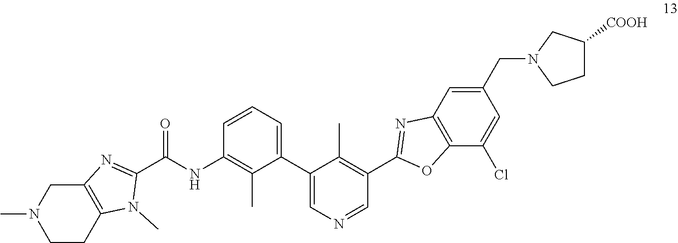 [SMILES: Cc1c(NC(=O)c2nc3c(n2C)CCN(C)C3)cccc1-c1cncc(-c2nc3cc(CN4CC[C@@H](C(=O)O)C4)cc(Cl)c3o2)c1C]

A synthetic method similar to that of Example 9 was used to prepare the title product 13.

MS m/z (ESI):654.4[M+1]⁺

¹H NMR (400 MHz, Methanol-d₄) δ 9.33 (s, 1H), 8.53 (s, 1H), 7.97 (d, J=1.5 Hz, 1H), 7.79 (dd, J=8.1, 1.3 Hz, 1H), 7.73 (d, J=1.5 Hz, 1H), 7.40 (t, J=7.8 Hz, 1H), 7.13 (dd, J=7.7, 1.4 Hz, 1H), 4.57 (s, 2H), 4.50-4.22 (m, 2H), 3.98 (s, 3H), 3.86-3.36 (m, 8H), 3.10 (m, 5H), 2.62 (s, 3H), 2.51-2.25 (m, 2H), 2.06 (s, 3H).

### Example 14

### (R)-1-((2-(3′-(5-((S)-2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridine-6-yl)methyl)pyrrolidine-3-carboxylic acid

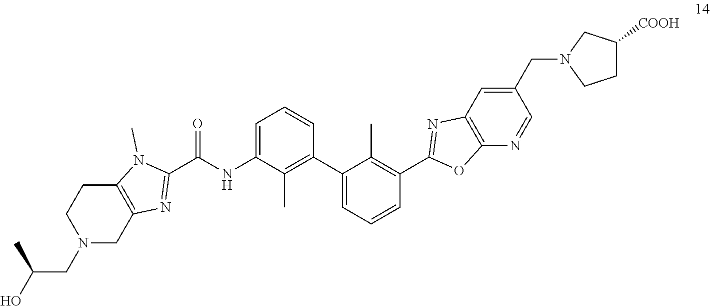 [SMILES: Cc1c(NC(=O)c2nc3c(n2C)CCN(C[C@H](C)O)C3)cccc1-c1cccc(-c2nc3cc(CN4CC[C@@H](C(=O)O)C4)cnc3o2)c1C]

A synthetic method similar to that of Example 4 was used to prepare the title product 14.

MS m/z (ESI): 664.5[M+1]⁺

¹H NMR (400 MHz, DMSO-d₆) δ 10.74 (d, J=77.3 Hz, 2H), 10.18 (s, 1H), 9.90 (s, 1H), 8.50 (dd, J=29.5, 2.0 Hz, 2H), 8.14 (dd, J=7.9, 1.4 Hz, 1H), 7.60 (d, J=8.0 Hz, 1H), 7.52 (t, J=7.7 Hz, 1H), 7.38 (dd, J=7.7, 1.5 Hz, 1H), 7.30 (t, J=7.8 Hz, 1H), 7.02 (dd, J=7.6, 1.4 Hz, 1H), 4.60 (s, 2H), 4.29 (s, 2H), 4.16 (ddd, J=9.3, 6.4, 2.6 Hz, 1H), 3.89 (s, 3H), 3.56 (d, J=60.2 Hz, 5H), 3.37-2.90 (m, 6H), 2.41 (s, 3H), 2.19 (d, J=16.6 Hz, 2H), 1.93 (s, 3H), 1.11 (d, J=6.1 Hz, 3H).

### Example 15

### N-(3-chloro-2-(3-(5-(2-hydroxypropyl)-1-methyl-4,5,6,7-tetrahydro-1H-imidazo[4,5-c]pyridine-2-carboxamido))-2-methylphenyl)pyridin-4-yl)-1,5-dimethyl-4,5,6,7-tetrahydro-1H-imidazo[4,5-c]pyridine-2-carboxamide 15

### Lithium 1,5-dimethyl-4,5,6,7-tetrahydro-1H-imidazo[4,5-c]pyridine-2-carboxylate 2-8

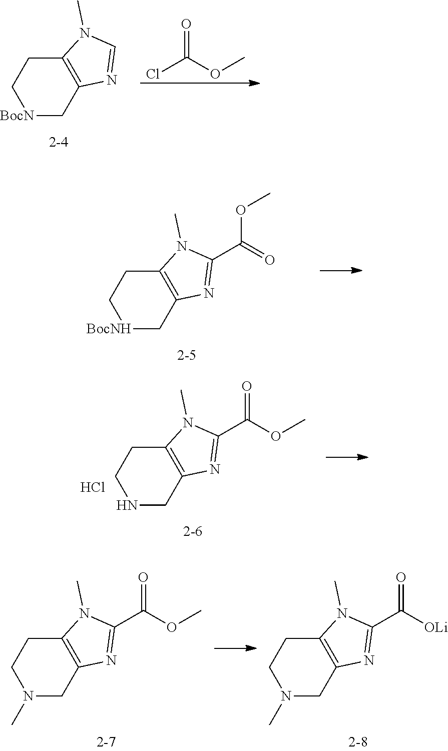

### Methyl 5-(2-(((tert-butyldimethylsilyl)oxy)propyl)-1-methyl-4,5,6,7-tetrahydro-1H-imidazo[4,5-c]pyridine-2-carboxylate 2-11

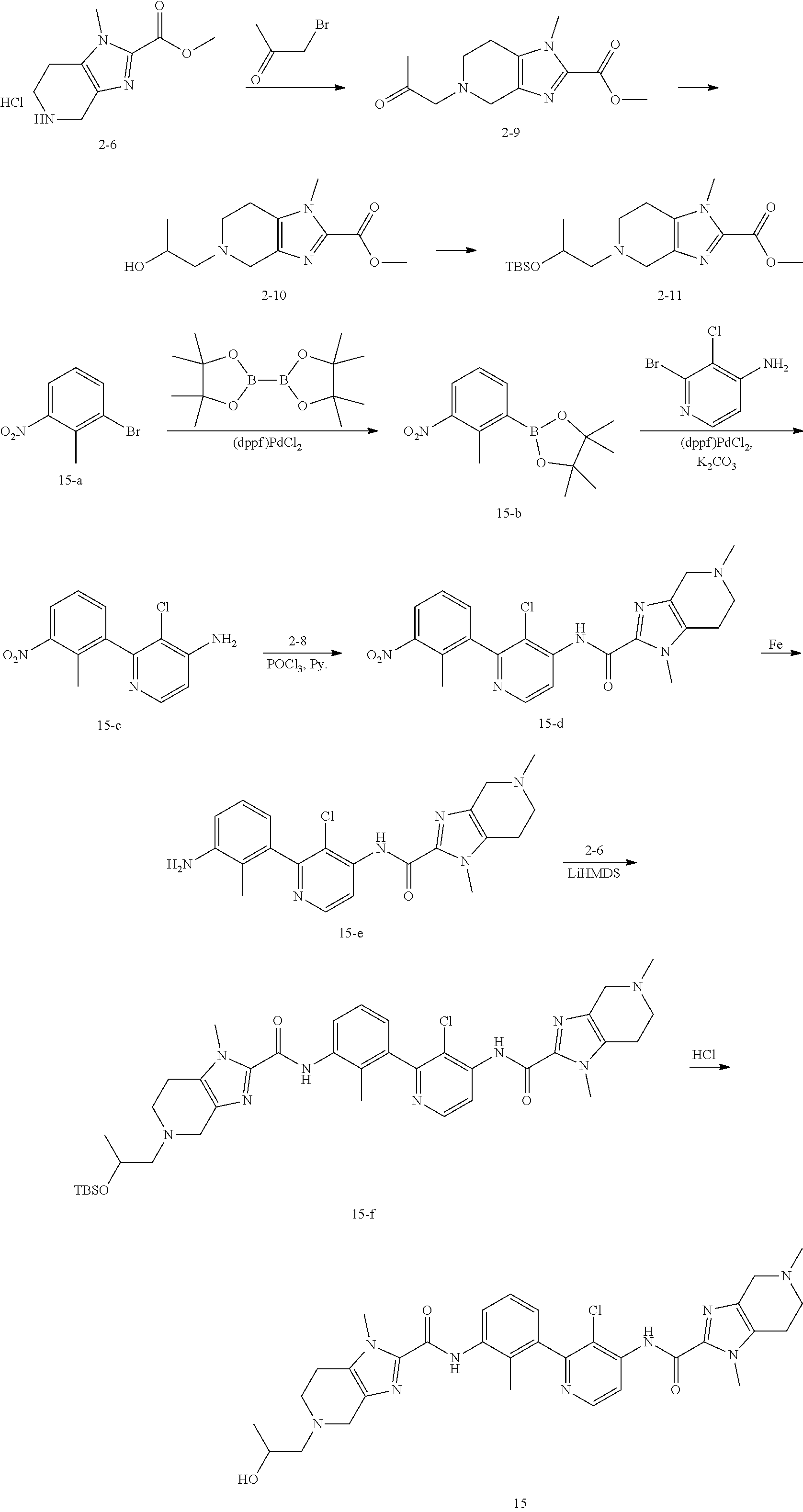

### Step 1 5-(tert-butyl) 2-methyl 1-methyl-1,4,6,7-tetrahydro-5H-imidazo[4,5-c]pyridine-2,5-dicarboxylate 2-5

N-butyllithium (1.6 M in THF, 58 mL, 94.3 mmol) was added dropwise to a solution of compound 2-4 (14.9 g, 0.06 mol) in tetrahydrofuran (50 ml) at −78° C., followed by stirring at a temperature of from −30° C. to −20° C. for 30 min. The reaction solution was then cooled to −78° C., and methyl chloroformate (11.8 g, 126 mmol) was added dropwise to the reaction solution. The reaction solution was kept at −78° C. and stirred for 2 h. TLC dot plate showed that the reaction was complete. Saturated ammonium chloride solution was added dropwise to the reaction solution to quench the reaction. The reaction solution was filtered through diatomite to remove inorganic salts. The filtrate was extracted twice with ethyl acetate (300 mL×2), and the organic phase was dried and concentrated to obtain a crude product. The crude product was separated by silica gel column (petroleum ether:ethyl acetate=10:1 to 3:1) to obtain a yellow solid compound, which was then pulped with petroleum ether:ethyl acetate=10:1 to obtain a white solid compound 2-5 (3.0 g).

¹H NMR (400 MHz, CDCl3) δ 4.49 (s, 2H), 3.93 (s, 3H), 3.88 (s, 3H), 3.82-3.72 (m, 2H), 2.72-2.62 (m, 2H), 1.46 (s, 9H).

### Step 2 1-methyl-4,5,6,7-tetrahydro-1H-imidazo[4,5-c]pyridine-2-carboxylic acid hydrochloride 2-6

A solution of compound 2-5 (3.0 g, 10 mmol) in methanolic hydrochloric acid solution (3.0 M, 40 mL) was stirred at room temperature overnight. LCMS showed that the raw material was completely reacted. The reaction solution was directly dried by rotary evaporation to obtain crude compound 2-6 (2.8 g), which was directly used in the next reaction.

LCMS (ESI): m/z 196.0 (M+H)⁺.

### Step 3 methyl 1,5-dimethyl-4,5,6,7-tetrahydro-1H-imidazo[4,5-c]pyridine-2-carboxylate 2-7

Formaldehyde (0.7 mL, 37 wt. % aqueous solution) was added to compound 2-6 (1.3 g, 5.6 mmol) in methanol/dichloroethane (10 mL/30 mL), and the reaction solution was stirred at room temperature for 30 min. Then sodium borohydride acetate (3.5 g, 16.8 mmol) was added to the reaction system, and the reaction system was kept stirring at room temperature for 2 h. The reaction solution was directly concentrated until dried. The obtained crude product was dispersed in water, and extracted with ethyl acetate. The organic phase was dried over sodium sulfate, and the filtrate was concentrated to obtain a crude product, which was separated by silica gel column chromatography (dichloromethane:methanol=100:1 to 10:1) to obtain compound 2-7 (0.90 g, yield: 75%).

LCMS (ESI): m/z 210.2 (M+H)+;

¹H NMR (400 MHz, CDCl3) δ 3.92 (s, 3H), 3.86 (s, 3H), 3.50 (s, 2H), 2.79 (t, J=5.5 Hz, 2H), 2.69 (dd, J=8.6, 3.2 Hz, 2H), 2.51 (s, 3H).

### Step 4 lithium 1,5-dimethyl-4,5,6,7-tetrahydro-1H-imidazo[4,5-c]pyridine-2-carboxylate 2-8

Lithium hydroxide monohydrate (199 mg, 4.73 mmol) was added to a solution of compound 2-7 (0.9 g, 4.30 mmol) in methanol (10 ml), and the reaction solution was kept at room temperature for 16 h. The reaction solution was directly dried by rotary evaporation to obtain yellow solid compound 2-8 (0.95 g, yield: 100%).

LCMS (ESI): m/z 196.3 (M+H)+.

### Step 5 methyl 1-methyl-5-(2-oxopropyl)-4,5,6,7-tetrahydro-1H-imidazo[4,5-c]pyridine-2-carboxylate 2-9

Bromoacetone (1.5 g, 11.2 mmol) and triethylamine (3.2 mL, 22.4 mmol) was added to a solution of compound 2-6 (1.3 g, 5.6 mmol) in dioxane (20 mL). The reaction solution was kept at 80° C. and stirred for 2 h. TLC (dichloromethane:methanol=10/1) detection showed that the reaction was complete. The reaction solution was directly concentrated until dried. The concentrate was diluted with water (20 mL) and extracted with ethyl acetate (100 mL×2). The organic phase was concentrated to obtain a crude product. The crude product was separated by silica gel column to obtain compound 2-9 (1.5 g).

LCMS (ESI): m/z 252.2 (M+H)+;

1H NMR (400 MHz, CDCl3) δ 3.92 (s, 3H), 3.87 (s, 3H), 3.67 (s, 2H), 3.48 (s, 2H), 2.93 (t, J=5.7 Hz, 2H), 2.73 (t, J=5.7 Hz, 2H), 2.19 (s, 3H).

### Step 6 5-(2-hydroxypropyl)-1-methyl-4,5,6,7-tetrahydro-1H-imidazo[4,5-c]pyridine-2-carboxylate 2-10

Sodium borohydride (0.23 g, 5.9 mmol) was added in batches to a solution of compound 2-9 (3.0 g, 11.9 mmol) in methanol (30 mL) at 0° C., and the reaction solution was stirred at 25° C. for 2 h. LCMS showed that the starting material was completely reacted. Water (30 ml) was added to the reaction solution to quench the reaction, and the mixture was extracted with dichloromethane (100 mL×2). The organic phase was dried over anhydrous sodium sulfate, and the filtrate was concentrated to obtain a crude product. The crude product was purified by silica gel column (dichloromethane:methanol=100/1 to 10/1) to obtain compound 2-10 (0.80 g) as a yellow solid.

LCMS (ESI): m/z 254.2 (M+H)⁺.

### Step 7 methyl 5-(2-(((tert-butyldimethylsilyl)oxy)propyl)-1-methyl-4,5,6,7-tetrahydro-1H-imidazo[4,5-c]pyridine-2-carboxylate 2-11

To a solution of compound 2-10 (0.8 g, 3.2 mmol) in DMF (15 mL) was added imidazole (0.6 g, 9.3 mmol) followed by TBSCI (0.9 g, 6.2 mmol). The reaction solution was kept stirring at room temperature overnight. Water (500 mL) and ethyl acetate (100 mL) were added to the reaction solution. The organic phase was dried over anhydrous sodium sulfate, and the filtrate was dried by rotary evaporation to obtain a crude product. The crude product was purified by silica gel column chromatography (petroleum ether:ethyl acetate=1:1) to obtain yellow solid compound 2-11 (0.6 g, yield: 50%).

LCMS (ESI): m/z 368.2 (M+H)⁺;

1H NMR (400 MHz, CDCl3) δ 3.92 (d, J=6.0 Hz, 1H), 3.86 (s, 3H), 3.80 (s, 3H), 3.51 (s, 2H), 2.83 (dt, J=7.7, 5.7 Hz, 2H), 2.61-2.39 (m, 4H), 1.12 (d, J=6.1 Hz, 3H), 0.88-0.78 (m, 9H), 0.01-0.03 (m, 6H).

### Step 8 4,4,5,5-tetramethyl-2-(2-methyl-3-nitrophenyl)-1,3,2-dioxaborane 15-b

Compound 15-a (5.0 g, 23.3 mmol), diboron pinacol ester (8.9 g, 34.9 mmol), potassium acetate (6.8 g, 69.9 mmol), Pd(dppf)Cl₂ (0.8 g, 1.16 mmol) and dioxane (100 mL) were respectively added to a reaction flask. The atmosphere was replaced with nitrogen for three times, and then the temperature was raised to 100° C., and reaction was performed for 12 h. Water (20 ml) and ethyl acetate (100 mL×2) were added to the reaction solution, and the organic phase was dried and concentrated to obtain a crude product. The crude product was purified by silica gel column (ethyl acetate/petroleum ether=0 to 50%) to obtain compound 15-b (2.3 g, yield 45%).

### Step 9 3-chloro-2-(2-methyl-3-nitrophenyl)pyridin-4-amine 15-c

Compound 15-b (720 mg, 2.74 mmol), 2-bromo-3-chloropyridin-4-amine (470 mg, 2.28 mmol), potassium carbonate (630 mg, 4.56 mmol), Pd(dppf)Cl₂ (210 mg, 0.3 mmol) and dioxane/water (10 mL/2 ml) were respectively added to a reaction flask. The atmosphere was replaced with nitrogen for three times. The system was heated to 80° C. and reacted for 4 h. LCMS showed that the starting material was completely reacted. Water (30 ml) and ethyl acetate (100 mL×2) were added to the reaction solution. The organic phase was dried and concentrated to obtain a crude product, which was purified by silica gel column chromatography (ethyl acetate/petroleum ether=0 to 30%) to obtain a brown solid compound 15-c (605 mg, yield: 84%).

LCMS (ESI): m/z 264.0 (M+H)⁺.

### Step 10 N-(3-chloro-2-(2-methyl-3-nitrophenyl)pyridin-4-yl)-1,5-dimethyl-4,5,6,7-tetrahydro-1H-imidazo[4,5-c]pyridine-2-carboxamide 15-d

Compound 15-c (150 mg, 0.57 mmol) and compound 2-8 (144 mg, 0.74 mmol) were dissolved in pyridine (2 mL)/dichloromethane (2 ml). POCl₃ (174 mg, 1.14 mmol) was added dropwise to the above reaction system at room temperature. The reaction solution was kept at room temperature for 3 hours. LCMS showed that the starting material was completely reacted. The reaction solution was concentrated to remove dichloromethane, diluted with saturated sodium bicarbonate solution (50 ml), and extracted with ethyl acetate (50 mL×2). The organic phase was dried over anhydrous sodium sulfate, and the filtrate was concentrated until dried to obtain the crude product, which was separated by silica gel column chromatography (methanol/dichloromethane=0% to 10%) to obtain a yellow solid compound 15-d (200 mg, yield: 45%).

LCMS (ESI): m/z 441.1 (M+H)⁺.

### Step 11 N-(2-(3-amino-2-methylphenyl)-3-chloropyridin-4-yl)-1,5-dimethyl-4,5,6,7-tetrahydro-1H-imidazo[4,5-c]pyridine-2-carboxamide 15-e

Compound 15-d (200 mg, 0.45 mmol), iron powder (152 mg, 2.72 mmol), saturated ammonium chloride solution (1 ml), and ethanol (4 ml) were successively added to a reaction flask, and the reaction was carried out at 60° C. for 3 hours. LCMS showed that the starting material was completely reacted. The reaction solution was filtered through diatomite. The filter cake was washed with ethanol (20 ml*3). The combined filtrate was dried by rotary evaporation to obtain a crude product, which was purified by silica gel column chromatography (methanol/dichloromethane=0 to 10%) to obtain a yellow solid compound 15-e (180 mg, yield: 96%).

LCMS (ESI): m/z 411.1 (M+H)⁺.

### Step 12 5-(2-((tert-butyldimethylsilyl)oxy)propyl)-N-(3-(3-chloro-4-(1,5-dimethyl-4,5,6,7-tetrahydro-1H-imidazo[4,5-c]pyridine-2-carboxamidopyridin-2-yl)-2-methylphenyl)-1-methyl-4,5,6,7-tetrahydro-1H-imidazo[4,5-c]pyridine-2-carboxamide 15-f

LiHMDS (1.1 ml, 1.17 mmol) was added dropwise to a solution of compound 15-e (160 mg, 0.39 mmol) and compound 2-11 (286 mg, 0.78 mmol) in anhydrous toluene (6 mL), and the reaction solution was kept at room temperature under stirring overnight. LCMS detection showed that the main point is the product. Ammonium chloride (10% w, 10 ml) was added dropwise to the reaction solution, followed by extraction with ethyl acetate (20 ml*2). The organic phase was dried over anhydrous sodium sulfate, and the filtrate was concentrated until dried to obtain a crude product. The crude product was isolated by a preparative plate to obtain compound 15-f as a brown solid (180 mg, yield: 62%).

LCMS (ESI): m/z 746.0 (M+H)⁺.

### Step 13 N-(3-chloro-2-(3-(5-(2-hydroxypropyl)-1-methyl-4,5,6,7-tetrahydro-1H-imidazo[4,5-c]pyridine-2-carboxamido))-2-methylphenyl)pyridin-4-yl)-1,5-dimethyl-4,5,6,7-tetrahydro-1H-imidazo[4,5-c]pyridine-2-carboxamide 15

Compound 15-f (180 mg, 0.24 mmol) was dissolved in ethyl acetate hydrochloride (6 mL)/methanol hydrochloride (2.5 ml), and the reaction solution was stirred at room temperature for 3 h. LCMS showed the reaction was complete. The reaction solution was directly dried by rotary evaporation. The obtained concentrate was diluted with methanol (3 ml). The pH of the methanol solution was adjusted to 7 with triethylamine. After that, the resultant was purified by prep-HPLC (Instrument: Gilson Flow: 25 mL/min Mobile phase A: 0.1% NH₄OH in water; Mobile phase B: ACN Gradient: 40%-50%) to obtain solid product 15 (60.5 mg, yield 40%).

LCMS (ESI): m/z 632.6 (M+H)⁺;

¹H NMR (400 MHz, CDCl3) δ 10.07 (s, 1H), 9.18 (s, 1H), 8.52-8.48 (m, 2H), 8.15 (d, J=7.6 Hz, 1H), 7.34 (t, J=7.9 Hz, 1H), 7.09 (d, J=6.8 Hz, 1H), 3.98 (s, 7H), 3.84-3.57 (m, 4H), 3.20-2.72 (m, 8H), 2.70-2.60 (m, 4H), 2.58-2.45 (m, 1H), 2.13 (s, 3H), 1.19 (d, J=6.1 Hz, 3H).

### Example 16

### N-(3-(3-chloro-2-(1,5-dimethyl-4,5,6,7-tetrahydro-1H-imidazo[4,5-c]pyridine-2-carboxamido)pyridin-4-yl)-2-methylphenyl)-5-(2-hydroxypropyl)-1-methyl-4,5,6,7-tetrahydro-1H-imidazo[4,5-c]pyridine-2-carboxamide

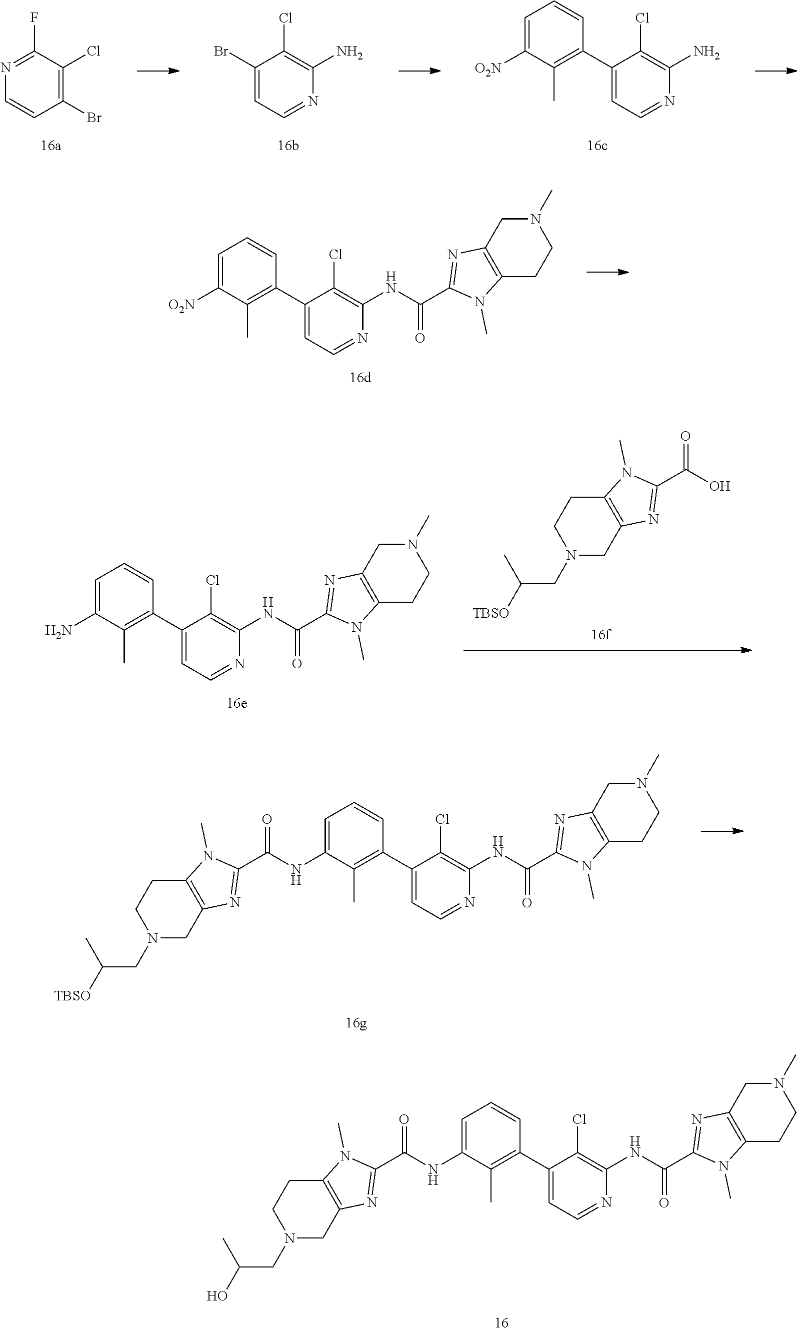

### Step 1 4-bromo-3-chloropyridin-2-amine 16b

Compound 16a (500 mg, 2.38 mmol) was added to ammonia water (28%, 4 mL), heated to 120° C. and stirred for 2 h. The ammonia aqueous solution was removed by rotary evaporation to obtain the target compound 16b (475 mg, yield 96%) as a white solid.

### Step 2 3-chloro-4-(2-methyl-3-nitrophenyl)pyridin-2-amine 16c

Compound 16b (500 mg, 2.38 mmol), 4,4,5,5-tetramethyl-2-(2-methyl-3-nitrophenyl)-1,3,2-dioxaborane (711 mg, 2.70 mmol), K₂CO₃ (657 mg, 4.76 mmol) and Pd(dppf)Cl₂ (0.17 g, 0.24 mmol) were added to a mixed solvent of dioxane/H₂O (5 ml/1 mL), which was protected by nitrogen, heated to 80° C. and stirred for 4 h. After cooling to room temperature, 20 mL of water was added, and the mixture was extracted with ethyl acetate (40 ml×2). The organic phases were combined, concentrated, and purified by column chromatography (EA/PE=0%-100%) to obtain the target compound 16c (508 mg, yield 81%) as a brown solid.

### Step 3 N-(3-chloro-4-(2-methyl-3-nitrophenyl)pyridin-2-yl)-1,5-dimethyl-4,5,6,7-tetrahydro-1H-imidazo[4,5-c]pyridine-2-carboxamide 16d

Compound 16c (200 mg, 0.76 mmol), 1,5-dimethyl-4,5,6,7-tetrahydro-1H-imidazo[4,5-c]pyridine-2-carboxylic acid (192 mg, 0.98 mmol) and phosphorus oxychloride (234 mg, 1.52 mmol) were added to a mixed solvent of pyridine/DCM (2 mL/2 ml), stirred at room temperature for 2 h, and detected by LCMS to determine the end of the reaction. After concentrating to remove the solvent, 10 mL of saturated NaHCO₃ aqueous solution was added, and it was extracted with ethyl acetate (50 mL×2). The organic phases were combined, dried with Na₂SO₄, and concentrated to obtain a crude brown oil, which was further purified by silica gel column chromatography (methanol/DCM=0% to 10%) to obtain the target compound 16d (110 mg, yield 32.8%) as a yellow solid.

MS m/z (ESI): 441.1[M+1]+.

### Step 4 N-(4-(3-amino-2-methylphenyl)-3-chloropyridin-2-yl)-1,5-dimethyl-4,5,6,7-tetrahydro-1H-imidazo[4,5-c]pyridine-2-carboxamide 16e

Compound 16d (110 mg, 0.25 mmol), iron powder (84 mg, 1.5 mmol) and saturated NH₄Cl aqueous solution (1 mL) were added to EtOH (4 mL), heated to 60° C., stirred for 3 h, and detected by LCMS to determine the end of the reaction. After cooling to room temperature, the reaction solution was filtered through diatomite. The filtrate was concentrated to remove ethanol and extracted with ethyl acetate (5 ml×2). The organic phases were combined, dried over Na₂SO₄, concentrated, and purified by silica gel column chromatography (methanol/DCM=0 to 10%) to obtain the target compound 16e (85 mg, yield 82.9%) as a yellow solid.

MS m/z (ESI): 411.4[M+1]⁺.

### Step 5 5-(2-((tert-butyldimethylsilyl)oxy)propyl)-N-(3-(3-chloro-2-(1,5-dimethyl-4,5,6,7-tetrahydro-1H-imidazo[4,5-c]pyridine-2-carboxamidopyridin-4-yl)-2-methylphenyl)-1-methyl-4,5,6,7-tetrahydro-1H-imidazo[4,5-c]pyridine-2-carboxamide 16g

Compound 16e (85 mg, 0.21 mmol) and compound 16f (114 mg, 0.32 mmol) were dissolved in toluene (2 mL). LiHMDS (0.62 ml, 0.62 mmol) was added under stirring. The mixture was stirred at room temperature overnight. The reaction was quenched by adding 10% NH₄Cl aqueous solution (5 mL) and it was extracted with ethyl acetate (20×2 mL). The organic phases were combined, dried over Na₂SO₄, concentrated, and purified by pre-TLC to obtain the target compound 16g (110 mg, yield 71%) as a brown solid.

MS m/z (ESI): 746.4[M+1]+.

### Step 6 N-(3-(3-chloro-2-(1,5-dimethyl-4,5,6,7-tetrahydro-1H-imidazo[4,5-c]pyridine-2-carboxamido)pyridin-4-yl)-2-methylphenyl)-5-(2-hydroxypropyl)-1-methyl-4,5,6,7-tetrahydro-1H-imidazo[4,5-c]pyridine-2-carboxamide 16

Compound 16g (110 mg, 0.24 mmol) was dissolved in DCM (2 mL), added with TBAF (0.5 mL, 1 M in THF), and stirred at room temperature for 16 h. The end of the reaction was determined by LCMS detection. The solvent was removed by rotary evaporation. The crude product was further purified by prep-HPLC (Instrument: Gilson Flow: 25 mL/min Mobile phase A: 0.1% NH₄OH in water, Mobile phase B: ACN, Gradient: 40%-50%) to obtain compound 16 (32.7 mg, yield: 35%) with a purity of 100% (254 nm) as a white solid.

MS m/z (ESI): 632.6[M+1]+

1H NMR (400 MHz, CDCl3) δ 10.11 (s, 11H), 9.19 (s, 1H), 8.49 (d, J=4.8 Hz, 1H), 8.15 (d, J=8.0 Hz, 1H), 7.35 (t J=7.8 Hz, 1H), 7.01-6.96 (m, 2H), 4.01 (s, 3H) 0.4.00 (s, 3H), 3.81-3.59 (m, 4H), 3.08 (m, 1H), 2.95-2.7 (m, J=7H), 2.63 (s, 3H), 2.51 (m, 1H), 2.13 (s, 3H), 1.21 (d, J=6.0 Hz, 3H).

### Example 17

### (R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[4,5-b]pyridin-5-yl)methyl)pyrrolidine-3-carboxylic acid trifluoroacetate

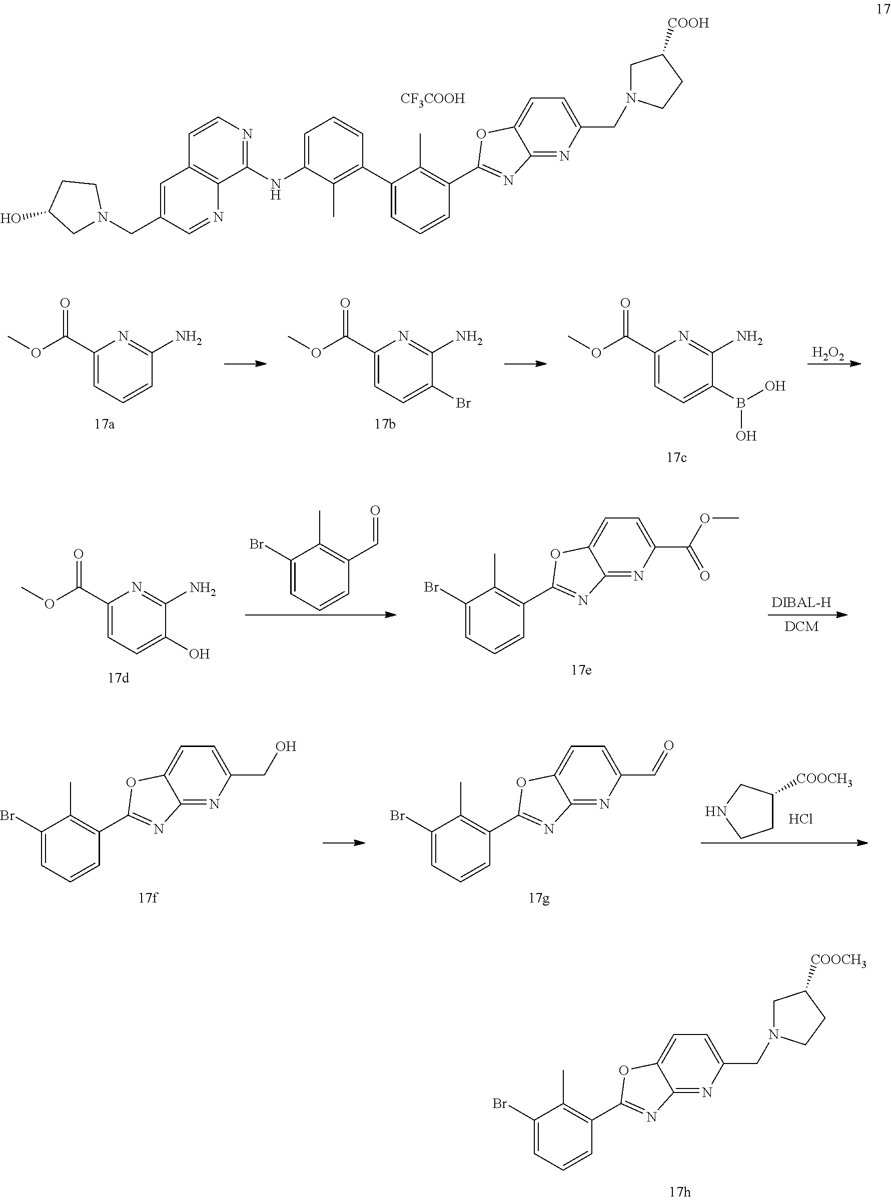

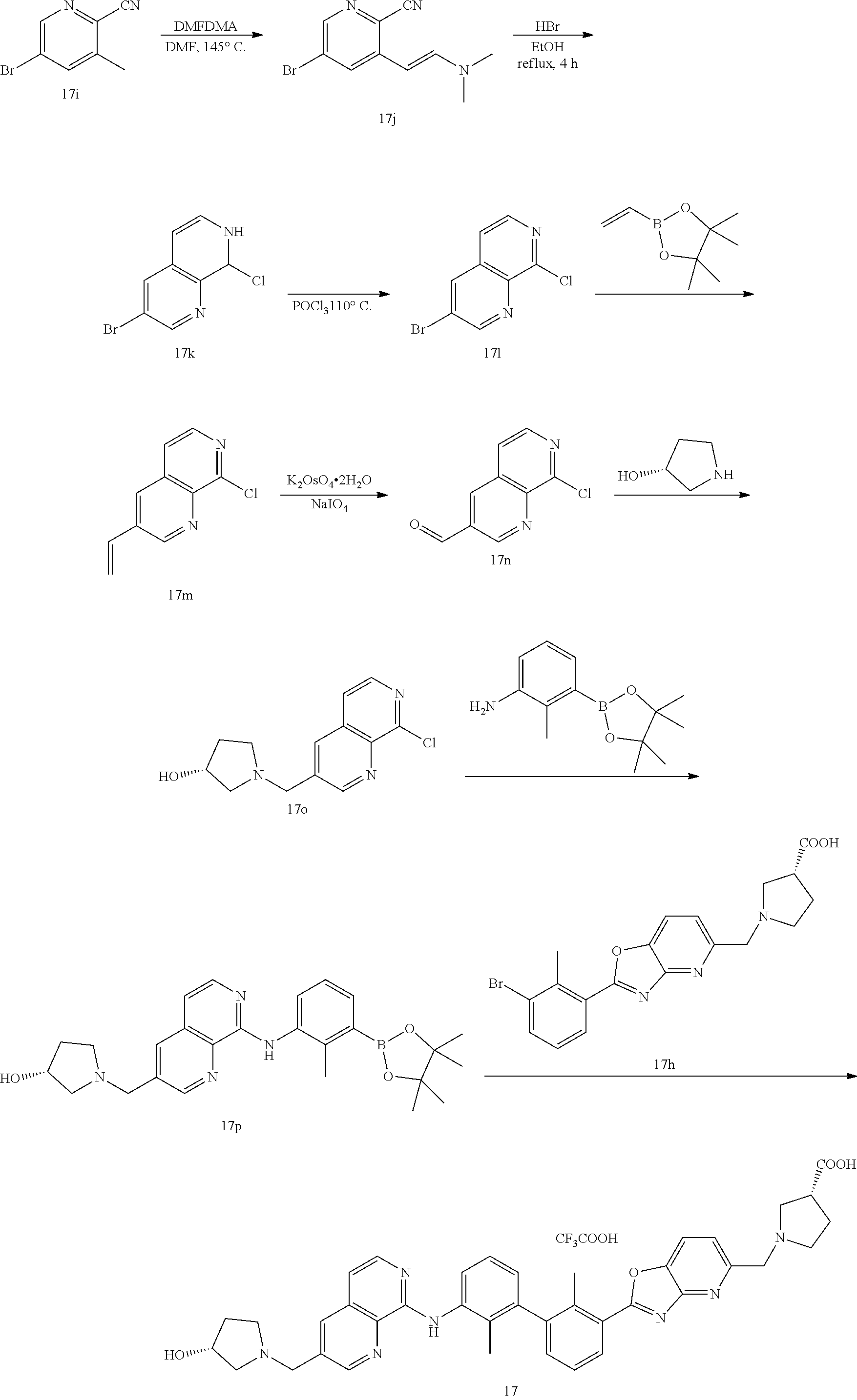

### Step 1 methyl 6-amino-5-bromopicolinate 17b

Compound methyl 6-aminopicolinate (50 g, 328.9 mmol) was dissolved in DCM (500 mL). Bromine (16.9 mL, 328.9 mmol) was dissolved in DCM (200 mL). Bromine in DCM was slowly added into the compound solution dropwise under ice bath condition. After the addition, the reaction was carried out at room temperature for 12 h. 150 mL of saturated sodium thiosulfate solution was added, and the mixture was extract with dichloromethane three times. The organic phases were combined, dried with anhydrous sodium sulfate, concentrated and purified by column chromatography to obtain the title compound 17b (15.2 g, 65.5 mmol) with a yield of 19.9%.

### Step 2 (2-amino-6-(methoxycarbonyl)pyridin-3-yl)boronic acid 17c

Compound methyl 6-amino-5-bromopicolinate (17b) (15.2 g, 65.5 mmol) and diboron pinacol ester (30.8 g, 131 mmol) were dissolved in 1,4-dioxane (100 mL). [1,1′-Bis(diphenylphosphino)ferrocene]dichloride palladium (2.39 g, 3.2 mmol) and potassium acetate (192 g, 196.5 mmol) were added, and the reaction was carried out at 100° C. for 6 hours under argon protection. The resultant was filtered to remove the solid, and the reaction solution was dried by rotary evaporation to obtain a crude product of the target compound (21.6 g).

### Step 3 methyl 6-amino-5-hydroxypicolinate 17d

The crude compound (2-amino-6-(methoxycarbonyl)pyridin-3-yl)boronic acid (17c) was dissolved in tetrahydrofuran (100 mL), and 30% aqueous hydrogen peroxide solution (15 mL) was added dropwise under an ice bath and reacted for 3 hours. Sodium sulfite solution was added to quench the reaction. Part of tetrahydrofuran was removed by rotary evaporation. The resultant was recrystallized to obtain the target compound 17d, methyl 6-amino-5-hydroxypicolinate (8.8 g, 52.1 mmol), with a two-step yield of 79%.

### Step 4 methyl 2-(3-bromo-2-methylphenyl)oxazolo[4,5-b]pyridine-5-carboxylate 17e

Compound methyl 6-amino-5-hydroxypicolinate (17d) (8.8 g, 52.1 mmol) and compound 3-bromo-2-methylbenzaldehyde (20.8 g, 104.2 mmol) were dissolved in 80 mL of a mixed solvent of ethyl acetate and ethanol (v/v=1:1), which were stirred at 60° C. for 6 hours, and dried by rotary evaporation to remove the solvent. Dichloromethane (100 mL) was added, and then 2,3-dichloro-5,6 dicyano-p-benzoquinone (17.8 g, 78.5 mmol) was added, which were reacted at room temperature for 5 hours. Water was added, and the mixture was extracted 3 times with dichloromethane. The organic phases were combined, dried over anhydrous sodium sulfate, concentrated and purified by column chromatography to obtain the title compound 17e (2.1 g, 6.1 mmol) with a yield of 11%.

### Step 5 (2-(3-bromo-2-methylphenyl)oxazolo[4,5-b]pyridin-5-yl)methanol 17f

Compound methyl 2-(3-bromo-2-methylphenyl)oxazolo[4,5-b]pyridine-5-carboxylate (17e) (2.1 g, 6.1 mmol) was dissolved in dichloromethane (50 mL). The atmosphere was replaced with argon. Diisobutylaluminum hydride (1.86 g, 12.2 mmol) was added dropwise at −78° C. The system was slowly warmed to room temperature, and reacted for 1 hour. Ammonium chloride solution was added and stirred for 10 min. Water was added, and the mixture was extracted 3 times with ethyl acetate, dried over anhydrous sodium sulfate, concentrated and purified by column chromatography to obtain the title compound 17f (1.7 g, 5.4 mmol) with a yield of 88%.

### Step 6 2-(3-bromo-2-methylphenyl)oxazolo[4,5-b]pyridine-5-formaldehyde 17g

Compound (2-(3-bromo-2-methylphenyl)oxazolo[4,5-b]pyridine-5-yl)methanol (17f) (1.7 g, 5.4 mmol) and Dess-Martin reagent (4.5 g, 10.8 mmol) was dissolved in dichloromethane (50 mL) and reacted at room temperature for 3 h. Sodium thiosulfate solution was added, and stirred for 10 minutes. The system was added and extracted with ethyl acetate 3 times, dried with anhydrous sodium sulfate, concentrated and purified by column chromatography to obtain the title compound 17g (1.57 g, 4.9 mmol) with a yield of 92%.

### Step 7 methyl (R)-1-(((2-(3-bromo-2-methylphenyl)oxazolo[4,5-b]pyridin-5-yl)methyl)pyrrolidine-3-carboxylate 17h

Compound 2-(3-bromo-2-methylphenyl)oxazolo[4,5-b]pyridine-5-formaldehyde (17g) (1.57 g, 4.9 mmol) and compound methyl (R)-pyrrolidine-3-carboxylate hydrochloride (2.4 g, 14.7 mmol) were dissolved in dichloromethane (50 mL) and stirred at room temperature for 1 h. After that, sodium triacetoxyborohydride (3.1 g, 14.7 mmol) was added, and reacted at room temperature for 12 h. The reaction was quenched by adding saturated sodium bicarbonate solution, extracted 3 times with 30 mL dichloromethane, dried over anhydrous sodium sulfate, concentrated and purified by column chromatography to obtain the title compound 17h (1.4 g, 3.3 mmol) with a yield of 67%.

### Step 8 (E)-5-bromo-3-(2-(dimethylamino)vinyl)-2-cyanopyridine 17j

Compound 5-bromo-3-methyl-2-cyanopyridine (17i) (30 g, 152 mmol) and compound N,N-dimethylformamide dimethyl acetal (36 g, 304 mmol) were dissolved in N,N-dimethylformamide (100 mL), heated to 140° C. and reacted for 20 h. The solvent was then dried by rotary evaporation. Ethyl acetate was added to the sample mixture and it was purified by column chromatography to obtain the title compound 17j (18 g, 91.3 mmol) with a yield of 60%.

### Step 9 3-bromo-1,7-naphthyridin-8-(7H)-one 17k

Compound (E)-5-bromo-3-(2-(dimethylamino)vinyl)-2-cyanopyridine (17j) (18 g, 91.3 mmol) was dissolved in ethanol (80 mL), and hydrobromic acid (23 g, 285 mmol) was added, which were heated to 90° C., reacted for 6 h, and filtered to obtain a yellow solid. Hydrobromic acid was used to wash off sodium bicarbonate, such that the title compound (6.8 g, 30.2 mmol) was obtain with a yield of 33.1%.

### Step 10 3-bromo-8-chloro-1,7-naphthyridine 17l

Compound 3-bromo-1,7-naphthyridin-8-(7H)-one (6.8 g, 30.2 mmol) was dissolved in phosphorus oxychloride (50 mL), heated to 110° C. and reacted for 3 h. The reaction solution was dropped into ice water, and extracted three times with ethyl acetate. The organic phase was dried over anhydrous sodium sulfate, and purified by column chromatography to obtain the title compound 17l (4.1 g, 16.8 mmol) with a yield of 55.6%.

### Step 11 8-chloro-3-vinyl-1,7-diazanaphthalene 17m

[1,1′-Bis(diphenylphosphino)ferrocene]palladium dichloride (601 mg, 0.823 mmol) and sodium carbonate (1.744 g, 16.46 mmol) were added to compound 17l (2 g, 8.23 mmol) and 4,4,5,5-tetramethyl-2-vinyl-1,3,2-dioxaborane (1.394 g, 9.053 mmol) in 1,4-dioxane/water (v/v=4/1) (50 mL), reacted at 110° C. for 4 hours under argon protection until the reaction was finished. The reaction solution was added with water, extracted three times with ethyl acetate, washed with saturated brine, dried over anhydrous sodium sulfate, filtered, concentrated and purified by column chromatography to obtain the title compound 17m (800 mg, 4.21 mmol) with a yield of 51%.

### Step 12 8-chloro-1,7-diazanaphthalene-3-formaldehyde 17n

Potassium osmate (31 mg, 0.0842 mmol) was added to compound 8-chloro-3-vinyl-1,7-naphthalene (17m) (800 mg, 4.21 mmol) in 1,4-dioxane/water (v/v=4/1) (50 mL) under ice bath condition and stirred for 1 h. After that, sodium periodate (1.8 g, 8.42 mmol) was added, and the mixture was heated to room temperature and stirred overnight until the reaction was finished. The reaction solution was added with water, extracted three times with ethyl acetate, washed with saturated brine, dried over anhydrous sodium sulfate, filtered, concentrated and purified by column chromatography to obtain the title compound 17n (600 mg, 3.125 mmol) with a yield of 74%.

### Step 13 (R)-1-((8-chloro-1,7-diazanaphthalen-3-yl)methyl)pyrrolidin-3-ol 17o

Compound 8-chloro-1,7-naphthalene-3-formaldehyde (17n) (800 mg, 4.16 mmol) and (R)-pyrrolidin-3-ol 7q (1.0879 g, 12.513 mmol) were dissolved in dichloromethane (40 mL), added with acetic acid (250 mg, 0.416 mmol), and stirred for 1 h. Sodium triacetoxyborohydride (2.65 g, 12.513 mmol) was then added, and the mixture was stirred at room temperature overnight until the reaction was finished. The reaction solution was extracted with saturated sodium bicarbonate and ethyl acetate. The organic phase was washed with saturated brine, dried over anhydrous sodium sulfate, filtered, concentrated and purified by column chromatography to obtain the title compound 17o (0.72 g, 2.737 mmol) with a yield of 65%.

### Step 14 (R)-1-((8-((2-methyl-3-(4,4,5,5-tetramethyl-1,3,2-dioxaborolan-2-yl)phenyl)amino)-1,7-diazanaphthalen-3-yl)methyl)pyrrolidin-3-ol 17p

Compound (R)-1-((8-chloro-1,7-naphthalen-3-yl)methyl)pyrrolidin-3-ol (17o) (700 mg, 2.661 mmol) and compound 2-methyl-3-(4,4,5,5-tetramethyl-1,3,2-dioxaboran-2-yl)aniline (682 mg, 2.927 mmol) were dissolved in isopropanol (40 mL), and added with HCl/dioxane (1.33 mL, 5.322 mmol). The reaction solution was heated to 95° C., stirred for 4 h, and dried by rotary evaporation. Saturated sodium bicarbonate solution was added, and the resultant was extracted with ethyl acetate. The organic phase was washed with saturated brine, dried over anhydrous sodium sulfate, filtered, concentrated and purified by column chromatography to obtain the title compound 17p (0.65 g, 1.41 mmol) with a yield of 53%.

### Step 15 (R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[4,5-b]pyridin-5-yl)methyl)pyrrolidine-3-carboxylic acid trifluoroacetate 17

Compound (R)-1-((8-((2-methyl-3-(4,4,5,5-tetramethyl-1,3,2-dioxaborolan-2-yl)phenyl)amino)-1,7-diazanaphthalen-3-yl)methyl)pyrrolidin-3-ol (17p) (32 mg, 0.07 mmol) and compound methyl (R)-1-(((2-(3-bromo-2-methylphenyl)oxazolo[4,5-b]pyridin-5-yl)methyl)pyrrolidine-3-carboxylate (17h) (30 mg, 0.07 mmol) were dissolved in tert-butanol/water (v/v=2/1) (10 mL). 1,1′-Bis(di-cyclohexylphosphino)ferrocene palladium dichloride (5.1 mg, 0.007 mmol) and cesium carbonate (45 mg, 0.14 mmol) were added, heated to 100° C. and reacted for 2h. The resultant was filtered to remove the solid, and dried by rotary evaporation to remove the solvent. The target compound 17 (24.6 mg, 0.031 mmol) was obtained by preparative HPLC with a yield of 45%.

MS m/z (ESI): 669.7 [M+H]⁺.

¹H NMR (400 MHz, Methanol-d₄) δ 9.17 (d, J=2.1 Hz, 1H), 8.56 (d, J=2.1 Hz, 1H), 8.27 (dd, J=7.9, 1.6 Hz, 1H), 8.22 (d, J=8.3 Hz, 1H), 7.68-7.62 (m, 2H), 7.57 (s, 1H), 7.54 (d, J=7.6 Hz, 2H), 7.48 (dd, J=7.6, 1.5 Hz, 1H), 7.36 (d, J=7.5 Hz, 1H), 7.30 (d, J=7.0 Hz, 1H), 4.81 (s, 1H), 4.78-4.71 (m, 3H), 4.60 (s, 1H), 3.78-3.59 (m, 3H), 3.57-3.48 (m, 2H), 3.46 (q, J=1.6 Hz, 1H), 3.40 (dd, J =16.0, 6.5 Hz, 2H), 2.58 (s, 3H), 2.49-2.23 (m, 4H), 2.14 (d, J=5.5 Hz, 1H), 2.08 (s, 3H).

### Example 18

### (R)-1-((2-(2,2′-dimethyl-3′-(4,5,6,7-tetrahydrothiazolo[5,4-c]pyridine-2-carboxamido)-[1,1′-biphenyl]-3-yl)oxazolo[5,4-b]pyridin-6-yl)methyl)pyrrolidine-3-carboxylic acid trifluoroacetate

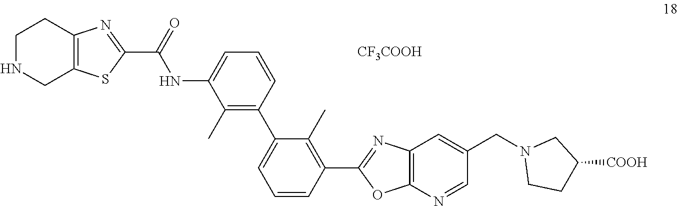 [SMILES: Cc1c(NC(=O)c2nc3c(s2)CNCC3)cccc1-c1cccc(-c2nc3cc(CN4CC[C@@H](C(=O)O)C4)cnc3o2)c1C ; "CF3COOH"]

A synthetic method similar to that of Example 4 was used to prepare the title product 18.

¹H NMR (400 MHz, DMSO-d₆) δ 10.41 (s, 1H), 9.43 (s, 2H), 8.53 (d, J=2.0 Hz, 1H), 8.46 (d, J=2.0 Hz, 1H), 8.14 (dd, J=8.0, 1.4 Hz, 1H), 7.57-7.45 (m, 2H), 7.38 (dd, J=7.6, 1.5 Hz, 1H), 7.32 (t, J=7.7 Hz, 1H), 7.07 (dd, J=7.7, 1.3 Hz, 1H), 4.59 (s, 2H), 4.51 (s, 2H), 3.51 (s, 4H), 3.23 (s, 2H), 3.09 (t, J=6.2 Hz, 2H), 2.40 (s, 3H), 2.20 (s, 1H), 1.92 (s, 3H).

MS m/z (ESI): 609.4 [M+H]⁺.

### Example 19

### (R)-1-(((7-chloro-2-(4-methyl-5-(2-methyl-3-(1-methyl-4,5,6,7-tetrahydro-1H-imidazole)[4,5-c]pyridine-2-carboxamido)phenyl)pyridin-3-yl)benzo[d]oxazol-5-yl)methyl)pyrrolidine-3-carboxylic acid

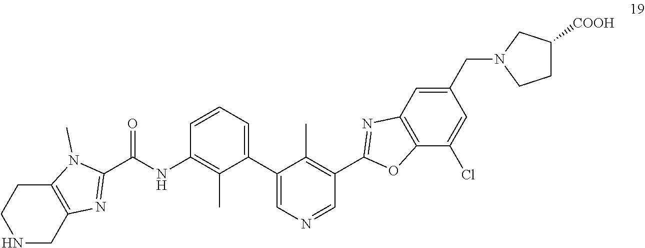 [SMILES: Cc1c(NC(=O)c2nc3c(n2C)CCNC3)cccc1-c1cncc(-c2nc3cc(CN4CC[C@@H](C(=O)O)C4)cc(Cl)c3o2)c1C]

A synthetic method similar to that of Example 9 was used to prepare the title product 19.

MS n/z (ESI):640.4 [M+I1]⁺

¹H NMR (400 MHz, Methanol-d₄) δ 9.33 (s. 1H), 8.52 (s, 1H), 7.97 (d, J=1.5 Hz, 1H), 7.79 (dd, J=8.1, 1.3 Hz, 1H), 7.73 (d, J=1.5 Hz, 1H), 7.40 (t, J=7.8 Hz, 1H), 7.13 (dd, J=7.7, 1.4 Hz, 1H), 4.57 (s, 2H), 4.26 (s, 2H), 3.98 (s, 3H), 3.76-3.36 (m, 7H), 3.03 (t, J=6.0 Hz, 2H), 2.62 (s, 3H), 2.53-2.27 (m, 2H), 2.06 (s, 3H).

### Example 20

### methyl (R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylate

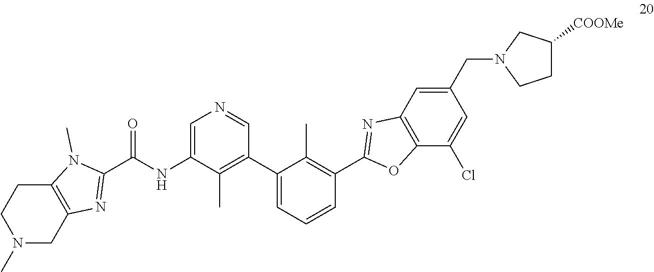 [SMILES: COC(=O)[C@@H]1CCN(Cc2cc(Cl)c3oc(-c4cccc(-c5cncc(NC(=O)c6nc7c(n6C)CCN(C)C7)c5C)c4C)nc3c2)C1]

A synthetic method similar to that of Example 8 was used to prepare the title product 20.

MS n/z (ESI): 668[M+1]⁺

¹H NMR (400 MHz, DMSO-d₆+D2O) δ 8.63 (s, 1H), 8.15 (s, 1H), 8.12 (dd, J=7.9, 1.4 Hz, 1H), 7.68 (d, J=1.5 Hz, 1H), 7.53 (t, J=7.7 Hz, 1H), 7.45 (d, J=1.4 Hz, 1H), 7.39 (dd, J=7.6, 1.5 Hz, 1H), 3.81 (s, 3H), 3.72-3.61 (m, 2H), 3.55 (s, 3H), 3.33 (s, 2H), 3.00 (d, J=1.9 Hz, 1H), 2.73-2.56 (m, 7H), 2.50 (dd, J=7.1, 2.2 Hz, 3H), 2.35 (d, J=10.2 Hz, 7H), 2.04-1.85 (m, 6H).

### Example 21

### (R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylic acid

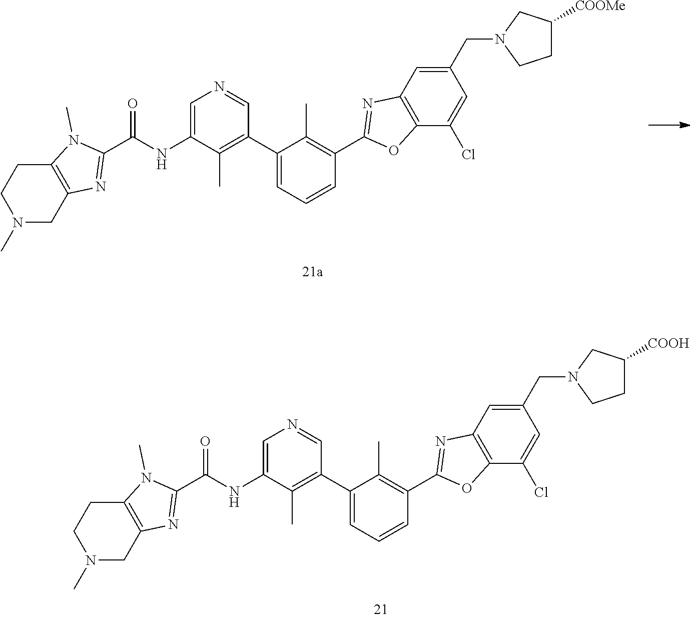 [SMILES: COC(=O)[C@@H]1CCN(Cc2cc(Cl)c3oc(-c4cccc(-c5cncc(NC(=O)c6nc7c(n6C)CCN(C)C7)c5C)c4C)nc3c2)C1 ; Cc1c(NC(=O)c2nc3c(n2C)CCN(C)C3)cncc1-c1cccc(-c2nc3cc(CN4CC[C@@H](C(=O)O)C4)cc(Cl)c3o2)c1C]

### Step 1 (R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylic acid

Compound 21a (60 mg, 0.089 mmol), MeOH/H₂O (1:1, 6.0 mL) and LiOH (6.2 mg, 0.27 mmol) were successively introduced into a single-necked bottle, and reacted at room temperature for 3 h. The system was neutralized to neutral with 1 N HCl solution, purified by reverse-phase column chromatography (C18, MeCN/H₂O) to obtain the title compound 21 (55 mg) as a white solid.

MS m/z (ESI): 654[M+1]⁺

¹H NMR (400 MHz, DMSO-d₆+D2O) δ 8.72 (s, 1H), 8.30 (s, 1H), 8.16 (dd, J=8.0, 1.4 Hz, 1H), 7.96 (d, J=1.5 Hz, 1H), 7.71 (d, J=1.5 Hz, 1H), 7.57 (t, J=7.8 Hz, 1H), 7.44 (dd, J=7.6, 1.4 Hz, 1H), 4.43 (d, J=41.6 Hz, 3H), 4.18 (s, 1H), 3.85 (s, 3H), 3.61-3.08 (m, 7H), 2.93 (s, 5H), 2.37 (s, 3H), 2.21 (s, 2H), 2.01 (s, 3H).

### Example 22

### (R)-1-((2-(3′-(5-((S)-2-hydroxypropyl)-4,5,6,7-tetrahydrothiazolo[5,4-c]pyridine-2-carboxamido)-2,2′-dimethyl-[1,1′-biphenyl]-3-yl)oxazolo[5,4-b]pyridin-6-yl)methyl)pyrrolidine-3-carboxylic acid

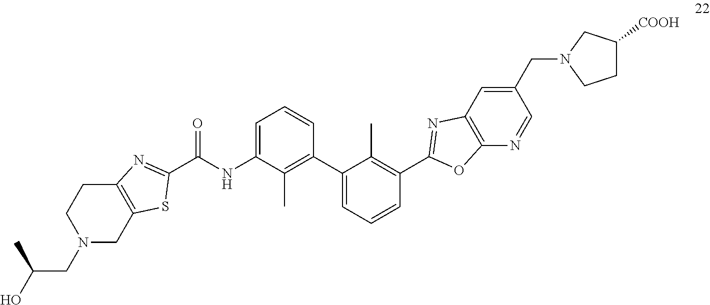 [SMILES: Cc1c(NC(=O)c2nc3c(s2)CN(C[C@H](C)O)CC3)cccc1-c1cccc(-c2nc3cc(CN4CC[C@@H](C(=O)O)C4)cnc3o2)c1C]

A synthetic method similar to that of Example 4 was used to prepare the title product 22.

MS m/z (ESI): 667.5[M+1]⁺

¹H NMR (400 MHz, Methanol-d₄) δ8.53 (d, J=2.0 Hz, 1H), 8.38 (d, J=2.1 Hz, 1H), 8.20 (dd, J=7.9, 1.5 Hz, 1H), 7.63 (dd, J=8.1, 1.3 Hz, 1H), 7.49 (t, J=7.7 Hz, 1H), 7.44-7.32 (m, 2H), 7.10 (dd, J=7.6, 1.3 Hz, 1H), 4.76 (s, 2H), 4.65 (s, 2H), 4.32-4.22 (m, 1H), 3.80 (s, 2H), 3.46 (d, J=33.7 Hz, 5H), 3.40-3.30 (m, 3H), 3.23 (dd, J=13.0, 10.5 Hz, 1H), 2.49 (s, 5H), 2.03 (s, 3H), 1.26 (d, J=6.2 Hz, 3H).

### Example 23

### (S)—N-(5-(2-chloro-3-(1-methyl-5-(oxetan-3-yl)-4,5,6,7-tetrahydro-1H-imidazo[4,5-c]pyridine-2-carboxamido)phenyl)-4-methylpyridin-3-yl)-5-(2-hydroxypropyl)-1-methyl-4,5,6,7-tetrahydro-1H-imidazo[4,5-c]pyridine-2-carboxamide

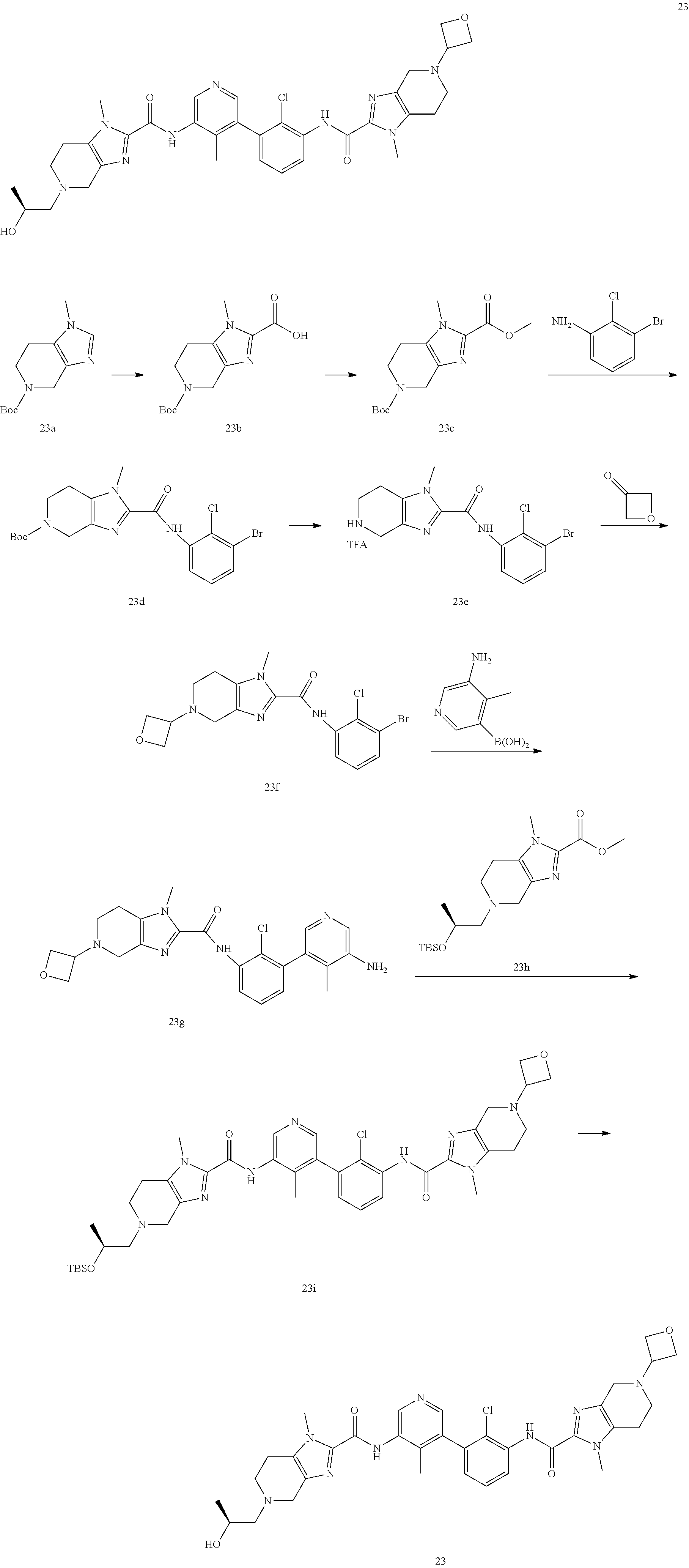

### Step 1 5-(tert-butoxycarbonyl)-1-methyl-4,5,6,7-tetrahydro-1H-imidazo[4,5-c]pyridine-2-carboxylic acid 23b

Compound 23a (5 g, 21.097 mmol) was dissolved in tetrahydrofuran (50 mL), and n-butyllithium (12.65 mL, 31.645 mmol) was slowly added dropwise under argon protection at −78° C. After stirring for 1 h, 10 g dry ice was added, and stirring was continued for 2 hours. The temperature was then slowly raised to room temperature. After that, dilute hydrochloric acid in dioxane was added to adjust the pH value to be weakly acidic. The reaction solution was dried by rotary evaporation and purified by column chromatography to obtain the title compound 23b (2.6 g, 9.252 mmol) with a yield of 43.8%.

MS m/z (ESI): 282.1[M+1]⁺.

### Step 2 5-(tert-butyl) 2-methyl 1-methyl-1,4,6,7-tetrahydro-5H-imidazo[4,5-c]pyridine-2,5-dicarboxylate 23c

Compound 23b (3 g, 10.676 mmol) was dissolved in N,N-dimethylformamide (30 mL), added with potassium bicarbonate (3.202 g, 32.028 mmol) and methyl iodide (3.032 g, 21.352 mmol), and reacted at room temperature for 24 h until the reaction was finished. The reaction solution was washed with water, extracted three times with dichloromethane, washed with saturated brine, dried over anhydrous sodium sulfate, filtered, concentrated and purified by column chromatography to obtain the title compound 23c (1 g, 3.389 mmol) with a yield of 31.36%.

MS m/z (ESI): 296.2 [M+H]⁺.

### Step 3 2-((3-bromo-2-chlorophenyl)carbamoyl)tert-butyl-1-methyl-1,4,6,7-tetrahydro-5H-imidazo[4,5-c]pyridine-5-carboxylate 23d

Compound 23c (1 g, 3.389 mmol) and 3-bromo-2-chloroaniline (1.047 g, 5.08 mmol) were dissolved in toluene (30 mL), and HMDSLi (6.778 mL, 6.778 mmol) was added at room temperature. After the addition, the reaction was carried out for 24 h until the reaction was finished. The reaction solution was washed with water, extracted three times with dichloromethane, washed with saturated brine, dried over anhydrous sodium sulfate, filtered, concentrated and purified by column chromatography to obtain the title compound 23d (1.1 g, 2.345 mmol) with a yield of 69.6%.

MS m/z (ESI): 471.2 [M+H]⁺.

### Step 4 N-(3-bromo-2-chlorophenyl)-1-methyl-4,5,6,7-tetrahydro-1H-imidazo[4,5-c]pyridine-2-carboxamide 23e

Compound 23d (500 mg, 1.066 mmol) was dissolved in dichloromethane (10 mL), then added with trifluoroacetic acid (10 mL), and stirred at room temperature for 1 h until the reaction was finished. The reaction solution was concentrated to obtain the title compound 23e (520 mg, 1.066 mmol) with a yield of 100%. The crude product was directly used in the next step.

MS m/z (ESI): 371.1 [M+1]⁺.

### Step 5 N-(3-bromo-2-chlorophenyl)-1-methyl-5-(oxetan-3-yl)-4,5,6,7-tetrahydro-1H-imidazo[4,5-c]pyridine-2-carboxamide 23f

Compound 23e (520 mg, 1.066 mmol) was dissolved in dichloromethane (80 mL), then added with 3-oxetanone (231 mg, 3.21 mmol). Sodium borohydride (680 mg, 3.21 mmol) was added under argon protection in an ice bath, and the temperature was slowly raised to room temperature. The reaction was stirred overnight until it was finished. The reaction solution was quenched with dilute hydrochloric acid, added with saturated sodium bicarbonate, extracted three times with ethyl acetate, washed with saturated brine, dried over anhydrous sodium sulfate, filtered, concentrated and purified by column chromatography to obtain the title compound 23f (360 mg, 0.845 mmol) with a yield of 74%.

MS m/z (ESI): 427.1 [M+H]⁺.

### Step 6 N-(3-(5-amino-4-methylpyridin-3-yl)-2-chlorophenyl)-1-methyl-5-(oxetan-3-yl)-4,5,6,7-tetrahydro-1H-imidazo[4,5-c]pyridine-2-carboxamide 23g

Compound 23f (150 mg, 0.3529 mmol) and 5-amino-4-methylpyridin-3-yl)boronic acid (107 mg, 0.7058 mmol) were dissolved in 1,4-dioxane (3 mL) and water (1 mL), and bis(tri-tert-butylphosphine)palladium (18 mg, 0.03529 mmol) and potassium carbonate (96 mg, 0.7058 mmol) were added, which was heated at 100° C. for 5 h under argon protection until the reaction was finished. The reaction solution was added with water and ethyl acetate for extraction. The organic phase was washed with saturated brine, dried over anhydrous sodium sulfate, filtered, concentrated and purified by column chromatography to obtain the title compound 23g (80 mg, 0.176 mmol) with a yield of 50%.

MS m/z (ESI): 453.3[M+1]⁺

### Step 7 (S)-5-(2-((tert-butyldimethylsilyl)oxy)propyl)-N-(5-(2-chloro-3-(1-methyl-5)-(oxetan-3-yl)-4,5,6,7-tetrahydro-1H-imidazo[4,5-c]pyridine-2-carboxamido)phenyl-4-methylpyridin-3-yl)-1-methyl-4,5,6,7-tetrahydro-1H-imidazo[4,5-c]pyridine-2-carboxamide 23i

Compound 23g (80 mg, 0.176 mmol) and compound 23h (97 mg, 0.265 mmol) were dissolved in toluene (10 mL), and HMDSLi (0.35 mL, 0.35 mmol) was added at room temperature. After the addition, the reaction was continued for 24 h until the reaction was finished. The reaction solution was washed with water, extracted three times with dichloromethane, washed with saturated brine, dried over anhydrous sodium sulfate, filtered, concentrated and purified by column chromatography to obtain the title compound 23i (88 mg, 0.111 mmol) with a yield of 63.3%.

MS m/z (ESI): 788.5 [M+H]⁺.

### Step 8 (S)—N-(5-(2-chloro-3-(1-methyl-5-(oxetan-3-yl)-4,5,6,7-tetrahydro-1H-imidazo[4,5-c]pyridine-2-carboxamido)phenyl)-4-methylpyridin-3-yl)-5-(2-hydroxypropyl)-1-methyl-4,5,6,7-tetrahydro-1H-imidazo[4,5-c]pyridine-2-carboxamide 23

Compound 23i (88 mg, 0.111 mmol) was dissolved in tetrahydrofuran (5 mL), then added with TBAF/THF solution (0.35 mL, 0.35 mmol), stirred at room temperature overnight until the reaction was finished. The reaction solution was added with water, extracted three times with ethyl acetate, washed with saturated brine, dried over anhydrous sodium sulfate, filtered, concentrated and purified by Prep-HPLC to obtain the title compound 23 (25 mg, 0.037 mmol) with a yield of 33%.

MS m/z (ESI): 674.4[M+1]⁺

¹H NMR (400 MHz, DMSO-d₆) δ 10.10 (s, 1H), 9.90 (s, 1H), 8.62 (s, 1H), 8.33 (dd, J=8.3, 1.5 Hz, 1H), 8.18 (s, 1H), 7.47 (t, J=7.9 Hz, 1H), 7.11 (dd, J=7.6, 1.6 Hz, 1H), 4.55 (t, J=6.5 Hz, 2H), 4.46 (t, J=6.1 Hz, 2H), 3.85 (d, J=13.9 Hz, 7H), 3.66 (t, J=6.4 Hz, 2H), 3.19-3.09 (m, 1H), 2.99 (s, 2H), 2.84-2.52 (m, 7H), 1.97 (s, 3H), 1.53 (s, 1H), 1.32-1.21 (m, 1H), 1.05 (d, J=6.1 Hz, 3H), 0.90 (t, J=7.4 Hz, 1H).

### Example 24

### (S)—N-(2-chloro-3-(5-(5-(2-hydroxypropyl)-1-methyl-4,5,6,7-tetrahydro-1H-imidazo[4,5-c]pyridine-2-carboxamido)-4-methylpyridin-3-yl)phenyl)-5-isopropyl-1-methyl-4,5,6,7-tetrahydro-1H-imidazo[4,5-c]pyridine-2-carboxamide

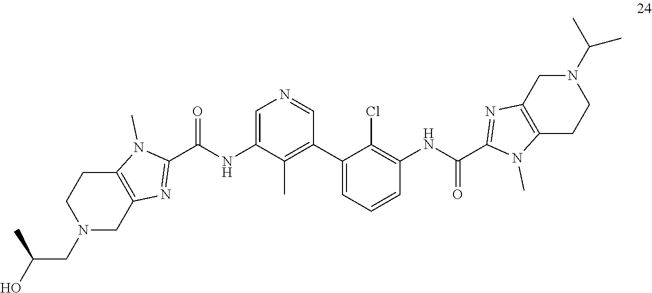 [SMILES: Cc1c(NC(=O)c2nc3c(n2C)CCN(C[C@H](C)O)C3)cncc1-c1cccc(NC(=O)c2nc3c(n2C)CCN(C(C)C)C3)c1Cl]

MS m/z (ESI): 660.5[M+1]⁺

¹H NMR (400 MHz, DMSO-d₆) δ 9.97 (d, J=64.2 Hz, 2H), 8.64 (s, 1H), 8.34 (dd, J=8.3, 1.6 Hz, 1H), 8.17 (s, 1H), 7.47 (t, J=7.9 Hz, 1H), 7.10 (dd, J=7.6, 1.5 Hz, 1H), 4.34 (d, J=4.0 Hz, 1H), 3.84 (d, J=13.5 Hz, 6H), 3.45 (d, J=9.3 Hz, 4H), 2.94-2.86 (m, 1H), 2.76 (dt, J=19.4, 5.7 Hz, 4H), 2.62 (t, J=7.6 Hz, 4H), 2.52 (q, J=1.9 Hz, 1H), 2.43 (d, J=6.9 Hz, 1H), 2.37 (dd, J=12.5, 5.4 Hz, 1H), 1.98 (s, 3H), 1.02 (t, J=6.7 Hz, 9H).

### Example 25

### (R)-1-(((7-cyano-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid

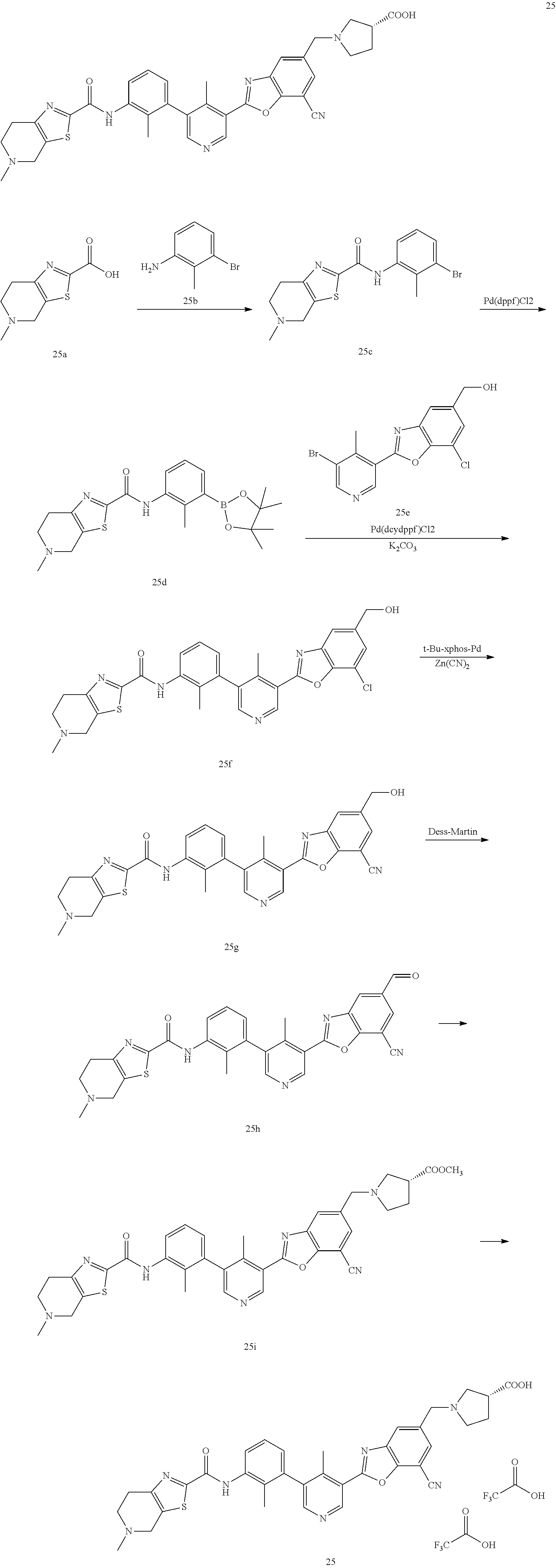

### Step 1 N-(3-bromo-2-methylphenyl)-5-methyl-4,5,6,7-tetrahydrothiazolo[5,4-c]pyridine-2-carboxamide 25c

Compound 25a (3.0 g, 15.13 mmol) and compound 25b (2.82 g, 15.13 mmol) were dissolved in DMF (25 mL), added with HATU (8.62 g, 22.7 mmol) and DIPEA (3.9 g, 30.26 mmol) sequentially, and reacted at room temperature overnight. The reaction solution was diluted with EA (100 mL), washed once with water, washed with saturated brine for several times, dried over anhydrous sodium sulfate, filtered, concentrated and purified by column chromatography to obtain the title compound 25c (2.2 g, 5.33 mmol) as a pale yellow solid with a yield of 35.2%.

MS m/z (ESI): 368.1[M+1]⁺.

### Step 2 5-methyl-N-(2-methyl-3-(4,4,5,5-tetramethyl-1,3,2-dioxaboran-2-yl)phenyl)-4,5,6,7-tetrahydrothiazolo[5,4-c]pyridine-2-carboxamide 25d

Compound 25b (5 g, 13.65 mmol) and diboron pinacol ester (10.4 g, 40.95 mmol) were dissolved in 1,4-dioxane (100 mL). [1,1′-Bis(diphenylphosphino)ferrocene]palladium dichloride (998 mg, 1.365 mmol) and potassium acetate (6.69 g, 68.25 mmol) were added. The reaction was carried out under nitrogen protection, and was heated to 100° C. and stirred for 4 h until the reaction was finished. After cooling to room temperature, water and ethyl acetate were added, and the mixture was stirred for 2 min. The layers were separated. The aqueous phase was extracted with EA. The organic phase was washed with saturated brine, dried over anhydrous sodium sulfate, filtered, concentrated and purified by column chromatography to obtain the title compound 25d (3 g, 7.26 mmol) with a yield of 54.8%.

MS m/z (ESI): 414.3[M+1]⁺.

### Step 3 N-(3-(5-(7-chloro-5-(hydroxymethyl)benzo[d]oxazol-2-yl]-4-methylpyridin-3-yl)-2-methylphenyl)-5-methyl-4,5,6,7-tetrahydrothiazolo[5,4-c]pyridine-2-carboxamide 25f

Compound 25d (1 g, 2.42 mmol), compound 25e (854 mg, 2.42 mmol), Pd(dcydppf)Cl₂ (177 mg, 0.242 mmol) and K₂CO₃ (1 g, 7.26 mmol) were dissolved in dioxane (30 mL) and H₂O (6 mL), heated to 90° C. under argon protection and stirred for 3 h. After cooling to room temperature, the layers were separated. The aqueous phase was extracted with ethyl acetate. The organic phases were combined, washed once with saturated brine, dried over anhydrous sodium sulfate, filtered, concentrated and purified by column chromatography to obtain the title compound 25f (500 mg, 0.892 mmol) as white solid with a yield of 36.9%.

MS m/z (ESI): 560.3[M+1]⁺.

### Step 3 N-(3-(5-(7-cyano-5-(hydroxymethyl)benzo[d]oxazol-2-yl)-4-methylpyridin-3-yl)-2-methylphenyl)-5-methyl-4,5,6,7-tetrahydrothiazolo[5,4-c]pyridine-2-carboxamide 25g

Compound 25f (500 mg, 0.893 mmol), Zn(CN)₂ (209 mg, 1.785 mmol) and t-Bu-X-phos-PdCl₂ (141 mg, 0.179 mmol) were added to a mixed solvent of dioxane (25 mL)/H₂O (25 mL), heated to 90° C. under argon protection and stirred for 4 h.

After cooling to room temperature, the layers were separated. The aqueous phase was extracted with ethyl acetate. The organic phases were combined, washed once with saturated brine, dried over anhydrous sodium sulfate, filtered, concentrated and purified by column chromatography to obtain the title compound 25g (350 mg, 0.892 mmol) as white solid with a yield of 63.6%.

MS m/z (ESI): 551.3[M+1]⁺.

### Step 5 N-(3-(5-(7-cyano-5-formylbenzo[d]oxazol-2-yl)-4-methylpyridin-3-yl)-2-methylphenyl)-5-methyl-4,5,6,7-tetrahydrothiazolo[5,4-c]pyridine-2-carboxamide 25h

Compound 25g (380 mg, 0.690 mmol) was dissolved in DCM (25 mL), and added with DMP (380 mg, 0.897 mmol) at room temperature with continued stirring for 1 h. The end of the reaction was determined by LCMS detection. The reaction was quenched by adding sodium thiosulfate aqueous solution and sodium bicarbonate aqueous solution. The layers were separated. The aqueous phase was extracted with DCM. The organic phases were combined, dried over anhydrous sodium sulfate, filtered, concentrated and purified by column chromatography to obtain the title compound 25h (300 mg, 0.655 mmol) with a yield of 94.5%.

MS m/z (ESI): 549.3[M+1]⁺.

### Step 6 methyl (R)-1-(((7-cyano-2-(4-methyl-5-(2-methyl-3-(5-methyl-4,5,6,7-tetrahydrothiazole)[5,4-c]pyridine-2-carboxamido)phenyl)pyridin-3-yl)benzo[d]oxazol-5-yl)methyl)pyrrolidine-3-carboxylate 25i

Compound 25h (150 mg, 0.273 mmol) and methyl (R)-pyrrolidine-3-carboxylate hydrochloride (91 mg, 0.547 mmol) were dissolved in dichloromethane (20 mL). The reaction was stirred at room temperature for 30 minutes. After that, sodium borohydride acetate (289 mg, 1.367 mmol) was added, and stirred at room temperature overnight until the reaction was finished as determined by LCMS detection. The reaction solution was quenched with water, and extracted three times with dichloromethane. The organic phases were combined, washed with saturated brine, dried over anhydrous sodium sulfate, filtered, concentrated and purified by column chromatography to obtain the title compound 25i (120 mg, 0.181 mmol) as a yellow foamy solid with a yield of 66.4%.

MS m/z (ESI): 662.5[M+1]⁺.

### Step 7 (R)-1-(((7-cyano-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid

Compound 25i (100 mg, 0.151 mmol) was added to a mixed solvent of MeCN (4 mL)/H₂O (4 mL), and added with LiOH (4 mg, 0.167 mmol) under stirring. The mixture was stirred at room temperature for 2 h. After the reaction was complete as determined by LCMS detection, the pH of the reaction solution was adjusted to neutral with dilute HCl, and separated and purified by pre-HPLC to obtain the target compound 25 (65 mg, 0.0742 mmol) with a yield of 49.2%.

MS m/z (ESI):648.5 [M+1]⁺

¹H NMR (400 MHz, DMSO-d₆) δ 10.49 (s, 3H), 9.25 (s, 1H), 8.53 (s, 111), 8.40 (d, J=1.5 Hz, 1H), 8.14 (d, J=1.5 Hz, J H), 7.52 (dd, J=8.1, 1.3 Hz, 1H), 7.37 (t. J=7.8 Hz, 1H), 7.16 (dd, J=7.6, 1.3 Hz, 1H), 4.67 (d, J=79.8 Hz, 4H), 3.79-3.10 (m, 9H), 2.97 (s, 3H), 2.45 (d, J=1.8 Hz, 3H), 2.38-2.12 (m, 2H), 1.94 (s, 3H).

### Example 26

### N-(5-(2-chloro-3-(5-((S)-2-hydroxypropyl)-1-methyl-4,5,6,7-tetrahydro-1H-imidazo[4,5-c]pyridine-2-carboxamido)phenyl)-4-methylpyridin-3-yl)-5-((S)-2-hydroxypropyl)-1-methyl-4,5,6,7-tetrahydro-1H-imidazo[4,5-c]pyridine-2-carboxamide

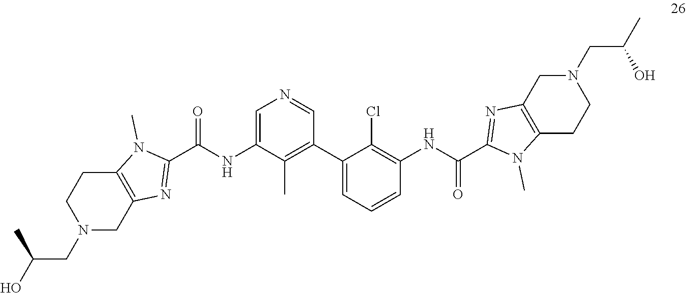 [SMILES: Cc1c(NC(=O)c2nc3c(n2C)CCN(C[C@H](C)O)C3)cncc1-c1cccc(NC(=O)c2nc3c(n2C)CCN(C[C@H](C)O)C3)c1Cl]

A synthetic method similar to that of Example 23 was used to prepare the title product 26.

MS m/z (ESI):676.5[M+1]⁺

¹H NMR (400 MHz, Methanol-d₄) δ 9.52 (s, 1H), 8.65 (s, 1H), 8.47 (d, J=8.2 Hz, 1H), 7.58 (t, J=7.9 Hz, 1H), 7.31 (d, =7.4 Hz, 1H), 4.58 (s, 2H). 4.35 (d, J=49.6 Hz, 4H), 4.03 (d, J=1.9 Hz, 8H), 3.65 (s, 2H), 3.39 (s, 2H), 3.20 (d, J=26.1 Hz, 6H), 2.44 (s, 3H), 1.25 (dd, J=6.1, 1.9 Hz, 6H).

### Example 27

### N-(2-chloro-3-(5-(5-ethyl-1-methyl-4,5,6,7-tetrahydro-1H-imidazo[4,5-c]pyridine-2-carboxamido)-4-methylpyridin-3-yl)phenyl)-5-ethyl-1-methyl-4,5,6,7-tetrahydro-1H-imidazo[4,5-c]pyridine-2-carboxamide

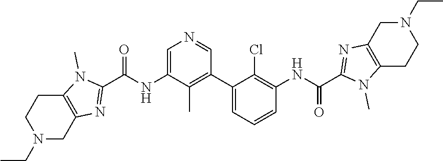 [SMILES: CCN1CCc2c(nc(C(=O)Nc3cncc(-c4cccc(NC(=O)c5nc6c(n5C)CCN(CC)C6)c4Cl)c3C)n2C)C1]

A synthetic method similar to that of Example 23 was used to prepare the title product 27.

MS m/z (ESI): 616.5[M+1]⁺

¹H NMR (400 MHz, DMSO-d₆) δ 10.25 (s, 3H), 9.94 (s, 1H), 8.64 (s, 1H), 8.31-8.17 (m, 2H), 7.50 (t, J=7.9 Hz, 1H), 7.16 (dd, J=7.6, 1.6 Hz, 1H), 4.41 (d, J=14.7 Hz, 2H), 4.20 (s, 2H), 3.90 (d, J=16.6 Hz, 6H), 3.79 (s, 2H), 3.39 (s, 2H), 3.28 (s, 4H), 2.98 (d, J=15.2 Hz, 4H), 1.99 (s, 3H), 1.28 (td, J=7.2, 4.1 Hz, 6H).

### Example 28

### (R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid

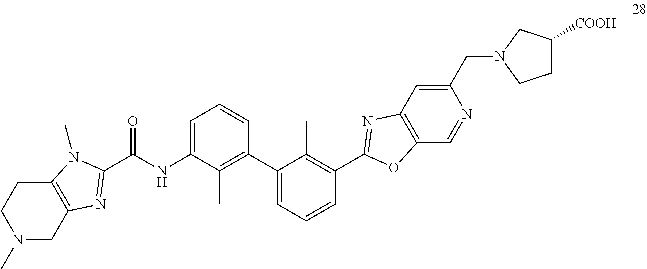 [SMILES: Cc1c(NC(=O)c2nc3c(n2C)CCN(C)C3)cccc1-c1cccc(-c2nc3cc(CN4CC[C@@H](C(=O)O)C4)ncc3o2)c1C]

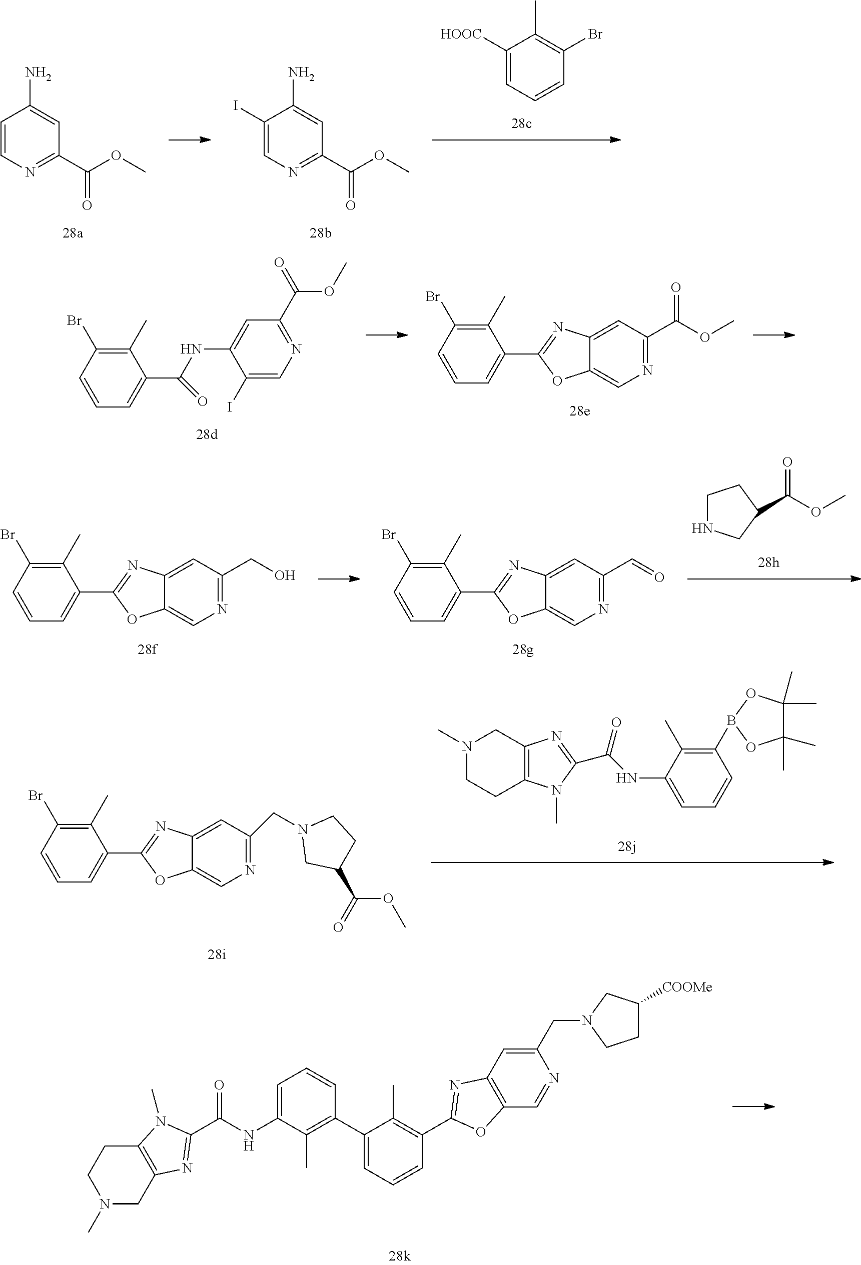

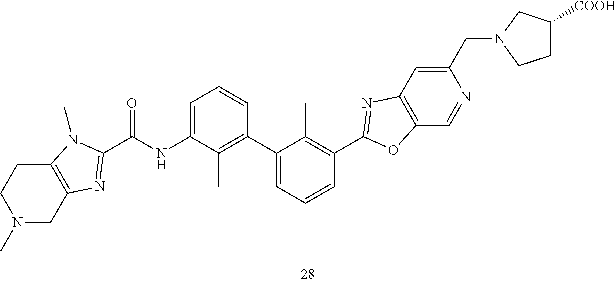 [SMILES: Cc1c(NC(=O)c2nc3c(n2C)CCN(C)C3)cccc1-c1cccc(-c2nc3cc(CN4CC[C@@H](C(=O)O)C4)ncc3o2)c1C]

### Step 1 methyl 4-amino-5-iodopicolinate

Compound 28a (25 g, 0.16 mol) and NIS (44 g, 0.19 mol) were dissolved in DCM (200 mL) and heated under reflux for 8 h. The reaction solution was added with water and filtered. The filter cake was washed twice with DCM. The combined filtrate were washed with saturated brine for several times, concentrated and purified by column chromatography to obtain the title compound 28b (18 g, yield 40%).

MS m/z (ESI): 278[M+1]⁺.

### Step 2 methyl 4-(3-bromo-2-methylbenzamido)-5-iodopicolinate

Compound 28c (28 g, 0.13 mol) was dissolved in dichloromethane (300 mL), and oxalyl chloride (68 g, 0.54 mol) was added under ice bath. The reaction was stirred at room temperature for 2 h. The reaction solution was evaporated until dried, and dissolved by adding 100 ml dichloromethane for later use. In another three-necked flask, compound 28b (30 g, 0.11 mol) was dissolved in dichloromethane (300 mL), added with DIPEA (56 g, 0.43 mol), and added dropwise with the above-mentioned solution for later use. The mixture was stirred at room temperature overnight. Water was added to the above solution under stirring. The layers was separated and the organic phase was concentrated, and went through a flash column to obtain the title compound 28d (25 g, yield 48%).

MS m/z (ESI): 477[M+1]⁺.

### Step 3 methyl 2-(3-bromo-2-methylphenyl)oxazolo[5,4-c]pyridine-6-carboxylate

Compound 28d (1.0 g, 2.1 mmol), phenanthroline (72 mg, 0.4 mmol), CuI (76 mg, 0.4 mmol) and Cs₂CO₃ (1.4 g, 4.2 mmol) were added to dioxane (10 mL) and stirred at 110° C. for 16 h. The resultant was subjected to suction filtration. The filter cake was pulped with DCM/MeOH, and subjected to suction filtration. The filtrate was combined, concentrated and purified by column chromatography to obtain compound 28e (2 g, yield 13%).

MS m/z (ESI): 347[M+1]⁺.

### Step 4 (2-(3-bromo-2-methylphenyl)oxazolo[5,4-c]pyridin-6-yl)methanol

Compound 28e (2 g, 5.7 mmol) was dissolved in DCM/THF (10 mL), cooled to −70° C., and added with DIBAL-H (11 mL, 11.4 mmol), which was naturally warmed to room temperature and stirred for 1 h, and then added with sodium potassium tartrate aqueous solution under stirred until the layers were separated.

The organic phase was concentrated and passed through a flash column to obtain 28f (1.8 g, 90% yield) as a white solid.

MS m/z (ESI): 321[M+1]⁺.

### Step 5 2-(3-bromo-2-methylphenyl)oxazolo[5,4-c]pyridine-6-formaldehyde

Compound 28f (2 g, 6.26 mmol) was dissolved in dichloromethane (50 mL), added with DMP (4 g, 9.40 mmol), and reacted at room temperature for 2 h. The reaction solution was added with Na₂SO₃ aqueous solution and NaHCO₃ aqueous solution, and was stirred until the layers were separated. The organic phase was dried over anhydrous sodium sulfate, concentrated and purified by column chromatography to obtain compound 28g (1.8 g, yield 90%).

MS m/z (ESI): 320.9[M+1]⁺.

### Step 6 methyl (R)-1-(((2-(3-bromo-2-methylphenyl)oxazolo[5,4-c]pyridin-6-yl)methyl)pyrrolidine-3-carboxylate

Compound 28g (2 g, 6.3 mmol) and compound 28h (1.6 g, 12.6 mmol) were dissolved in dichloromethane (50 mL) and methanol (2 mL), added with sodium acetylborohydride (1.3 g, 6.3 mmol), and stirred overnight at room temperature until the reaction was finished. The reaction solution was quenched with NaHCO₃ aqueous solution, and extracted three times with dichloromethane. The combined organic phases were dried over anhydrous sodium sulfate, filtered, concentrated and purified by column chromatography to obtain the title compound 28i (1 g, 36%).

MS m/z (ESI): 430[M+1]⁺.

### Step 7 methyl (R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylate

Compound 28i (251 mg, 0.58 mmol) and compound 28j (240 mg, 0.58 mmol), Pd(dppf)Cl₂ (84 mg, 0.12 mmol), K₂CO₃ (165 mg, 1.2 mmol) were dissolved in dioxane (20 mL) and water (2 mL). The atmosphere was replaced with N₂. The reaction carried out at 110° C. for 4 h. The reaction solution was added with water, and extracted three times with dichloromethane. The organic phases were combined, dried over anhydrous sodium sulfate, filtered, concentrated and purified by column chromatography to obtain the title compound 28k (150 mg, 40%), which was directly used in the next step.

MS m/z (ESI): 634.4[M+1]⁺.

### Step 8 (R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid

Compound 28k (150 mg, 0.24 mmol) was dissolved in acetonitrile (5 mL), and water (2 mL) was added. The mixture was stirred at room temperature for 2 h. The pH value was adjusted to weakly acidic with acetic acid. The title compound 28 (120 mg, yield 80%) was obtained by preparative purification.

MS m/z (ESI): 620.5[M+1]⁺

¹H NMR (400 MHz, DMSO-d₆) δ 10.59 (s, 2H), 9.95 (s, 1H), 9.24 (d, J=1.0 Hz, 1H), 8.17 (dd, J=7.9, 1.4 Hz, 1H), 8.05 (d, J=1.0 Hz, 1H), 7.60-7.49 (m, 2H), 7.40 (dd. J=7.7, 1.5 Hz, 1H), 7.30 (t, J=7.8 Hz, 1H), 7.02 (dd, J=7.7, 1.4 Hz, 1H), 4.66 (s, 2H), 4.41 (d, J=11.4 Hz, 1H), 4.18 (s, 1H), 3.88 (s, 3H), 3.72 (d, J=11.0 Hz, 1H), 3.42 (d, J=99.0 Hz, 6H), 2.96 (d, J=18.3 Hz, 6H), 2.41 (s, 3H), 2.21 (d, J=51.9 Hz, 2H), 1.92 (s, 3H).

### Example 29

### (R)-1-((2-(3′-(5-(2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid

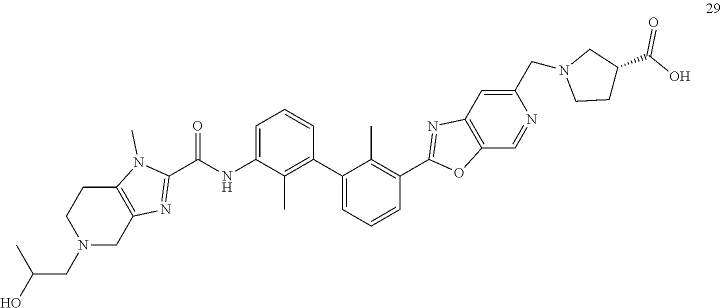 [SMILES: Cc1c(NC(=O)c2nc3c(n2C)CCN(CC(C)O)C3)cccc1-c1cccc(-c2nc3cc(CN4CC[C@@H](C(=O)O)C4)ncc3o2)c1C]

A synthetic method similar to that of Example 28 was used to prepare the title product 29.

MS m/z (ESI): 664.5[M+1]⁺

¹H NMR (400 MHz, DMSO-d₆) δ 10.58 (s, 1H), 10.11 (s, 1H), 9.91 (s, 1H), 9.24 (d, J=1.0 Hz, 1H), 8.17 (dd, J=7.9, 1.5 Hz, 1H), 8.05 (d, J=1.0 Hz, 1H), 7.59 (d, J=8.0 Hz, 1H), 7.54 (t, J=7.7 Hz, 1H), 7.40 (dd, J=7.7, 1.5 Hz, 1H), 7.30 (t, J=7.8 Hz, 1H), 7.02 (dd, J=7.6, 1.4 Hz, 1H), 4.66 (s, 2H), 4.33 (dd, J=55.6, 15.0 Hz, 5H), 4.13 (d, J=9.2 Hz, 2H), 3.88 (s, 3H), 3.26 (s, 3H), 3.11-2.96 (m, 3H), 2.41 (s, 3H), 2.21 (d, J=51.1 Hz, 2H), 1.92 (s, 3H), 1.10 (d, J=6.1 Hz, 3H).

### Example 30

### (R)-1-(((7-chloro-2-(3-(5-(5-((S)-2-hydroxypropyl))-1-methyl-4,5,6,7-tetrahydro-1H-imidazo[4,5-c]pyridine-2-carboxamido)-4-methylpyridin-3-yl)-2-methylphenyl)benzo[d]oxazole-5-yl)methyl)pyrrolidine-3-carboxylic acid

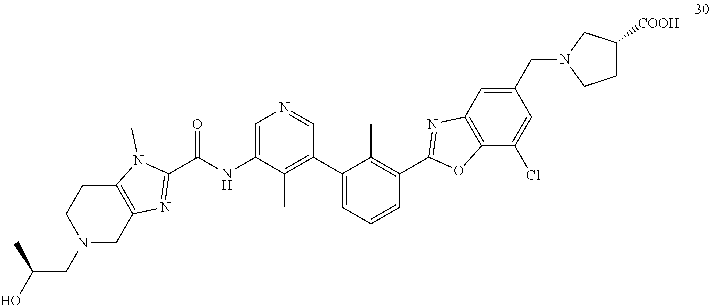 [SMILES: Cc1c(NC(=O)c2nc3c(n2C)CCN(C[C@H](C)O)C3)cncc1-c1cccc(-c2nc3cc(CN4CC[C@@H](C(=O)O)C4)cc(Cl)c3o2)c1C]

A synthetic method similar to that of Example 8 was used to prepare the title product 30.

MS m/z (ESI): 698[M+]⁺

¹H NMR (400 MHz, DMSO-d₆+D2O) δ 10.94 (s, 1H), 10.52 (s, 1H), 10.35 (dd, J=8.0, 1.4 Hz, 1H), 10.15 (d, J=1.4 Hz, 1H), 9.90 (d, J=1.4 Hz, 1H), 9.76 (t, J=7.8 Hz, 1H), 9.63 (dd, J=7.7, 1.4 Hz, 1H), 6.56 (d, J=87.5 Hz, 4H), 6.29 (ddd, J=9.9, 6.4, 2.8 Hz, 1H), 6.03 (s, 3H), 5.68 (d, J=53.8 Hz, 5H), 5.41 (d, J=14.5 Hz, 2H), 5.31-5.06 (m, 4H), 4.56 (s, 3H), 4.38 (d, J=12.8 Hz, 2H), 4.26-4.18 (m, 3H), 3.28 (d, J=6.1 Hz, 3H).

Biological Activity Test

Experiment 1. Determination of the In Vitro Activity of the Small Molecule Compound of the Invention on Human Programmed Death Receptor Ligand (PD-L1)

1. Aim of the Experiment

In this experiment, Homogeneous Time-Resolved Fluorescence (HTRF) was used to test the blocking effect of the compound of the present invention on the binding between human programmed death receptor (PD-1) and human programmed death receptor ligand (PD-L1), in which the in vitro activity of the compound was evaluated according to the half inhibitory concentration (IC50).

2. Experimental Method

2.1 Configuration of Experimental Compounds

Compounds 1-30 and BMS202 used in this experiment were dissolved in dimethyl sulfoxide (DMSO) to prepare a stock solution of 10 millimoles per liter (mM). The highest concentration of test compound was 0.1 micromolar per liter (μM), which was diluted 10-fold, with a total of 6 concentration gradients, each repeated in double wells.

2.2 Experimental Process

According to the instruction manual of Human Programmed Death Receptor (PD-1) and Human Programmed Death Receptor Ligand (PD-L1) Binding Detection Kit (Cat. No.: 64ICP01PEG) from Cisbio, 2 microliters (μL) serially diluted compound, 4 microliters (L) human programmed death receptor (PD-1) protein and 4 microliters (μL) human programmed death receptor ligand (PD-L1) protein were added to 384 wells, respectively, and reacted at room temperature for 15 min. After that, 5 microliters (μL) of lanthanide europium (Eu) labeled anti-human immunoglobulin Fc fusion protein antibody (anti-hFc) and 5 microliters (μL) of modified allophycocyanin (XL665) labeled anti-histidine antibody (anti-His) were added, respectively. The total reaction system was 20 microliters (μL). The reaction was performed at room temperature for 2 h. Finally, the 665 nanometer (nm) and 620 nanometer (nm) fluorescence values were read on a microplate reader. The Homogeneous Time-Resolved Fluorescence (HTRF) rate was (665 nm)/(620 nm)×104. The half inhibitory concentration (IC50) value was calculated using Graphpad Prism 7.0 software.

2.3 Experimental Results

The compound of the present invention has an obvious inhibitory effect on the binding between human programmed death receptor (PD-1) and human programmed death receptor ligand (PD-L1), and the detected half-inhibitory concentration (IC50) of the in vitro activity is less than 10 nM for each compound. The IC50 value of the positive drug BMS202 was 16.61 nanomoles per liter (nM).

Conclusion: The compound of the present invention exhibited significant inhibitory activity on the binding between human programmed death receptor (PD-1) and human programmed death receptor ligand (PD-L1), and the inhibitory activity is significantly higher than that of BMS202.

Experiment II. Pharmacokinetic Experiment of the Compound of the Invention in Rats

1. Aim of the Experiment:

Taking SD rats as an example, the drug concentrations in the plasma of rats at different times after i.g. and i.v. administration of compounds of Examples 1, 5, 6, 8, 12, 13, 15, 18, 19, 22, 25, 27 and 29, respectively, were determined by the liquid chromatography-tandem mass spectrometry (LS/MS/MS) method, and the relevant pharmacokinetic parameters were calculated to evaluate the pharmacokinetic properties of example compounds of the present invention in rats.

2. Test Protocol

2.1 Test Drug

Compounds of Example 1, Example 5, Example 6, Example 8, Example 12, Example 13, Example 15, Example 18, Example 19, Example 22, Example 25, Example 27, and Example 29.

2.2 Test Animal

Healthy adult SD rats, SPF grade, male, purchased from Sippe-Bk Lab Animal Co., Ltd., Shanghai, license number SCXK (Shanghai) 2018-0006, animal certificate number: 20180006006321.

2.3 Drug Formulation

A certain amount of compound was weighed, dissolved in 5% dimethyl sulfoxide (DMSO), subjected to vortex and sonication, added with 0.5% sodium carboxymethyl cellulose (CMC-Na), and sonicated, to prepare a homogeneous solution.

2.4 Dosing

The specific dosing was carried out according to the test protocol, and the details are shown in Table 2.

TABLE 2
Pharmacokinetic Test Protocol in Rats

| | Number | | | | Admin- |
|---|---|---|---|---|---|
| | of | | Concentration | Dosage | istration |
| Group | animals | Test drug | (mg/kg) | (mL/kg) | route |
| 1 | 4 | Example 1 | 10 | 10 | i.g. |
| | | compound | 2 | 10 | i.v. |
| 2 | 4 | Example 5 | 10 | 10 | i.g. |
| | | compound | 2 | 10 | i.v. |
| 3 | 4 | Example 6 | 10 | 10 | i.g. |
| | | compound | 2 | 10 | i.v. |
| 4 | 4 | Example 8 | 10 | 10 | i.g. |
| | | compound | 2 | 10 | i.v. |
| 5 | 4 | Example 12 | 10 | 10 | i.g. |
| | | compound | 2 | 10 | i.v. |
| 6 | 4 | Example 13 | 10 | 10 | i.g. |
| | | compound | 2 | 10 | i.v. |
| 7 | 4 | Example 15 | 10 | 10 | i.g. |
| | | compound | 2 | 10 | i.v. |
| 8 | 4 | Example 18 | 10 | 10 | i.g. |
| | | compound | 2 | 10 | i.v. |
| 9 | 4 | Example 19 | 10 | 10 | i.g. |
| | | compound | 2 | 10 | i.v. |
| 10 | 4 | Example 22 | 10 | 10 | i.g. |
| | | compound | 2 | 10 | i.v. |
| 11 | 4 | Example 25 | 10 | 10 | i.g. |
| | | compound | 2 | 10 | i.v. |
| 12 | 4 | Example 27 | 10 | 10 | i.g. |
| | | compound | 2 | 10 | i.v. |
| 13 | 4 | Example 29 | 10 | 10 | i.g. |
| | | compound | 2 | 10 | i.v. |

2.5 Blood Sampling Schedule and Sample Processing

Before administration and 5 min, 15 min, 0.5 h, 1 h, 2 h, 4 h, 6 h, 8 h and 24 h after administration, orbital blood was collected, 0.2 milliliters (mL) of blood being collected each time, and placed in an anticoagulant tube, mixed well, and stored in a −20° C. refrigerator for later use.

3. Sample Testing and Data Analysis

The content of the compound in rat whole blood was determined by liquid chromatography-tandem mass spectrometry (LS/MS/MS).

The pharmacokinetic parameters of the compounds after administration were calculated using the non-compartmental model of WinNonLin5.3 software.

4. Test Results

The pharmacokinetic parameters of the compounds of the present invention after administration are shown in Table 3 below. As shown in Table 3, the compounds of the present invention have better metabolic characteristics and bioavailability.

TABLE 3
Pharmacokinetic parameters of compounds of the invention

| | i.v.-Dosage (2 mg/kg) | | p.o.-Dosage (10 mg/kg ) | | |
|---|---|---|---|---|---|
| | Area under | | Blood | Area under | |
| | the curve | | concentration | the curve | Bio- |
| Example | (AUC, | Half life | (Cmax, | (AUC, | availability |
| compound | ng/mL.h) | (T1/2, h) | ng/mL) | ng/mL.h) | (F%) |
| 1 | 595.57 | 2.05 | 415.00 | 1667.6 | 56 |
| 5 | 611.5 | 2.5 | 432.12 | 2079.1 | 68 |
| 6 | 675.9 | 1.36 | 389.15 | 2095.29 | 62 |
| 8 | 601.18 | 1.56 | 402.12 | 1654.64 | 55 |
| 12 | 585.87 | 2 22 | 441.51 | 1757.62 | 60 |
| 13 | 629.62 | 1.43 | 431.2 | 1699.97 | 54 |
| 15 | 599.36 | 1.76 | 377.6 | 1618.28 | 58 |
| 18 | 578.3 | 2.42 | 365.4 | 1532.5 | 53 |
| 19 | 560.44 | 2.21 | 406.78 | 1821.44 | 65 |
| 22 | 577.95 | 1.46 | 488.2 | 1473.78 | 51 |
| 25 | 611.79 | 2.35 | 435.02 | 1835.37 | 60 |
| 27 | 636.52 | 2.26 | 423.17 | 1463.98 | 46 |
| 29 | 617.33 | 2.14 | 405.10 | 1481.59 | 48 |

Experiment 3. In Vivo Efficacy Experiment of the Compound of the Present Invention in Mice

1. Dosing Regimen

Under sterile conditions, mouse colon cancer MC38-hPD-L1 in logarithmic growth phase was digested into single cells and then transplanted into the right back of C57BL/6 mice subcutaneously. Each mouse was inoculated with 1×10⁶ cells in a volume of 100 liter (μL). After inoculation, when the tumor grows to 100 cubic millimeters (mm³), the mice were randomly divided into 9 groups, with 8 mice in each group, for drug efficacy experiments. The positive control was human programmed death receptor ligand (PD-L1) antibody. The negative control group was given the same amount of vehicle. The specific design is shown in Table 4.

TABLE 4
Experimental Protocol for In Vivo Pharmacodynamics of Compounds

| | | | | Administration | | |
|---|---|---|---|---|---|---|
| | Number of | | Dosage | volume | Administration | Schedule of |
| Group | animals | Test drug | (mg/kg) | (mL/kg) | route | administration |
| 1 | 8 | Vehicle | NA | 10 | i.g. | Twice a day, 21 times in total |
| 2 | 8 | PD-L1 antibody | 5 | 10 | i.p. | Twice a week, 21 days in total |
| 3 | 8 | Example compound 4 | 50 | 10 | i.g. | Twice a day, 21 times in total |
| 4 | 8 | Example compound 9 | 50 | 10 | i.g. | Twice a day, 21 times in total |
| 5 | 8 | Example compound 11 | 50 | 10 | i.g. | Twice a day, 21 times in total |
| 6 | 8 | Example compound 15 | 50 | 10 | i.g. | Twice a day, 21 times in total |
| 7 | 8 | Example compound 23 | 50 | 10 | i.g. | Twice a day, 21 times in total |
| 8 | 8 | Example compound 27 | 50 | 10 | i.g. | Twice a day, 21 times in total |
| 9 | 8 | Example compound 30 | 50 | 10 | i.g. | Twice a day, 21 times in total |

2. Test Animal

C57BL/6 mice, SPF grade, 6-8 weeks old, female, purchased from Beijing Vital River Laboratory Animal Technology Co., Ltd., license number SCXK (Shanghai) 2017-0011, animal certificate number: 20170011003865.

3. Test Drug

Compounds of Example 4, Example 9, Example 11, Example 15, Example 23, Example 27, Example 30.

4. Drug Formulation

An appropriate amount of compound was weighted, dissolved in 5% dimethyl sulfoxide (DMSO), subjected to vortex and sonication, added with 0.5% sodium carboxymethyl cellulose (CMC-Na), and sonicated, to prepare a solution of 5 mg per milliliter (mg/mL).

5. Dosing

The specific dosing was carried out according to the Dosing regimen.

6. Experimental Results and Conclusions

Tumor growth inhibition rate of compounds of Example 4, Example 9, Example 11, Example 15, Example 23, Example 27, and Example 30 at an administration concentration of 50 milligrams per kilogram (mg/kg) is 82%, 78%, 85%, 86%, 81%, 80%, and 79%, respectively, while the tumor growth inhibition rate of PD-L1 antibody at an administration concentration of 5 milligrams per kilogram (mg/kg) was only 65%. This indicates that compounds of Example 4, Example 9, Example 11, Example 15, Example 23, Example 27, and Example 30 of the present invention showed better tumor growth inhibition effect in the mouse colon cancer MC38-hPD-L1 model than the control human programmed death ligand (PD-L1) antibody.

Experiment 4. Acute Toxicity Test of the Compound of the Present Invention in Mice

1. Aim of the Experiment

This experiment aims to test the toxic effects of the compounds in mice.

2. Test Animal

ICR mice, SPF grade, 6-8 weeks old, half male and half male, purchased from Sippe-Bk Lab Animal Co., Ltd., Shanghai, license number SCXK (Shanghai) 2018-0006, animal certificate number: 20180006010983.

3. Test Drug

Compounds of Example 2, Example 4, Example 9, Example 11, Example 13, Example 19, Example 21, Example 24, Example 26, and Example 30.

4. Experimental Method

ICR mice were administered different doses of compounds at a single time, and were continuously observed for 14 days. The animal death, poisoning reaction, body weight change, diet, appearance, behavior, etc. were recorded. At the end point, the animals were dissected, and the organs were taken for histopathological examination.

5. Experimental Results and Conclusions

The median lethal doses (LD50) of compounds of Example 2, Example 4, Example 9, Example 11, Example 13, Example 19, Example 21, Example 24, Example 26 and Example 30 are greater than 1000 milligrams per kilogram (mg/kg). Compared with the mice in the control group, the mice in the administration group have no abnormal body weight or behavior within 14 days from the date of administration, which showed that compounds of Example 2, Example 4, Example 9, Example 11, Example 13, Example 19, Example 21, Example 24, Example 26 and Example 30 are very safe.

The above are only preferred embodiments of the present invention and are not intended to limit the present invention. Any modification, equivalent replacement, improvement, etc. made within the spirit and principle of the present invention shall be included in the protection scope of the present invention.

1. A compound represented by general formula (I), or a tautomer, mesomer, racemate, enantiomer, diastereomer, mixture of isomers, pharmaceutically acceptable salt, polymorph, solvate, prodrug, metabolite, or isotopic derivative thereof, 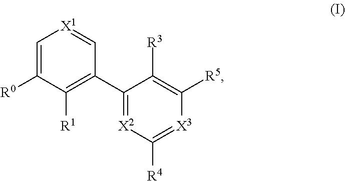
wherein
X¹ is C—R², X² is C, and X³ is C;
X¹ is N, X² is C, and X³ is C;
X¹ is C—R², X² is C, and X³ is N;
X¹ is C—R², X² is N, and X³ is C; or
X¹ is C—R², X² is N, and X³ is N; and
R⁰ is —NR^{a}R⁶ or —NR^{a}C(O)R⁶;
R¹, R³ and R⁴ are each independently hydrogen, halogen or C₁₋₆ alkyl;
R² is hydrogen or C₁₋₆ alkyl;
R⁵ is C₅₋₁₂ fused heterobicyclic group, —NR^{b}R⁷ or —NR^{b}C(O)R⁷, wherein optionally one or more hydrogen atom of the C₅₋₁₂ fused heterobicyclic group is independently substituted by R^{d};
R⁶ is C₃₋₇ heterocycloalkyl, C₃₋₉ heteroaryl, C₆₋₁₂ fused bicyclic group or C₅₋₁₂ fused heterobicyclic group, wherein optionally one or more hydrogen atom on R⁶ is independently substituted by R^{e};
R⁷ is C₃₋₇ heterocycloalkyl, C₃₋₉ heteroaryl, C₆₋₁₂ fused bicyclic group, C₅₋₁₂ fused heterobicyclic group, wherein optionally one or more hydrogen atom on R⁷ is independently substituted by R^{f};
R^{a} and R^{b} are each independently hydrogen, —CH₃ or —CH(CH₃)₂;
R^{d} is independently halogen, cyano, C₁₋₆ alkyl, C₃₋₇ heterocycloalkyl or -methylene-C₃₋₇ heterocycloalkyl, wherein said C₁₋₆ alkyl, C₃₋₇ heterocycloalkyl, or -methylene-C₃₋₇ heterocycloalkyl is optionally substituted by —CH₃, —OH, —COOH, or —COOCH₃;
R^{e} and R^{f} are each independently hydrogen, C₁₋₆ alkyl, —(CH₂)ₙ—OH, —(CH₂)ₙ—NH₂, —(CH₂)ₙ—NH—(CH₂)ₙ—CH₃, —O—CH₃, C₃₋₆ cycloalkyl, C₃₋₇ heterocycloalkyl, -methylene-C₃₋₇ heterocycloalkyl or C₆₋₁₀ aryl, wherein said C₁₋₆ alkyl, —(CH₂)ₙ—OH, —(CH₂)ₙ—NH₂, —(CH₂)ₙ—NH—(CH₂)ₙ—CH₃, C₃₋₆ cycloalkyl, C₃₋₇ heterocycloalkyl, -methylene-C₃₋₇ heterocycloalkyl, or C₆₋₁₀ aryl is optionally substituted by R^{j};
R^{j} is halogen, hydroxyl, —NH₂, C₁₋₆ alkyl, —COOH, —COOCH₃, —(CH₂)ₙ—OH, —(CH₂)ₙ—NH—CH₃, —(CH₂)ₙ—O—CH₃ or —(CH₂)ₙ—NH—C(O)—CH₃; and
n is 0, 1 or 2.

2. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
R⁰ is —NR^{a}R⁶ or —NR^{a}—C(O)—R⁶;
R¹, R³ and R⁴ are each independently —H, —F, —Cl or —CH₃;
R⁵ is —NR^{b}R⁷, —NR^{b}—C(O)—R⁷, 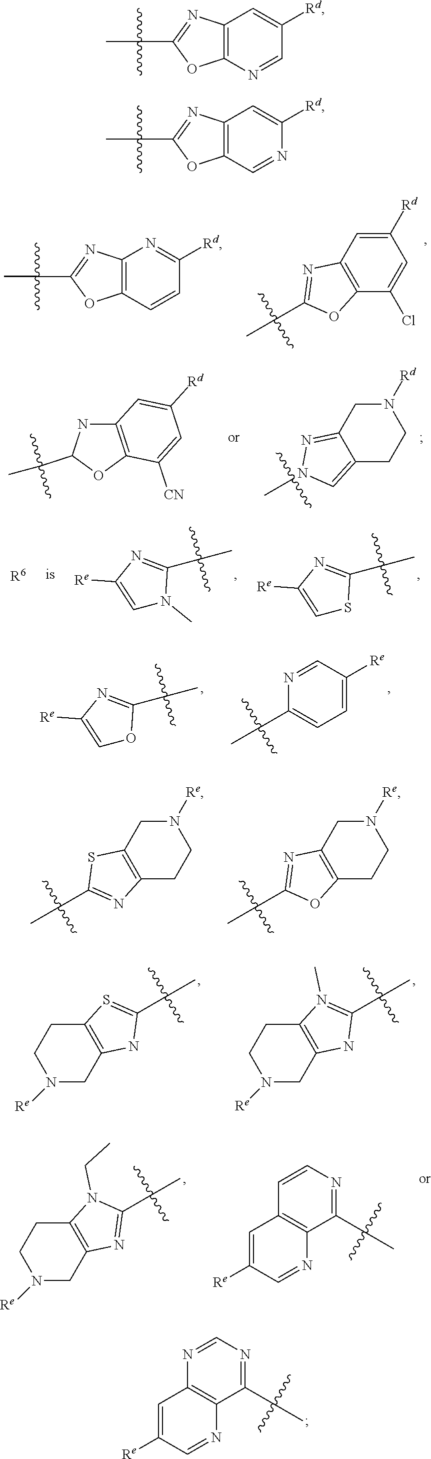 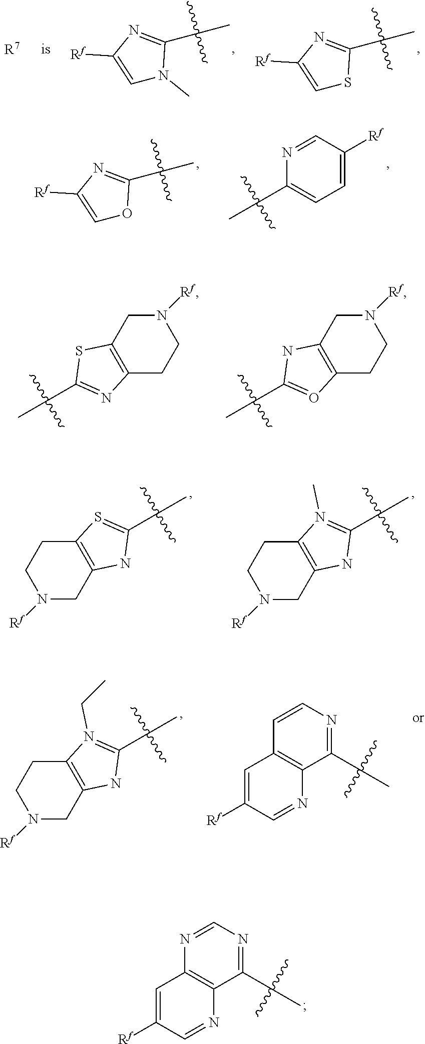
R^{a} and R^{b} are each independently —H, —CH₃ or —CH(CH₃)₂;
R^{d} is independently —CH(CH₃)₂, 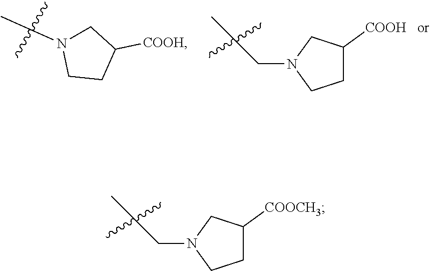 [SMILES: *N1CCC(C(=O)O)C1 ; *CN1CCC(C(=O)O)C1 ; *CN1CCC(C(=O)OC)C1]
R^{e} and R^{f} are each independently —H, —CH₃, —CH₂CH₃, —CH(CH₃)₂, —CH₂CH(CH₃)OH, —CH₂CH(OH)CH₃,

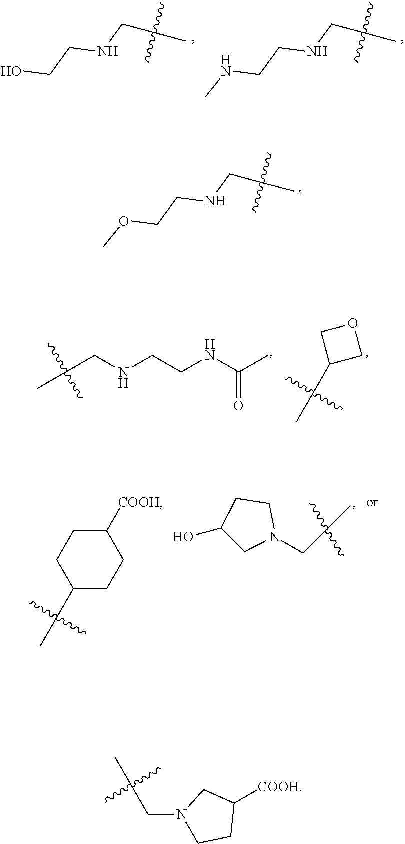

3. The compound according to claim 2, or a tautomer, mesomer, racemate, enantiomer, diastereomer, mixture of isomers, pharmaceutically acceptable salt, polymorph, solvate, prodrug, metabolite, or isotopic derivative thereof, wherein
R⁰ is —NH—C(O)—R⁶, —NH—R⁶, —N(CH₃)—C(O)—R⁶ or —N(CH(CH₃)₂)—C(O)—R⁶;
R⁵ is —NH—C(O)—R⁷, —N(CH₃)—C(O)—R⁷, —N(CH(CH₃)₂)—C(O)—R⁷,

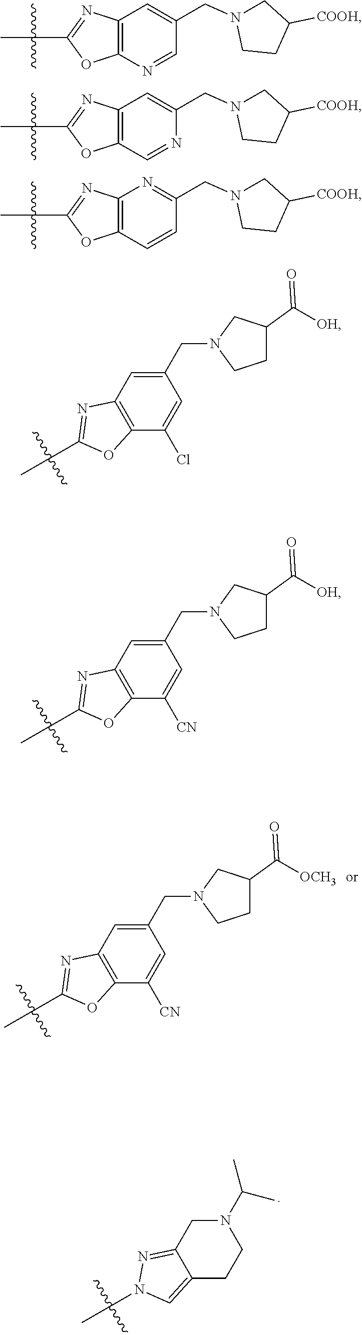

4. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
X¹ is C—R², X² is C, X³ is C;
R⁰ is —NH—C(O)—R⁶ or —NH—R⁶;
R¹ is —Cl or —CH₃;
R² is —H;
R³ is —Cl or —CH₃;
R⁴ is —H;
R⁵ is —NH—C(O)—R⁷, 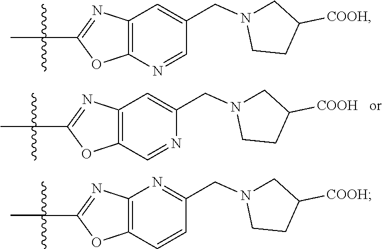
R⁶ is 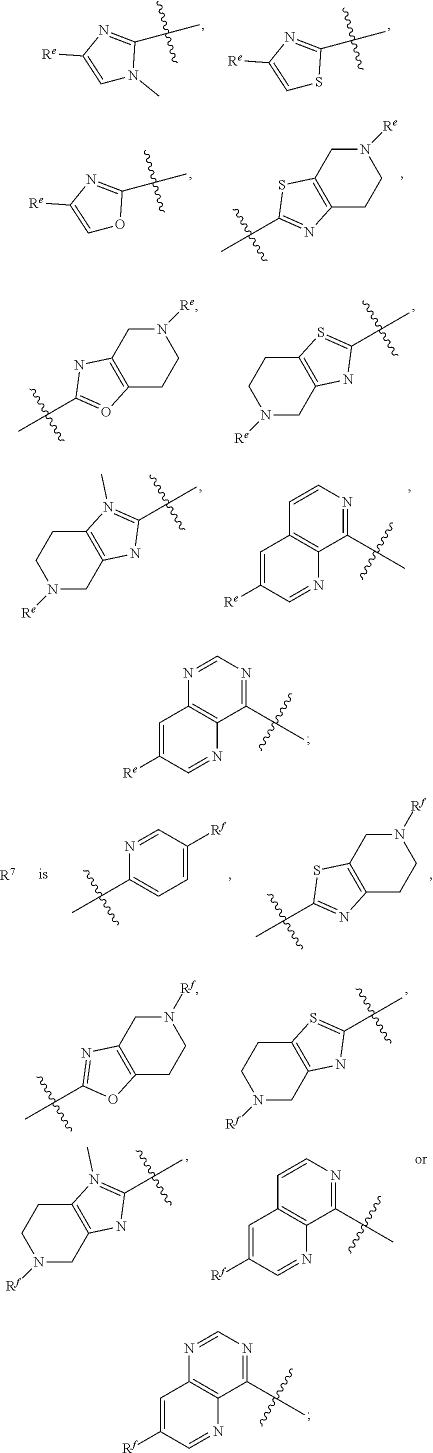
R^{e} is —H, —CH₃, —CH₂CH(CH₃)OH, 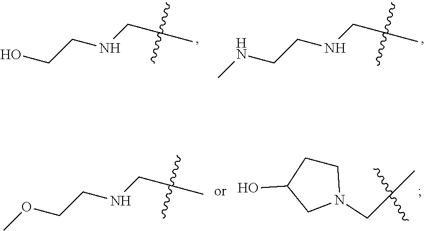
R^{f} is —CH₃,

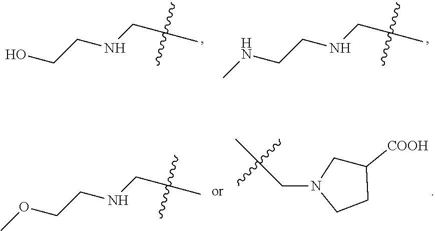

5. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
X¹ is N, X² is C, X³ is C;
R⁰ is —NH—C(O)—R⁶ or —NH—R⁶;
R¹ is —CH₃;
R³ is —Cl or —CH₃;
R⁴ is —H or —CH₃;
R⁵ is —NH—C(O)—R⁷, 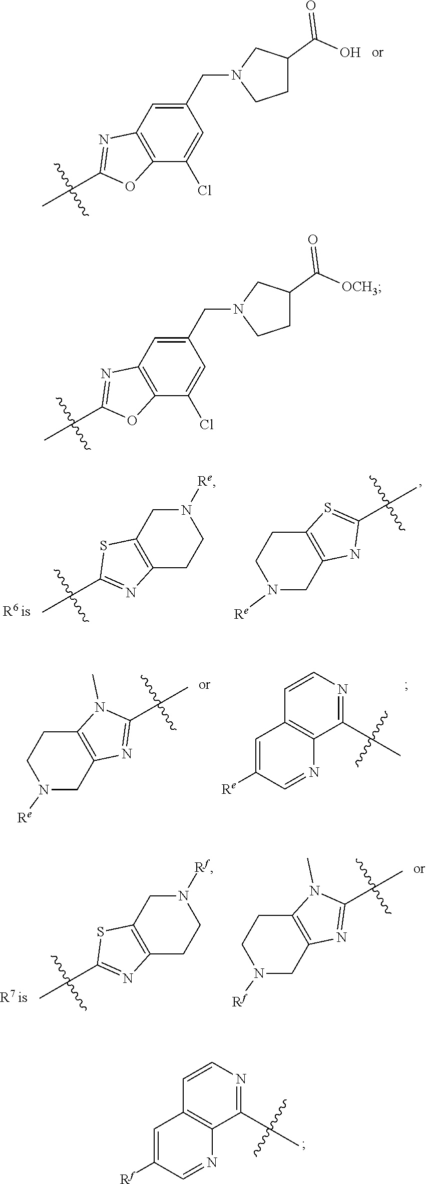
R^{e} is —H, —CH₃, —CH₂CH₃, —CH(CH₃)₂, —CH₂CH(CH₃)OH or 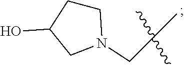 [SMILES: *CN1CCC(O)C1]
R^{f} is —CH₃, —CH₂CH₃, —CH(CH₃)₂, —CH₂CH(CH₃)OH, —CH₂CH(OH)CH₃,

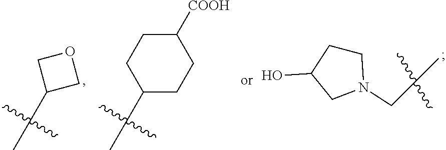 [SMILES: *C1COC1 ; *C1CCC(C(=O)O)CC1 ; *CN1CCC(O)C1]

6. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
X¹ is C—R², X² is C, X³ is N;
R⁰ is —NH—C(O)—R⁶;
R¹ is —Cl or —CH₃;
R² is —H;
R³ is —H, —Cl or —CH₃;
R⁴ is —H;
R⁵ is —NH—C(O)—R⁷;
R⁶ is 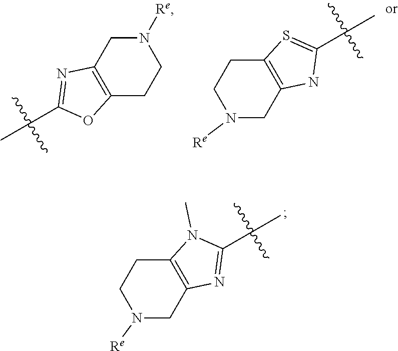
R⁷ is 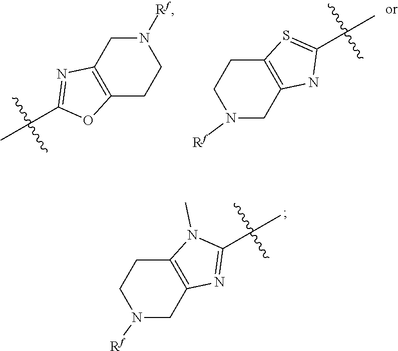
R^{e} is —CH₃ or —CH₂CH(CH₃)OH;
R^{f} is —CH₃ or —CH₂CH(CH₃)OH.

7. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
X¹ is C—R², X² is N, X³ is C;
R⁰ is —NH—C(O)—R⁶, —N(CH₃)—C(O)—R⁶ or —N(CH(CH₃)₂)—C(O)—R⁶;
R¹ is —Cl or —CH₃;
R² is —H, —F or —Cl;
R³ is —Cl or —CH₃;
R⁴ is —H;
R⁵ is —NH—C(O)—R⁷, —N(CH₃)—C(O)—R⁷ or —N(CH(CH₃)₂)—C(O)—R⁷;
R⁶ is 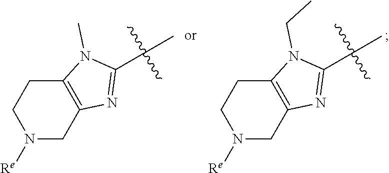
R⁷ is 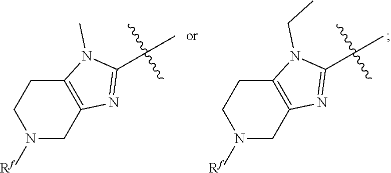
R^{e} is —CH₃ or —CH₂CH(CH₃)OH;
R^{f} is —CH₃.

8. The compound according to claim 1, or a tautomer, mesomer, racemate, enantiomer, diastereomer, mixture of isomers, pharmaceutically acceptable salt, polymorph, solvate, prodrug, metabolite, or isotopic derivative thereof, wherein
X¹ is C—R², X² is N, X³ is N;
R⁰ is —NH—C(O)—R⁶;
R¹ is —Cl or —CH₃;
R² is —H;
R³ is —Cl or —CH₃;
R⁴ is —H;
R⁵ is —NH—C(O)—R⁷;
R⁶ is 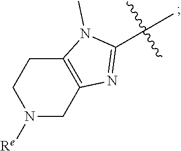
R⁷ is 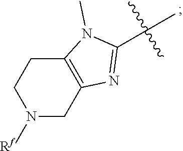
R^{e} is —CH₃ or —CH₂CH(CH₃)OH;
R^{f} is —CH₃ or —CH₂CH(CH₃)OH.

9. The compound according to claim 1, wherein said compound is selected from the group consisting of:
N-(2-chloro-3′-(4-(((2-hydroxyethyl)amino)methyl)-1-methyl-1H-imidazole-2-carboxamido)-2′-methyl-[1,1′-biphenyl]-3-yl)-5-(((2-hydroxyethyl)amino)methyl)picolinamide;
N-(2′-chloro-3′-(5-((((2-hydroxyethyl)amino)methyl)picolinamido)-2-methyl-[1,1′-biphenyl]-3-yl)-4-(((2-hydroxyethyl)amino)methyl)thiazole-2-carboxamide;
(S)—N-(5-(3-(1,5-dimethyl-4,5,6,7-tetrahydro-1H-imidazo[4,5-c]pyridine-2-carboxamido)-2-methylphenyl)-4-methylpyridin-3-yl)-5-(2-hydroxypropyl)-1-methyl-4,5,6,7-tetrahydro-1H-imidazo[4,5-c]pyridine-2-carboxamide;
(R)-1-(((2-(2,2′-dimethyl-3′-(5-methyl-4,5,6,7-tetrahydrothiazolo[5,4-c]pyridine-2-carboxamido))-[1,1′-biphenyl]-3-yl)oxazolo[5,4-b]pyridin-6-yl)methyl)pyrrolidine-3-carboxylic acid;
N-(5-(3-(1,5-dimethyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide;
N-(5-(3-(5-(2-hydroxypropyl)-1-methyl-4,5,6,7-tetrahydro-1H-imidazo[4,5-c]pyridine-2-carboxamido)-2-methylphenyl)-4-methylpyridin-3-yl)-5-methyl-4,5,6,7-tetrahydrothiazolo[5,4-c]pyridine-2-carboxamide;
(R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[5,4-b]pyridin-6-yl)methyl)pyrrolidine-3-carboxylic acid;
(R)-1-((7-chloro-2-(2-methyl-3-(4-methyl-5-(5-methyl-4,5,6,7-tetrahydrothiazolo[5,4-c]pyridine-2-carboxamido)pyridin-3-yl)phenyl)benzo[d]oxazol-5-yl)methyl)pyrrolidine-3-carboxylic acid;
(R)-1-(((7-chloro-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid;
(R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridin-6-yl)methyl)pyrrolidine-3-carboxylic acid;
(R)-1-(((7-chloro-2-(5-(3-((3-((((R)-3-hydroxypyrrolidin-1-yl)methyl)-1,7-naphthyridin-8-yl)amino)-2-methylphenyl)-4-methylpyridin-3-yl)benzo[d]oxazol-5-yl)methyl)pyrrolidine-3-carboxylic acid trifluoroacetate;
(R)-1-(((7-chloro-2-(5-(3-(5-((S)-2-hydroxypropyl))-1-methyl-4,5,6,7-tetrahydro-1H-imidazo[4,5-c]pyridine-2-carboxamide)-2-methylphenyl)-4-methylpyridin-3-yl)benzo[d]oxazol-5-yl)methyl)pyrrolidine-3-carboxylic acid;
(R)-1-(((7-chloro-2-(5-(3-(1,5-dimethyl-4,5,6,7-tetrahydro-1H-imidazo[4,5-c]pyridine-2-carboxamido)-2-methylphenyl)-4-methylpyridin-3-yl)benzo[d]oxazol-5-yl)methyl)pyrrolidine-3-carboxylic acid;
(R)-1-((2-(3′-(5-((S)-2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-b]pyridine-6-yl)methyl)pyrrolidine-3-carboxylic acid;
N-(3-chloro-2-(3-(5-(2-hydroxypropyl)-1-methyl-4,5,6,7-tetrahydro-1H-imidazo[4,5-c]pyridine-2-carboxamido))-2-methylphenyl)pyridin-4-yl)-1,5-dimethyl-4,5,6,7-tetrahydro-1H-imidazo[4,5-c]pyridine-2-carboxamide;
N-(3-(3-chloro-2-(1,5-dimethyl-4,5,6,7-tetrahydro-1H-imidazo[4,5-c]pyridine-2-carboxamido)pyridin-4-yl)-2-methylphenyl)-5-(2-hydroxypropyl)-1-methyl-4,5,6,7-tetrahydro-1H-imidazo[4,5-c]pyridine-2-carboxamide;
(R)-1-((2-(3′-((3-(((R)-3-hydroxypyrrolidin-1-yl)methyl)-1,7-diazanaphthalen-8-yl)amino)-2,2′-dimethyl-[1,1′-biphenyl]-3-yl)oxazolo[4,5-b]pyridin-5-yl)methyl)pyrrolidine-3-carboxylic acid trifluoroacetate;
(R)-1-((2-(2,2′-dimethyl-3′-(4,5,6,7-tetrahydrothiazolo[5,4-c]pyridine-2-carboxamido)-[1,1′-biphenyl]-3-yl)oxazolo[5,4-b]pyridin-6-yl)methyl)pyrrolidine-3-carboxylic acid trifluoroacetate;
(R)-1-(((7-chloro-2-(4-methyl-5-(2-methyl-3-(1-methyl-4,5,6,7-tetrahydro-1H-imidazole)[4,5-c]pyridine-2-carboxamido)phenyl)pyridin-3-yl)benzo[d]oxazol-5-yl)methyl)pyrrolidine-3-carboxylic acid;
methyl (R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylate;
(R)-1-(((7-chloro-2-(3-(5-(1,5-dimethyl-4,5,6,7-tetrahydro-1H-imidazo[4,5-c]pyridine-2-carboxamido)-4-methylpyridin-3-yl)-2-methylphenyl)benzo[d]oxazol-5-yl)methyl)pyrrolidine-3-carboxylic acid;
(R)-1-((2-(3′-(5-((S)-2-hydroxypropyl)-4,5,6,7-tetrahydrothiazolo[5,4-c]pyridine-2-carboxamido)-2,2′-dimethyl-[1,1′-biphenyl]-3-yl)oxazolo[5,4-b]pyridin-6-yl)methyl)pyrrolidine-3-carboxylic acid;
(S)—N-(5-(2-chloro-3-(1-methyl-5-(oxetan-3-yl)-4,5,6,7-tetrahydro-1H-imidazo[4,5-c]pyridine-2-carboxamido)phenyl)-4-methylpyridin-3-yl)-5-(2-hydroxypropyl)-1-methyl-4,5,6,7-tetrahydro-1H-imidazo[4,5-c]pyridine-2-carboxamide;
(S)—N-(2-chloro-3-(5-(5-(2-hydroxypropyl)-1-methyl-4,5,6,7-tetrahydro-1H-imidazo[4,5-c]pyridine-2-carboxamido)-4-methylpyridin-3-yl)phenyl)-5-isopropyl-1-methyl-4,5,6,7-tetrahydro-1H-imidazo[4,5-c]pyridine-2-carboxamide;
(R)-1-(((7-cyano-2-(4-methyl-5-(2-methyl-3-(5-methyl-4,5,6,7-tetrahydrothiazolo[5,4-c]pyridine-2-carboxamido)phenyl)pyridin-3-yl)benzo[d]oxazol-5-yl)methyl)pyrrolidine-3-carboxylic acid;
N-(5-(2-chloro-3-(5-((S)-2-hydroxypropyl)-1-methyl-4,5,6,7-tetrahydro-1H-imidazo[4,5-c]pyridine-2-carboxamido)phenyl)-4-methylpyridin-3-yl)-5-((S)-2-hydroxypropyl)-1-methyl-4,5,6,7-tetrahydro-1H-imidazo[4,5-c]pyridine-2-carboxamide;
N-(2-chloro-3-(5-(5-ethyl-1-methyl-4,5,6,7-tetrahydro-1H-imidazo[4,5-c]pyridine-2-carboxamido)-4-methylpyridin-3-yl)phenyl)-5-ethyl-1-methyl-4,5,6,7-tetrahydro-1H-imidazo[4,5-c]pyridine-2-carboxamide;
(R)-1-((2-(3′-(1,5-dimethyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid;
(R)-1-((2-(3′-(5-(2-hydroxypropyl)-1-methyl-4,5,6,7-tetrahydro-1H-imidazo[4,5-c]pyridine-2-carboxamido)-2,2′-dimethyl-[1,1′-biphenyl]-3-yl)oxazolo[5,4-c]pyridin-6-yl)methyl)pyrrolidine-3-carboxylic acid; and
(R)-1-(((7-chloro-2-(3-(5-(5-((S)-2-hydroxypropyl))-1-methyl-4,5,6,7-tetrahydro-1H-imidazo[4,5-c]pyridine-2-carboxamido)-4-methylpyridin-3-yl)-2-methylphenyl)benzo[d]oxazole-5-yl)methyl)pyrrolidine-3-carboxylic acid.

10. A method for preparing the compound represented by general formula I according to claim 1, wherein the compound represented by general formula I is prepared by the following scheme: 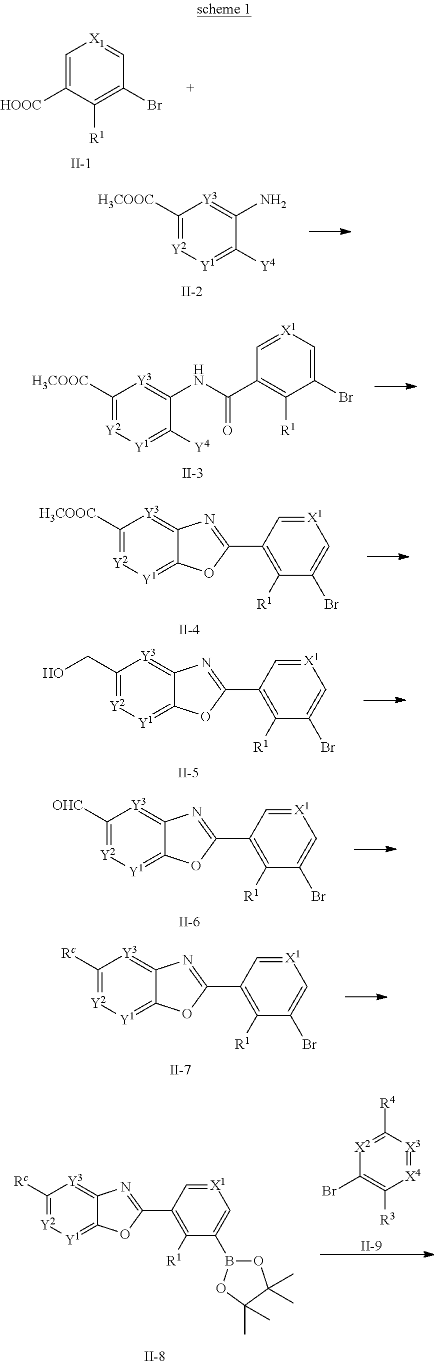 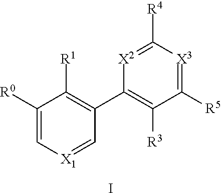
wherein Y₁, Y₂ and Y₃ are N or C, in which at least two of Y₁, Y₂ and Y₃ are C, or wherein Y₁ is CR, in which R is halogen or —CN;
Y₄ is fluoro, chloro, bromo or iodo; and said scheme 1 comprises:
reacting compound (II-1) with a corresponding arylamine (II-2) in the presence of a condensing agent under an alkaline condition to obtain compound (II-3); performing a ring-closing reaction on compound (II-3) under the catalysis of a copper salt to obtain compound (II-4); subjecting compound (II-4) to a three-step reaction including reduction, oxidation and reductive amination to obtain compound (II-7); reacting compound (II-7) with diboron pinacol ester in the presence of a catalyst under heating and alkaline conditions to obtain compound (II-8); reacting compound (II-8) with compound (II-9) in the presence of a catalyst under heating and alkaline conditions to obtain compound (I); wherein,
alternatively, compound (II-4) is synthesized by: 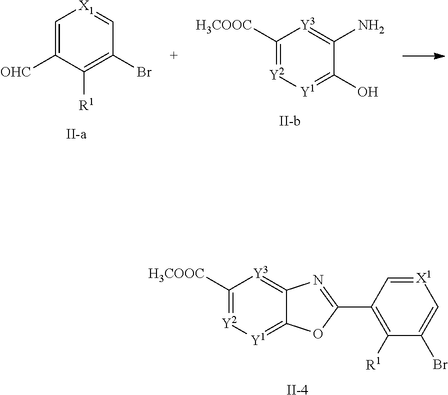
performing a ring-closing reaction by directly heating compounds (II-a) and (II-b) to obtain compound (II-4); 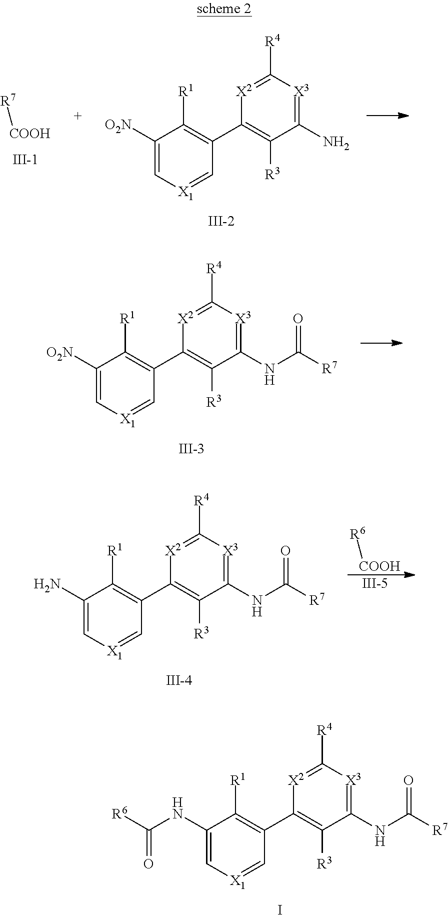
said scheme 2 comprises: reacting compound (III-1) with a corresponding arylamine (III-2) in the presence of a condensing agent under an alkaline condition to obtain compound (III-3); reducing compound (III-3) by a reducing agent, and then reacting it with a corresponding acid (III-5) under an alkaline condition in the presence of a condensing agent to obtain compound (I).

11. (canceled)

12. A method for inhibiting PD-1 and/or PD-L1, comprising administrating the compound represented by general formula (I) according to claim 1, or a tautomer, mesomer, racemate, enantiomer, diastereomer, mixture of isomers, pharmaceutically acceptable salt, polymorph, solvate, prodrug, metabolite, or isotopic derivative thereof to a subject in need thereof.

13. A pharmaceutical composition, comprising a therapeutically and/or prophylactically effective amount of the compound represented by general formula (I) according to claim 1, or a tautomer, mesomer, racemate, enantiomer, diastereomer, mixture of isomers, pharmaceutically acceptable salt, polymorph, solvate, prodrug, metabolite, or isotopic derivative thereof, and pharmaceutically acceptable carriers and/or diluents.

14. A method for preventing, alleviating, or treating cancer, infection or autoimmune disease, comprising administering a compound represented by general formula (I) according to claim 1, or a tautomer, mesomer, racemate, enantiomer, diastereomer, mixture of isomers, pharmaceutically acceptable salt, polymorph, solvate, prodrug, metabolite, or isotopic derivative thereof, to a subject in need thereof.

15. (canceled)

16. (canceled)

17. The method according to claim 14, wherein the cancer is one or more of brain tumor, nasopharyngeal cancer, lung cancer, breast cancer, cervical cancer, esophageal cancer, stomach cancer, liver cancer, colorectal cancer, blood cancer and bone cancer.
